(19) United States
(12) Patent Application Publication
(10) Pub. No.: US 2007/0032531 A1
(43) Pub. Date: Feb. 8, 2007

(54) SPHINGOSINE KINASE INHIBITORS AND METHODS OF THEIR USE

(75) Inventors: Charles D. Smith, Charleston, SC (US); Kevin J. French, Harrisburg, PA (US); Zuping Xia, Hershey, PA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Apogee Biotechnology Corporation, Hershey, PA (US)

(21) Appl. No.: 11/462,153

(22) Filed: Aug. 3, 2006

Related U.S. Application Data
(60) Provisional application No. 60/705,608, filed on Aug. 4, 2005.

(51) Int. Cl. A61K 31/4439; A61K 31/427; A61K 31/426; C07D 417/02

(52) U.S. Cl. 514/342; 514/365; 548/200; 546/269.7

(57) ABSTRACT

The invention relates to compounds, pharmaceutical compositions thereof, and methods for inhibiting sphingosine kinase and for treating or preventing hyperproliferative disease, inflammatory disease, or angiogenic disease,

### REFERENCE TO RELATED APPLICATIONS

This application is a non-provisional application claiming priority under 35 U.S.C. §119(e) to provisional application No. 60/705,608 filed Aug. 4, 2005, the contents of which are incorporated herein by reference

### GOVERNMENT SPONSORSHIP

This invention was made with government support Grant R43 CA097833 awarded by the United States Public Health Service, Accordingly, the US government may have certain rights in this invention,

### FIELD OF THE INVENTION

The invention relates to compounds that are capable of inhibiting sphingosine kinase and to processes for the synthesis of these compounds. The invention also relates to pharmaceutical compositions comprising these compounds and to methods for the use of these compounds and pharmaceutical composition for treating or preventing hyperproliferative disease, inflammatory disease, or angiogenic disease.

### BACKGROUND OF THE INVENTION

The mechanisms and effects of the interconversion of sphingolipids have been the subjects of a growing body of scientific investigation Sphingomyelin is not only a building block for cellular membranes but also serves as the precursor for potent lipid messengers that have profound cellular effects. As described below, stimulus-induced metabolism of these lipids is critically involved in the biology of hyperproliferative, inflammatory and angiogenic diseases. Consequently, manipulation of these metabolic pathways is a novel method for the therapy of a variety of diseases

Ceramide is produced by the hydrolysis of sphingomyelin in response to several stimuli, including growth factors and inflammatory cytokines. Ceramide induces apoptosis in cancerous cells. Additionally, ceramide can be hydrolyzed by the action of ceramidase to produce sphingosine. Sphingosine is then phosphorylated by sphingosine kinase (SK) to produce sphingosine-1-phosphate (S1P). Evidence demonstrates that S1P is a critical second messenger that exerts proliferative and anti-apoptotic actions Additionally, ceramide enhances apoptosis in response to anticancer drugs including Taxol and etoposide Furthermore, ceramide appears to induce apoptosis in tumor cells without killing quiescent normal cells. Studies in various cell lines consistently indicate that S1P is able to induce proliferation and protect cells from apoptosis. Together, the data demonstrate that the balance between cellular levels of ceramide and S1P determines whether a cancer cell proliferates or dies by apoptosis. Therefore, altering this balance by reducing the production of S1P within hyperproliferating cells is an effective method to treat disorders arising from abnormal cell proliferation.

Sphingosine kinase is responsible for S1P production in cells. RNA encoding SK is expressed in most tissues, with higher levels often occurring in tumor tissue than in corresponding normal tissue. A variety of proliferative factors, including Protein Kinase C (PKC) activators, fetal calf serum, Platelet-Derived Growth Factor, Epidermal Growth Factor, and Tumor Necrosis Factor-alpha (TNFα) rapidly elevate cellular SK activity This promotes proliferation and inhibits apoptosis of the target cells. Additionally, an oncogenic role of SK has been demonstrated, In these studies, transfection of SK into NIH/3T3 fibroblasts was sufficient to promote foci formation and cell growth in soft-agar, and to allow these cells to form tumors in NOD/SCID mice. Additionally, inhibition of SK by transfection with a dominant-negative SK mutant or by treatment of cells with the nonspecific SK inhibitor D-erythro-N,N-dimethylsphingosine (DMS) blocked transformation mediated by oncogenic H-Ras. Since abnormal activation of Ras, as well as overexpression and mutation of ras family genes, frequently occurs in cancer, these findings indicate a significant role of SK in this disease,

In addition to its role in regulating cell proliferation and apoptosis, S1P has been shown to have several important effects on cells that mediate immune functions. Platelets, monocytes and mast cells secrete S1P upon activation, promoting inflammatory cascades at the site of tissue damage. Activation of SK is required for the signaling responses since the ability of TNFα to induce adhesion molecule expression via activation of Nuclear Factor Kappa (NFκB) is mimicked by S1P and is blocked by DMS. Similarly, S1P mimics the ability of TNFα to induce the expression of Cyclooxygenase-2 (COX-2) and the synthesis of prostaglandin E2 (PGE₂), and knock-down of SK by RNA interference blocks these responses to TNFα but not S1P. S1P is also a mediator of Ca²⁺ influx during neutrophil activation by TNFα and other stimuli, leading to the production of superoxide and other toxic radicals. Therefore, reducing the production of S1P within immune cells and their target tissues may be an effective method to treat disorders arising from abnormal inflammation. Examples of such disorders include inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity,

Angiogenesis refers to the state in the body in which various growth factors or other stimuli promote the formation of new blood vessels, and this process is critical to the pathology of a variety of diseases. In each case, excessive angiogenesis allows the progression of the disease and/or the produces undesired effects in the patient. Since conserved biochemical mechanisms regulate the proliferation of vascular endothelial cells that form these new blood vessels, identification of methods to inhibit these mechanisms are expected to have utility for the treatment and prevention of a variety of diseases More specifically, certain growth factors have been identified that lead to the pathogenic angiogenesis. For example, Vascular Endothelial Growth Factor (VEGF) has angiogenic and mitogenic capabilities. Specifically, VEGF induces vascular endothelial cell proliferation, favoring the formation of new blood vessels. Sphingosine kinase is an important mediator of the actions of VEGF. For example, SK has been shown to mediate VEGF-induced activation of protein kinases. VEGF has also been shown to specifically induce S1P receptors, associated with enhanced intracellular signaling responses to S1P and the potentiation of its angiogenic actions. Production of S1P by SK stimulates NFκB activity leading to the production of COX-2, adhesion molecules and additional VEGF production, all of which promote angiogenesis. Furthermore, the expression of endothelial isoforms of nitric oxide synthase (eNOS) is regulated by SK, and eNOS too subsequently modulates angiogenesis. Therefore, reducing the production of S1P within endothelial cells is likely to be an effective method to treat disorders arising from abnormal angiogenesis. Examples of such disorders include arthritis, cancer, psoriasis, Kaposi's sarcoma, hemangiomas, myocardial angiogenesis, atherosclerosis, and ocular angiogenic diseases.

In spite of the high level of interest in sphingolipid-derived signaling, there are very few known inhibitors of the enzymes of this pathway and the utility of pharmacologic inhibition of SK ii vivo has not been previously demonstrated. In particular, the field suffers from a lack of potent and selective inhibitors of SK. Pharmacological studies to date have used three compounds to inhibit SK activity: DMS, D,L-threo-dihydrosphingosine and N,N,N-trimethyl-sphingosine. However, these compounds are not specific inhibitors of SK and have been shown to inhibit several other protein and lipid kinases. Therefore, improved inhibitors of SK are required for use as antiproliferative, anti-inflammatory and anti-angiogenic agents. In this application, we describe novel compounds that display these desirable activities.

### SUMMARY OF THE INVENTION

The invention encompasses the compounds of formula I, formula II, formula III and formula IV, shown below, processes for the synthesis of these compounds, pharmaceutical compositions containing such compounds and methods employing such compounds or compositions in the treatment or prevention of hyperproliferative disease, inflammatory disease, or angiogenic disease, and more specifically compounds that are capable of inhibiting SK.

In one aspect, the invention provides compounds of formula I: 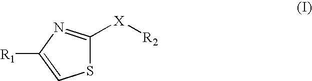
and pharmaceutically acceptable salts thereof, wherein

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

R₁ and R₂ are independently H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —NH₃, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₃R₄, —OC(O)NR₃R₄, —NR₃C(O)R₄, —CF₃, —OCF₃, —OH, C₁-C₆ alkoxy, hydroxyalkyl, —CN, —CO₂H, —SH, —S-alkyl, —SOR₃R₄, —SO₂R₃R₄, —NO₂, or NR₃R₄; and

R₃ is H, alkyl, preferably lower alkyl, or oxo, provided that when R₃ and R₄ are on the same carbon, and R₃ is oxo, then R₄ is absent;

R₄ and R₅ are independently H or alkyl, preferably lower alkyl

Another aspect of the invention provides compounds of formula II: 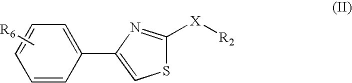
and pharmaceutically acceptable salts thereof, wherein:

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₃R₄, —OC(O)NR₃R₄, —NR₃C(O)R₄, —CF₃, —OCF₃, —OH, C₁-C₆ alkoxy, hydroxyalkyl, —CN, —CO₂H, —SH, —S-alkyl, —SOR₃R₄, —SO₂R₃R₄, —NO₂, or NR₃R₄;

R₃ is H, alkyl, preferably lower alkyl, or oxo, provided that when R₃ and R₄ are on the same carbon, and R₃ is oxo, then R₄ is absent;

R₄ and R₅ are independently H or alkyl, preferably lower alkyl; and

R₆ is halogen, haloalkyl, alkoxy, haloalkoxy, hydroxyalkyl, alkanoyl, —COOH, —OH, —SH, —S-alkyl, —CN, —NO₂, or —NH₂.

Another aspect of the invention provides compounds of formula III: 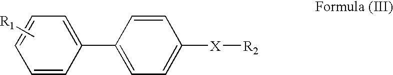
and pharmaceutically acceptable salts thereof, wherein:

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

R₁ is halogen, haloalkyl, alkoxy, haloalkoxy, hydroxyalkyl, alkanoyl, —COOH, —OH, —SH, —S-alkyl, —CN, —NO₂, or —NH₂.

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SN, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OC₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl.

Another aspect of the invention provides compounds of formula IV: 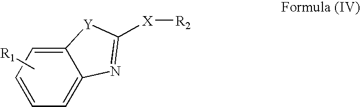
and pharmaceutically acceptable salts thereof, wherein:

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

Y is O or S;

R₁ is halogen, haloalkyl, alkoxy, haloalkoxy, hydroxyalkyl, alkanoyl, —COON, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COON, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SN, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl

The invention also provides pharmaceutical compositions comprising a compound or salt of formula I, II, III or IV and at least one pharmaceutically acceptable carrier, solvent, adjuvant or diluent.

The invention also provides methods for the treatment or prevention of hyperproliferative disease, inflammatory disease, or angiogenic disease.

The invention also provides methods for inhibiting sphingosine kinase in a cell

The compounds of the invention are potent and selective inhibitors of SK, Therefore, the invention provides inhibitors of SK which are useful as antiproliferative, anti-inflammatory and anti-angiogenic agents,

Specific preferred embodiments of the invention will become evident from the following more detailed description of certain preferred embodiments and claims.

### DESCRIPTION OF THE DRAWINGS

FIG. 1 Inhibition of tumor growth by SK inhibitors. Balb/c female mice were injected subcutaneously with JC murine adenocarcinoma cells suspended in PBS. After palpable tumor growth, animals were treated by intraperitoneal injection of either 0.1 mL of 50% DMSO (control, circles) or 50 mg/kg of Compound 8 (squares) or Compound 73 (triangles) on odd numbered days. Whole body weight and tumor volume measurement were performed for up to 18 days., * p<0.05. Inset: Averaged body weights of mice from each group during course of study.

FIG. 2. Dose-response relationships for inhibition of tumor growth by Compound 8, Balb/c female mice were injected subcutaneously with JC cells suspended in PBS. After palpable tumor growth, animals were treated by oral gavage of either 100 μl of PEG400 (control, circles) or Compound 8 at 3.5 mg/kg (diamonds), 10 mg/kg (inverted triangles), 35 mg/kg (triangles) or 100 mg/kg (squares) on odd numbered days. Whole body weight and tumor volume measurement were performed for up to 18 days.

FIG. 3, Inhibition of TNFα-induced Cox-2 activity by Compound 8. Rat IEC6 cells (Panel A) or human endothelial cells (Panel B) were incubated for 18 hours with dimethylsulfoxide (DMSO) as a solvent control, or 100 ng of TNFα/mL in the presence of DMSO or 10 μg/mL of Compound 8. Levels of PGE₂ secreted into the medium were quantified by ELISA Values represent the mean±sd for triplicate samples in a typical experiment.

FIG. 4. Effects of Compound 8 and Dipentum on the DAI in the acute DSS-colitis model. C57BL/6 mice were treated for 6 days as follows: normal drinking water and daily oral administration of PEG (No DSS), 2% DSS in the drinking water and daily oral administration of PEG (DSS alone); 2% DSS in the drinking water and daily oral administration of 50 mg/kg Compound 8 in PEG (DSS+Compound 8), or 2% DSS in the drinking water and daily oral administration of 50 mg/kg Dipentum in PEG (DSS+Dipentum). On the indicated day, the Disease Activity Index was calculated for each group. Values represent the mean±sd for 5-6 mice per group.

FIG. 5. Effects of Compound 8 and Dipentum on colon length in the acute DSS-colitis model. Mice from the experiment described in FIG. 4 were sacrificed on Day 6, and the colon was harvested from each animal and measured. Data represent the mean±sd colon length.

FIG. 6. Effects of Compound 8 and Dipentum on neutrophil infiltration into the colon in the acute DSS-colitis model. Myeloperoxidase (MPO) activity from the colons of the animals described in FIG. 4 was measured. Values the mean±sd MPO activity in units per gram of tissue.

FIG. 7. Effects of Compound 8 and Dipentum on colonic cytokine levels in the acute DSS-colitis model. Colon samples from mice described in FIG. 4 were extracted and assayed for the levels of the indicated cytokines. Values represent the mean±sd amount of each cytokine in 4-5 samples per group,

FIG. 8. Effects of Compound 8 on the drug-activity index (DAI) in the chronic DSS-colitis model. Mice received 2 cycles (7 days per cycle) of DSS (1.5% cycle 1 and 1% cycle 2), 2 cycles of normal drinking water and were randomized by DAI on Day 28 into groups of 8 mice. The mice were then treated as follows: No DSS (▪)—normal drinking water and orally dosed with PEG400 every day for 7 days (water control); DSS alone (▴)—drinking water containing 1.5% DSS and orally dosed with PEG daily for 7 days; DSS +Coumpound 8 (▾)—drinking water containing 1.5% DSS and orally dosed with Coumpound 8 (50 mg/kg) every day for 7 days; DSS+Dipentum (♦)—drinking water containing 1.5% DSS and orally dosed with Dipentum (50 mg/kg). *p<0.001 versus No DSS group.

FIG. 9. Effects of Compound 8 on S1P levels in the colons of the animals in the chronic DSS-colitis model. Colon samples from mice described in FIG. 8 were extracted and assayed for the levels of S1P by LC/MS/MS. Values represent the mean±sd for 8 samples per group; * p<0.05 versus No DSS group.

FIG. 10. Effects of Compound 8 on disease progression in the collagen-induced arthritis (CIA) model in mice. Female DBA/1 mice were injected with collagen, boosted after 3 weeks and then monitored for symptoms of arthritis. Upon disease manifestation, groups of mice were treated for 12 days as follows: (▴) Compound 8 (50 mg/kg given orally each day for 6 days per week); or (▪) vehicle (PEG400 given under the same schedule). On the indicated Day of treatment, the average clinical score (A) and the average hind paw diameter (B) was determined. * p≦0.05 versus PEG400 alone group,

### DESCRIPTION OF THE PREFERRED EMBODIMENT

All patents and publications referred to herein are hereby incorporated by reference for all purposes,

Unless the substituents for a particular formula are expressly defined for that formula, they are understood to carry the definitions set forth in connection with the preceding formula to which the particular formula makes reference.

As noted above, the invention provides compounds of formula I: 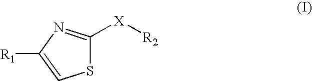
and pharmaceutically acceptable salts thereof, wherein

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOHN —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above R₁ and R₂ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-allyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂; and

R₃ is H, alkyl, preferably lower alkyl, or oxo, provided that when R₃ and R₄ are on the same carbon, and R₃ is oxo, then R₄ is absent;

R₄ and R₅ are independently H or alkyl, preferably lower alkyl.

Preferred compounds of the formula I include those described by formula II: 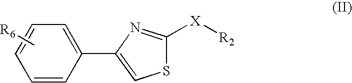
and pharmaceutically acceptable salts thereof, wherein:

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the allyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅;

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl; and

R₆ is halogen, haloalkyl, alkoxy, haloalkoxy, hydroxyalkyl, alkanoyl, —COOH, —OH, —SH, —S-alkyl, —CN, —NO₂, or —NH₂.

More preferred compounds of the formula II include those wherein:

X is —C(R₃,R₄)N(R₅)—, —C(O)N(R₄)—, —N(R₄)C(O)—, or —C(R₄,R₅)—;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl and

R₆ is halogen, haloalkyl, alkoxy, haloalkoxy, hydroxyalkyl, alkanoyl, —COOH, —OH, —SH, —S-alkyl, —CN, —NO₂, or —NH₂.

Most preferred compounds of the formula II include those wherein:

X is —C(O)N(R₄)— or —N(R₄)C(O)—;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (αO), —COOH, —OH, —SH, —S-alkyl, —CN, —NO₂, —NO₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₄ and R₅ are independently H or (C₁-C₆)alkyl and

R₆ is halogen, haloalkyl, alkoxy, haloalkoxy, hydroxyalkyl, alkanoyl, —COOH, —OH, —SH, —S-alkyl, —CN, —NO₂, or —NH₂. The invention also provides compounds of formula III: 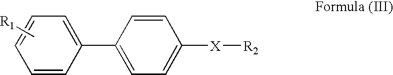
and pharmaceutically acceptable salts thereof, wherein:

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, S(O)₂N(R₄)— or —N(R₄)S(O)₂—;

R₁ is halogen, haloalkyl, alkoxy, haloalkoxy, hydroxyalkyl, alkanoyl, —COOH, —OH, —SH, —S-alkyl, —CN, —NO₂, or NH₂:

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COON, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ allyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is N, alkyl, preferably lower alkyl, or oxo, provided that when R₃ and R₄ are on the same carbon, and R₃ is oxo, then R₄ is absent;

R₄ and R₅ are independently H or (C₁-C₆)alkyl.

Preferred compounds of the formula III include those wherein:

X is —C(R₃,R₄)N(R₅)—, —C(O)N(R₄)—, —N(R₄)C(O)—, or —C(R₄,R₅)—;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, allyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH,₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl.

Most preferred compounds of the formula III include those wherein:

X is —C(O)N(R₄)— or —N(R₄)C(O)—;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl.

The invention also provides compounds of formula IV: 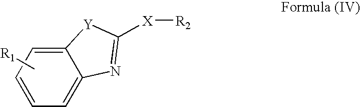
and pharmaceutically acceptable salts thereof, wherein:

X is —C(R₃,R₄)N(R₅)—, —C(O)N(R₄)—, —N(R)C(O)—, —C(R₄,R₅)—, —N(R₄)—, —O—, —S—, —C(O)—, —S(O)₂—, —S(O)₂N(R₄)— or —N(R₄)S(O)₂—;

Y is O or S;

R₁ is halogen, haloalkyl, alkoxy, haloalkoxy, hydroxyalkyl, alkanoyl, —COOH, —OH, —SH, —S-allyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl

Preferred compounds of the formula IV include those wherein:

X is —C(R₃,R₄)N(R₅)—, —C(O)N(R₄)—, —N(R₄)C(O)—, or —C(R₄,R₅)—;

Y is O or S;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl.

Most preferred compounds of the formula IV include those wherein:

X is —C(O)N(R₄)— or —N(R₄)C(O)—;

Y is O or S;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkylheterocycloalkyl, acyl, aroyl, halogen, haloalkyl, alkoxy, haloalkoxy, hydroxyalkyl, alkanoyl, oxo (═O), —COOOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)R₅, —NR₄C(O)R₅, —CF₃, —OCF₃, OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃, R₄ and R₅ are independently H or (C₁-C₆)alkyl.

The invention also provides methods for treating a patient who has, or in preventing a patient from getting, a disease or condition including but not limited to a hyperproliferative disease, an inflammatory disease, or an angiogenic disease, which methods include administration of a therapeutically effective amount of a compound of formula (I), (II), (III), or (IV) or a pharmaceutically acceptable salt thereof, to a patient in need of such treatment or prevention.

One preferred hyperproliferative disease which the compounds of the invention are useful in treating or preventing is cancer, including as non-limiting examples thereof solid tumors such as head and neck cancers, lung cancers, gastrointestinal tract cancers, breast cancers, gynecologic cancers, testicular cancers, urinary tract cancers, neurological cancers, endocrine cancers, skin cancers, sarcomas, mediastinal cancers, retroperitoneal cancers, cardiovascular cancers, mastocytosis, carcinosarcomas, cylindroma, dental cancers, esthesioneuroblastoma, urachal cancer, Merkel cell carcinoma and paragangliomas, and hematopoietic cancers such as Hodgkin lymphoma, non-Hodgkin lymphoma, chronic leukemias, acute leukemias, myeloproliferative cancers, plasma cell dyscrasias, and myelodysplastic syndromes. The foregoing list is by way of example, and is not intended to be exhaustive or limiting.

Other preferred diseases which can be treated or prevented with the compounds of the invention include inflammatory diseases, such as inter alia inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity, including allergic encephalomyelitis, allergic neuritis, transplant allograft rejection, graft versus host disease, myocarditis, thyroiditis, nephritis, systemic lupus erythematosus, and insulin-dependent diabetes mellitus.

Other preferred diseases which can be treated or prevented with the compounds of the invention include angiogenic diseases, such as diabetic retinopathy, arthritis, psoriasis, Kaposi's sarcoma, hemangiomas, myocardial angiogenesis, atherosclerotic plaque neovascularization, and ocular angiogenic diseases such as choroidal neovascularization, retinopathy of prematurity (retrolental fibroplasias), macular degeneration, corneal graft rejection, rubeosis, neuroscular glacoma and Oster Webber syndrome.

The invention also provides pharmaceutical compositions that include a compound of formula (I), (II), (III) or (IV) or a pharmaceutically acceptable salt thereof as active ingredients, in combination with a pharmaceutically acceptable carrier, medium, or auxiliary agent.

The pharmaceutical compositions of the present invention may be prepared in various forms for administration, including tablets, caplets, pills or dragees, or can be filled in suitable containers, such as capsules, or, in the case of suspensions, filled into bottles. As used herein “pharmaceutically acceptable carrier medium” includes any and all solvents, diluents, or other liquid vehicle; dispersion or suspension aids; surface active agents; preservatives; solid binders; lubricants and the like, as suited to the particular dosage form desired. Various vehicles and carriers used in formulating pharmaceutical compositions and known techniques for the preparation thereof are disclosed in Remington's Pharmaceutical Sciences (Osol et al. eds., 15th ed., Mack Publishing Co,: Easton, Pa., 1975). Except insofar as any conventional carrier medium is incompatible with the chemical compounds of the present invention, such as by producing any undesirable biological effect or otherwise interacting in a deleterious manner with any other component of the pharmaceutical composition, the use of the carrier medium is contemplated to be within the scope of this invention,

In the pharmaceutical compositions of the present invention, the active agent may be present in an amount of at least 1% and not more than 99% by weight, based on the total weight of the composition, including carrier medium or auxiliary agents, Preferably, the proportion of active agent varies between 1 % to 70% by weight of the composition.

Pharmaceutical organic or inorganic solid or liquid carrier media suitable for enteral or parenteral administration can be used to make up the composition. Gelatin, lactose, starch, magnesium, stearate, talc, vegetable and animal fats and oils, gum polyalkylene glycol, or other known excipients or diluents for medicaments may all be suitable as carrier media.

The pharmaceutical compositions of the present invention may be administered using any amount and any route of administration effective for treating a patient who has, or in preventing a patient from getting, a disease or condition selected from the group consisting of a hyperproliferative disease, an inflammatory disease, and an angiogenic disease Thus the expression “therapeutically effective amount” as used herein, refers to a sufficient amount of the active agent to provide the desired effect against target cells. The exact amount required will vary from subject to subject, depending on the species, age, and general condition of the subject; the particular SK inhibitor; its mode of administration; and the like.

The pharmaceutical compounds of the present invention are preferably formulated in dosage unit form for ease of administration and uniformity of dosage. “Dosage unit form,” as used herein, refers to a physically discrete unit of therapeutic agent appropriate for the animal to be treated. Each dosage should contain the quantity of active material calculated to produce the desired therapeutic effect either as such, or in association with the selected pharmaceutical carrier medium. Typically, the pharmaceutical composition will be administered in dosage units containing from about 0.1 mg to about 10,000 mg of the agent with a range of about 1 mg to about 1000 mg being preferred.

The pharmaceutical compositions of the present invention may be administered orally or paternally, such as by intramuscular injection, intraperitoneal injection, or intravenous infusion. The pharmaceutical compositions may be administered orally or parenterally at dosage levels of about 0.1 to about 1000 mg/kg, and preferably from about 1 to about 100 mg/kg, of animal body weight per day, one or more times a day, to obtain the desired therapeutic effect.

Although the pharmaceutical compositions of the present invention can be administered to any subject that can benefit from the therapeutic effects of the compositions, the compositions are intended particularly for the treatment of diseases in humans.

The pharmaceutical compositions of the present invention will typically be administered from 1 to 4 times a day, so as to deliver the daily dosage as described herein. Alternatively, dosages within these ranges can be administered by constant infusion over an extended period of time, usually 1 to 96 hours, until the desired therapeutic benefits have been obtained. However, the exact regimen for administration of the chemical compounds and pharmaceutical compositions described herein will necessarily be dependent on the needs of the animal being treated, the type of treatments being administered, and the judgment of the attending physician.

In certain situations, the compounds of this invention may contain one or more asymmetric carbon atoms, so that the compounds can exist in different stereoisomeric forms. These compounds can be, for example, racemates, chiral non-racemic or diastereomers. In these situations, the single enantiomers, i e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

Non-toxic pharmaceutically acceptable salts of the compounds of the present invention include, but are not limited to salts of inorganic acids such as hydrochloric, sulfuric, phosphoric, diphosphoric, hydrobromic, and nitric or salts of organic acids such as formic, citric, malic, maleic, fumaric, tartaric, succinic, acetic, lactic, methanesulfonic, p-toluenesulfonic, 2-hydroxyethylsulfonic, salicylic and stearic. Similarly, pharmaceutically acceptable cations include, but are not limited to sodium, potassium, calcium, aluminum, lithium and ammonium Those skilled in the art will recognize a wide variety of non-toxic pharmaceutically acceptable addition salts. The invention also encompasses prodrugs of the compounds of the present invention.

The invention also encompasses prodrugs of the compounds of the present invention Those skilled in the art will recognize various synthetic methodologies, which may be employed to prepare non-toxic pharmaceutically acceptable addition salts and prodrugs of the compounds encompassed by the present invention.

The invention provides compounds of formula I, II, III and IV which are inhibitors of SK, and which are useful for modulating the sphingomyelin signal transduction pathway, and in treating and preventing hyperproliferative diseases, inflammatory diseases, and angiogenic diseases. The compounds of the invention can be prepared by one skilled in the art based only on knowledge of the compound's chemical structure. The chemistry for the preparation of the compounds of this invention is known to those skilled in the art In fact, there is more than one process to prepare the compounds of the invention. Specific examples of methods of preparation can be found herein and in the art.

As discussed above, sphingolipids are critically important in regulating the balance between cell proliferation and apoptosis. Sphingosine 1-phosphate is produced by the enzyme SK and stimulates the proliferation of tumor cells. Concurrent depletion of ceramide by the action of SK blocks apoptosis. The compounds of the invention are inhibitors of human SK. Therefore, inhibition of SK activity according to the invention will attenuate tumor cell proliferation and promote apoptosis. Therefore, the compounds of the invention are useful as anticancer agents. Furthermore, since cell hyperproliferation is a required process in the development of atherosclerosis and psoriasis, the compounds of the invention, which are SK inhibitors, are useful in the treatment of these, and other, hyperproliferative diseases Additionally, inappropriate activation and/or proliferation of specific classes of lymphocytes results in chronic inflammatory and autoimmune diseases. Consequently, compounds of the invention are also useful in the treatment of these diseases. Additionally, inappropriate angiogenesis results in a variety of diseases, as described below. Consequently, compounds of the invention are also useful in the treatment of these diseases

Definitions

The definitions and explanations below are for the terms as used throughout this entire document, including both the specification and the claims.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise The symbol “—” in general represents a bond between two atoms in the chain Thus CH₃—O—CH₂—CH(Rᵢ)—CH₃ represents a 2-substituted-1-methoxypropane compound. In addition, the symbol “—” represents the point of attachment of the substituent to a compound. Thus for example aryl(C₁-C₆)alkyl-indicates an alkylaryl group, such as benzyl, attached to the compound at the alkyl moiety.

Where multiple substituents are indicated as being attached to a structure, it is to be understood that the substituents can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

The phrase “optionally substituted” is used interchangeably with the phrase “substituted or unsubstituted”. Unless otherwise indicated, an optionally substituted group may have a substituent at each substitutable position of the group, and each substituent is independent of the other.

As used herein, the terms “halogen” or “halo” indicate fluorine, chlorine, bromine, or iodine,

The term “heteroatom” means nitrogen, oxygen or sulfur and includes any oxidized form of nitrogen and sulfur, and the quaternized form of any basic nitrogen. Also the term “nitrogen” includes a substitutable nitrogen in a heterocyclic ring. As an example, in a saturated or partially unsaturated ring having 0-3 heteroatoms selected from nitrogen, oxygen or sulfur, the nitrogen may be N (as in 3,4-dihydro-2H-pyrrolyl), NH (as in pyrrolidinyl) or NR⁺ (as in N-substituted pyrrolidinyl).

The term “alkyl”, as used herein alone or as part of a larger moiety, refers to a saturated aliphatic hydrocarbon including straight chain, branched chain or cyclic (also called “cycloalkyl”) groups Examples of alkyl groups include methyl, ethyl, propyl, isopropyl, butyl, iso-, sec- and tert-butyl, pentyl, hexyl, heptyl, 3-ethylbutyl, and the like. Preferably, the alkyl group has 1 to 20 carbon atoms (whenever a numerical range, e.g. “1-20”, is stated herein, it means that the group, in this case the alkyl group, may contain 1 carbon atom, 2 carbon atoms, 3 carbon atoms, etc up to and including 20 carbon atoms). More preferably, it is a medium size alkyl having 1 to 10 carbon atoms. Most preferably, it is a lower alkyl having 1 to 4 carbon atoms. The cycloalkyl can be monocyclic, or a polycyclic fused system. Examples of cycloalkyl groups include cyclopropyl, cyclobutyl, cyclopentyl, cyclohexyl, cycloheptyl, cyclooctyl, and adamantyl. The alkyl or cycloalkyl group may be unsubstituted or substituted with 1, 2, 3 or more substituents. Examples of such substituents including, without limitation, halo, hydroxy, amino, alkoxy, alkylamino, dialkylamino, cycloalkyl, aryl, aryloxy, arylalkyloxy, heterocyclic radical, and (heterocyclic radical)oxy Examples include fluoromethyl, hydroxyethyl, 2,3-dihydroxyethyl, (2- or 3-furanyl)methyl, cyclopropylmethyl, benzyloxyethyl, (3-pyridinyl)methyl, (2-thienyl)ethyl, hydroxypropyl, aminocyclohexyl, 2-dimethylaminobutyl, methoxymethyl, N-pyridinylethyl, and diethylaminoethyl.

The term “cycloalkylalkyl”, as used herein alone or as part of a larger moiety, refers to a C₃-C₁₀ cycloalkyl group attached to the parent molecular moiety through an alkyl group, as defined above Examples of cycloalkylalkyl groups include cyclopropylmethyl and cyclopentylethyl.

The term “alkenyl”, as used herein alone or as part of a larger moiety, refers to an aliphatic hydrocarbon having at least one carbon-carbon double bond, including straight chain, branched chain or cyclic groups having at least one carbon-carbon double bond. Preferably, the alkenyl group has 2 to 20 carbon atoms. More preferably, it is a medium size alkenyl having 2 to 10 carbon atoms. Most preferably, it is a lower alkenyl having 2 to 6 carbon atoms. The alkenyl group may be unsubstituted or substituted with 1, 2, 3 or more substituents. Examples of such substituents including, without limitation halo, hydroxy, amino, alkoxy, alkylamino, dialkylamino, cycloalkly, aryl, aryloxy, arylalkyloxy, heterocyclic radical, and (heterocyclic radical)oxy. Depending on the placement of the double bond and substituents, if any, the geometry of the double bond may be entgegen (E) or zusammen (Z), cis, or trans. Examples of alkenyl groups include ethenyl, propenyl, cis-2-butenyl, trans-2-butenyl, and 2-hyroxy-2-propenyl,

The term “alkynyl”, as used herein alone or as part of a larger moiety, refers to an aliphatic hydrocarbon having at least one carbon-carbon triple bond, including straight chain, branched chain or cyclic groups having at least one carbon-carbon triple bond. Preferably, the alkynyl group has 2 to 20 carbon atoms. More preferably, it is a medium size alkynyl having 2 to 10 carbon atoms. Most preferably, it is a lower alkynyl having 2 to 6 carbon atoms. The alkynyl group may be unsubstituted or substituted with 1, 2, 3 or more substituents. Examples of such substituents including, without limitation, halo, hydroxy, amino, alkoxy, alkylamino, dialkylamino, cycloalkly, aryl, aryloxy, arylalkyloxy, heterocyclic radical, and (heterocyclic radical)oxy. Examples of alkynyl groups include ethynyl, propynyl, 2-butynyl, and 2-hyroxy3-butylnyl.

The term “alkoxy”, as used herein alone or as part of a larger moiety, represents an alkyl group of indicated number of carbon atoms attached to the parent molecular moiety through an oxygen bridge Examples of alkoxy groups include, for example, methoxy, ethoxy, propoxy and isopropoxy. Alkoxy radicals may be further substituted with one or more halo atoms, such as fluoro, chloro or bromo, to provide “haloalkoxy” radicals. Examples of such radicals include fluoromethoxy, chloromethoxy, trifluoromethoxy, and fluoroethoxy.

The term “aryl”, as used herein alone or as part of a larger moiety, refers to an aromatic hydrocarbon ring system containing at least one aromatic ring. The aromatic ring may optionally be fused or otherwise attached to other aromatic hydrocarbon rings or non-aromatic hydrocarbon rings. Additionally, the aryl group may be substituted or unsubstituted by various groups such as hydrogen, halo, hydroxy, alkyl, haloalkyl, alkoxy, nitro, cyano, alkyl amine, carboxy or alkoxycarbonyl. Examples of aryl groups include, for example, phenyl, naphthyl, 1,2,3,4-tetrahydronaphthalene, benzodioxole, and biphenyl. Preferred examples of unsubstituted aryl groups include phenyl and biphenyl. Preferred aryl group substituents include hydrogen, halo, alkyl, haloalkyl, hydroxy and alkoxy.

The term “heteroalkyl”, as used herein alone or as part of a larger moiety, refers to an alkyl radical as defined herein with one or more heteroatoms replacing a carbon atom with the moiety. Such heteroalkyl groups are alternately referred to using the terms ether, thioether, amine, and the like.

The term “heterocyclyl”, as used herein alone or as part of a larger moiety, refers to saturated, partially unsaturated and unsaturated heteroatom-containing ring shaped radicals, where the heteroatoms may be selected from nitrogen, sulfur and oxygen. Said heterocyclyl groups may be unsubstituted or substituted at one or more atoms within the ring system. The heterocyclic ring may contain one or more oxo groups.

The term “heterocycloalkyl”, as used herein alone or as part of a larger moiety, refers to a non-aromatic ring system containing at least one heteroatom selected from nitrogen, oxygen, and sulfur. The heterocycloalkyl ring may be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred monocyclic heterocycloalkyl groups include piperidyl, piperazinyl, morpholinyl, pyrrolidinyl, thiomorpholinyl, thiazolidinyl, 1,3-dioxolanyl, 1,4-dioxanyl, tetrahydrofuranyl, tetrahydrothiophenyl, tetrahydrothiopyranyl, and the likes. Heterocycloalkyl radicals may also be partially unsaturated. Examples of such groups include dihydrothienyl, dihydropyranyl, dihydrofuryl, and dihydrothiazolyl.

The term “heteroaryl”, as used herein alone or as part of a larger moiety, refers to an aromatic ring system containing at least one heteroatom selected from nitrogen, oxygen, and sulfur. The heteroaryl ring may be fused or otherwise attached to one or More heteroaryl rings, aromatic or non-aromatic hydrocarbon rings or heterocycloalkyl rings. Additionally, the heteroaryl group may be unsubstituted or substituted at one or more atoms of the ring system, or may contain one or more oxo groups. Examples of heteroaryl groups include, for example, pyridine, furan, thiophene, carbazole and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, benzopyrazolyl, purinyl, benzooxazolyl, and carbazolyl.

The term “acyl” means an H—C(O)— or alkyl-C(O)— group in which the alkyl group, straight chain, branched or cyclic, is as previously described. Exemplary acyl groups include formyl, acetyl, propanoyl, 2-methylpropanoyl, butanoyl, and caproyl.

The term “aroyl” means an aryl-C(O)— group in which the aryl group is as previously described. Exemplary aroyl groups include benzoyl and 1- and 2-naphthloyl The term “solvate” means a physical association of a compound of this invention with one or more solvent molecules. This physical association involves varying degress of ionic and covalent bonding, including hydrogen bonding In certain instances, the solvate will be capable of isolation, for example when one or more solvent molecules are incorporated in the crystal lattice of the crystalline solid. “Solvate” encompasses both solution-phase and isolatable solvates. Exemplary solvates include ethanolates, methanolates, and the like. “Hydrate” is a solvate wherein the solvent molecule(s) is/are H₂O.

Compounds that have the same molecular formula but differ in the nature or sequence of bonding of their atoms or arrangement of their atoms in space are termed “isomers”, Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, a carbon atom that is bonded to four different groups, a pair of enantiomers is possible An enantiomer can be characterized by the absolute configuration of its asymmetric center and is described by the R— and S-sequencing rules of Cahn and Prelog, which are well known to those in the art. Additionally, entiomers can be characterized by the manner in which a solution of the compound rotates a plane of polarized light and designated as dextrorotatory or levorotatory (i.e. as (+) or (−) isomers respectively). A chiral compound can exist as either individual enantiomer or as a mixture thereof A mixture containing equal proportions of the enantiomers is called a “racemic mixture”.

The compounds of this invention may possess one or more asymmetric centers; such compounds can therefore be produced as individual (R)— or (S)-stereoisomers or as mixtures thereof. Unless otherwise indicated, the specification and claims is intended to include both individual enantiomers as well as mixtures, racemic or otherwise, thereof.

Certain compounds of this invention may exhibit the phenomena of tautomerism and/or structural isomerism. For example, certain compounds described herein may adopt an E or a Z configuration about a carbon-carbon double bond or they may be a mixture of E and Z. This invention encompasses any tautomeric or structural isomeric form and mixtures thereof.

Unless otherwise stated, structures depicted herein are also meant to include compounds that differ only in the presence of one or more isotopically enriched atoms For example, compounds having the present structures except for the replacement of a hydrogen by a deuterium or tritium, or the replacement of a carbon by a ¹³C— or ¹⁴C-enriched carbon are within the scope of this invention Such compounds are useful, for example, as analytical tools or probes in biologic assays.

As used herein, “SK-related disorder”, “SK-driven disorder”, and “abnormal SK activity” all refer to a condition characterized by inappropriate, i.e, under or, more commonly, over, SK catalytic activity. Inappropriate catalytic activity can arise as the result of either: (1) SK expression in cells that normally do not express SK, (2) increased SK catalytic activity leading to unwanted cellular process, such as, without limitation, cell proliferation, gene regulation, resistance to apoptosis, and/or differentiation. Such changes in SK expression may occur by increased expression of SK and/or mutation of SK such that its catalytic activity is enhanced, (3) decreased SK catalytic activity leading to unwanted reductions in cellular processes. Some examples of SK-related disorders, without limitation, are described elsewhere in this application.

The term “method” refers to manners, means, techniques and procedures for accomplishing a given task including, but not limited to, those manners, means, techniques and procedures either known to, or readily developed from known manners, means, techniques and procedures by practitioners of the chemical, pharmaceutical, biological, biochemical and medical arts.

The term “modulation” or “modulating” refers to the alteration of the catalytic activity of SK. In particular, modulating refers to the activation or, preferably, inhibition of SK catalytic activity, depending on the concentration of the compound or salt to which SK is exposed.

The term “catalytic activity” as used herein refers to the rate of phosphorylation of sphingosine under the influence of SK.

The term “contacting” as used herein refers to bringing a compound of this invention and SK together in such a manner that the compound can affect the catalytic activity of SK, either directly, ire., by interacting with SK itself, or indirectly, i.e., by altering the intracellular localization of SK. Such “contacting” can be accomplished in vitro, i.e. in a test tube, a Petri dish or the like. In a test tube, contacting may involve only a compound and SK or it may involve whole cells. Cells may also be maintained or grown in cell culture dishes and contacted with a compound in that environment. In this context, the ability of a particular compound to affect an SK-related disorder can be determined before the use of the compounds in vivo with more complex living organisms is attempted For cells outside the organism, multiple methods exist, and are well-known to those skilled in the art, to allow contact of the compounds with SK including, but not limited to, direct cell microinjection and numerous techniques for promoting the movement of compounds across a biological membrane.

The term “in vitro” as used herein refers to procedures performed in an artificial environment, such as for example, without limitation, in a test tube or cell culture system. The skilled artisan will understand that, for example, an isolate SK enzyme may be contacted with a modulator in an in vitro environment. Alternatively, an isolated cell may be contacted with a modulator in an in vitro environment.

The term “in vivo” as used herein refers to procedures performed within a living organism such as, without limitation, a human, mouse, rat, rabbit, bovine, equine, porcine, canine, feline, or primate.

The term “IC₅₀” or “50% inhibitory concentration” as used herein refers to the concentration of a compound that reduces a biological process by 50%. These processes can include, but are not limited to, enzymatic reactions, i.e. inhibition of SK catalytic activity, or cellular properties, i.e. cell proliferation, apoptosis or cellular production of S1P.

As used herein, “administer” or “administration” refers to the delivery of a compound or salt of the present invention or of a pharmaceutical composition containing a compound or salt of this invention to an organism for the purpose of prevention or treatment of an SK-related disorder.

As used herein, the terms “prevent,” “preventing” and “prevention” refer to a method for barring an organism from acquiring an SK-related disorder.

As used herein, the terms “treat,” “treating” and “treatment” refer to a method of alleviating or abrogating an SK-mediated disorder and/or its attendant symptoms.

The term “organism” refers to any living entity comprised of at least one cell. A living organism can be as simple as, for example, a single eukaryotic cell or as complex as a mammal. In a preferred aspect of this invention, the organism is a mammal. In a particularly preferred aspect of this invention, the mammal is a human being.

A “Pharmaceutical composition” refers to a mixture of one or more of the compounds described herein, or pharmaceutically acceptable salts thereof, with other chemical components, such as physiologically acceptable carriers and excipients. The purpose of a pharmaceutical composition is to facilitate administration of a compound to an organism.

The term “pharmaceutically acceptable salt” refers to those salts that retain the biological effectiveness of the parent compound. Such salts include: (1) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid, or malonic acid and the like, preferably hydrochloric acid or (L)-malic acid; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, tromethamine, N-methylglucamine, and the like.

As used herein, the term a “physiologically acceptable carrier” refers to a carrier or diluent that does not cause significant irritation to an organism and does not abrogate the biological activity and properties of the administered compound. Typically, this includes those properties and/or substances that are acceptable to the patient from a pharmacological/toxicological point of view and to the manufacturing pharmaceutical chemist from a physical/chemical point of view regarding composition, formulation, stability, patient acceptance and bioavailability.

An “excipient” refers to an inert substance added to a pharmaceutical composition to further facilitate administration of a compound Example, without limitations, of excipients include calcium carbonate, calcium phosphate, various sugars and types of'starch, cellulose derivatives (including microcrystalline cellulose), gelatin, vegetable oils, polyethylene glycols, diluents, granulating agents, lubricants, binders, disintegrating agents, and the like.

The term “therapeutically effective amount” as used herein refers to that amount of the compound being administered that is effective to reduce or lessen at least one symptom of the disease being treated or to reduce or delay onset of one or more clinical markers or symptoms of the disease. In reference to the treatment of cancer, a therapeutically effective amount refers to that amount that has the effect of. (1) reducing the size of the tumor, (2) inhibiting, i.e. slowing to some extent, preferably stopping, tumor metastasis, (3) inhibiting, i.e. slowing to some extent, preferably stopping, tumor growth, and/or (4) relieving to some extent, preferably eliminating, one or more symptoms associated with the cancer.

The compounds of this invention may also act as a prodrug The term “prodrug” refers to an agent which is converted into the parent drug in vivo. Prodrugs are often useful because, in some situations, they may be easier to administer than the parent drug. They may, for example, be bioavailable by oral administration whereas the parent drug is not The prodrug may also have improved solubility in pharmaceutical compositions over the parent drug. An example, without limitation, of a prodrug would be a compound of the present invention which is administered as an ester (the “prodrug”), carbamate or urea.

The compounds of this invention may also be metabolized by enzymes in the body of the organism, such as a human being, to generate a metabolite that can modulate the activity of SK. Such metabolites are within the scope of the present invention,

Indications

Sphingosine kinase (SK), whose catalytic activity is modulated by the compounds and compositions of this invention, is a key enzyme involved in signaling pathways that are abnormally activated in a variety of diseases The following discussion outlines the roles of SK in hyperproliferative, inflammatory and angiogenic diseases, and consequently provides examples of uses of the compounds and compositions of this invention The use of these compounds and compositions for the prevention and/or treatment of additional diseases in which SK is abnormally activated are also within the scope of the present invention

Hyperproliferative Diseases.

The present invention relates to compounds, pharmaceutical compositions and methods useful for the treatment and/or prevention of hyperproliferative diseases. More specifically, the invention relates to compounds and pharmaceutical compositions that inhibit the enzymatic activity of SK for the treatment and/or prevention of hyperproliferative diseases, such as cancer, psoriasis, mesangial cell proliferative disorders, atherosclerosis and restenosis. The following discussion demonstrates the role of SK in several of these hyperproliferative diseases. Since the same processes are involved in the above listed diseases, the compounds, pharmaceutical compositions and methods of this invention will be useful for the treatment and/or prevention of a variety of diseases.

Sphingosine-1-phosphate and ceramide have opposing effects on cancer cell proliferation and apoptosis. Sphingomyelin is not only a building block for cellular membranes but also serves as the precursor for potent lipid messengers that have profound cellular effects Stimulus-induced metabolism of these lipids is critically involved in cancer cell biology. Consequently, these metabolic pathways offer exciting targets for the development of anticancer drugs.

Ceramide is produced by the hydrolysis of sphingomyelin in response to growth factors or other stimuli Ceramide induces apoptosis in tumor cells, but can be further hydrolyzed by the action of ceramidase to produce sphingosine. Sphingosine is then rapidly phosphorylated by SK to produce S1P, which is a critical second messenger that exerts proliferative and antiapoptotic actions. For example, microinjection of S1P into mouse oocytes induces DNA synthesis. Additionally, S1P effectively inhibits ceramide-induced apoptosis in association with decreased caspase activation Furthermore, ceramide enhances apoptosis in response to anticancer drugs including Taxol and etoposide. These studies in various cell lines consistently indicate that S1P is able to induce proliferation and protect cells from ceramide-induced apoptosis.

A critical balance, which may be termed a ceramide/S1P rheostat, has been hypothesized to determine the fate of the cell. In this model, the balance between the cellular concentrations of ceramide and S1P determines whether a cell proliferates or undergoes apoptosis. Upon exposure to mitogens or intracellular oncoproteins, the cells experience a rapid increase in the intracellular levels of S1P and depletion of ceramide levels. This situation promotes cell survival and proliferation. In contrast, activation of sphingomyelinase in the absence of activation of ceramidase and/or SK results in the accumulation of ceramide and subsequent apoptosis.

SK is the enzyme responsible for S1P production in cells. RNA encoding SK is detected in most tissues. A variety of proliferative factors, including PKC activators, fetal calf serum and platelet-derived growth factor (Olivera et al. 1993, Nature 365: 557), EGF and TNFα (Dressler et al., 1992, Science 255: 1715) rapidly elevate cellular SK activity SK activity is increased by phosphorylation of the enzyme by ERK (Pitson et al., 2003, Embo J 22: 5491), while S1P promotes signaling through the Ras-Raf-Mek-Erk pathway, setting up an amplification cascade for cell proliferation.

Sphingosine kinase and S1P play important roles in cancer pathogenesis. An oncogenic role of SK has been demonstrated. In these studies, transfection of SK into NIH/3T3 fibroblasts was sufficient to promote foci formation and cell growth in soft-agar, and to allow these cells to form tumors in NOD/SCID mice (Xia et ale, 2000, Curr Biol 10: 1527). Additionally, inhibition of SK by transfection with a dominant-negative SK mutant or by treatment of cells with the nonspecific SK inhibitor DMS blocked transformation mediated by oncogenic H-Ras. As abnormal activation of Ras frequently occurs in cancer, these findings suggest a significant role of SK in this disease. SK has also been linked to estrogen signaling and estrogen-dependent tumorigenesis in MCF-7 cells (Nava et ah, 2002, Exp Cell Res 281: 115). Other pathways or targets to which SK activity has been linked in hyperproliferative diseases include VEGF signaling via the Ras and MAP kinase pathway (Shu et al., 2002, Mol Cell Biol 22: 7758), protein kinase C (Nakade et al., 2003, Biochim Biophys Acta 1635: 104), TNFα (Vann et al., 2002, J Biol Chem 277: 12649), hepatocyte nuclear factor-1 and retinoic acid receptor alpha, intracellular calcium and caspase activation. While the elucidation of downstream targets of S1P remains an interesting problem in cell biology, sufficient validation of these pathways has been established to justify the development of SK inhibitors as new types of antiproliferative drugs.

Cellular hyperproliferation is a characteristic of a variety of diseases, including, without limitation, cancer, psoriasis, mesangial cell proliferative disorders, atherosclerosis and restenosis. Therefore, the compounds, pharmaceutical compositions and methods of this invention will be useful for the prevention and/or treatment of cancer, including solid tumors, hematopoietic cancers and tumor metastases. Such cancers may include, without limitation, solid tumors such as head and neck cancers, lung cancers, gastrointestinal tract cancers, breast cancers, gynecologic cancers, testicular cancers, urinary tract cancers, neurological cancers, endocrine cancers, skin cancers, sarcomas, mediastinal cancers, retroperitoneal cancers, cardiovascular cancers, mastocytosis, carcinosarcomas, cylindroma, dental cancers, esthesioneuroblastoma, urachal cancer, Merkel cell carcinoma and paragangliomas. Additionally, such cancers may include, without limitation, hematopoietic cancers such as Hodgkin lymphoma, non-Hodgkin lymphoma, chronic leukemias, acute leukemias, myeloproliferative cancers, plasma cell dyscrasias, and myelodysplastic syndromes.

Psoriasis is a common chronic disfiguring skin disease that is characterized by well-demarcated, red, hardened and scaly plaques that may be limited or widespread. While the disease is rarely fatal, it has serious detrimental effects on the quality of life of the patient, and this is further complicated by the lack of effective therapies. There is therefore a large unmet need for effective and safe drugs for this condition. Psoriasis is characterized by local keratinocyte hyperproliferation. T cell-mediated inflammation and by localized angiogenesis. Abnormal activation of SK has been implicated in all of these processes. Therefore, SK inhibitors are expected to be of use in the therapy of psoriasis.

Mesangial cell hyperproliferative disorders refer to disorders brought about by the abnormal hyperproliferation of mesangial cells in the kidney. Mesangial hyperproliferative disorders include various human renal diseases such as glomerulneophritis, diabetic nephropathy, and malignant nephrosclerosis, as well as such disorders such as thrombotic microangiopathy syndromes, transplant rejection, and glomerulopathies. As the hyperproliferation of mesangial cells is induced by growth factors whose action is dependent on increased signaling through SK, the SK inhibitory compounds, pharmaceutical compositions and methods of this invention are expected to be of use in the therapy of these mesangial cell hyperproliferative disorders.

In addition to inflammatory processes discussed below, atherosclerosis and restenosis are characterized by hyperproliferation of vascular smooth muscle cells at the sites of the lesions. As the hyperproliferation of vascular smooth muscle cells is induced by growth factors whose action is dependent of increased signaling through SK, the SK inhibitory compounds, pharmaceutical compositions and methods of this invention are expected to be of use in the therapy of these vascular disorders

Inflammatory Diseases.

The present invention also relates to compounds, pharmaceutical compositions and methods useful for the treatment and/or prevention of inflammatory diseases More specifically, the invention relates to compounds and pharmaceutical compositions that inhibit the enzymatic activity of SK for the treatment and/or prevention of inflammatory diseases, such as inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity, including allergic encephalomyelitis, allergic neuritis, transplant allograft rejection, graft versus host disease, myocarditis, thyroiditis, nephritis, systemic lupus erythematosus, and insulin-dependent diabetes mellitus. The following discussion demonstrates the role of SK in several of these inflammatory diseases Since the same processes are involved in the above listed diseases, the compounds, pharmaceutical compositions and methods of this invention will be useful for the treatment and/or prevention of a variety of diseases.

Inflammatory bowel disease (IBD) encompasses a group of disorders characterized by pathological inflammation of the lower intestine. Crohn's disease and ulcerative colitis are the best-known forms of IBD, and both fall into the category of “idiopathic” IBD because their etiologies remain to be elucidated, although proposed mechanisms implicate infectious and immunologic mediators. Studies on the etiology and therapy of IBD have been greatly facilitated by the development of several animal models that mimic the clinical and immunopathological disorders seen in humans. From studies with these models, it is clear that the full manifestations of IBD are dependent on synergy between the humoral and cellular immune responses The notion that immune cells and cytokines play critical roles in the pathogenesis of IBD is well established; however, the molecular mechanisms by which this occurs are not yet clearly defined. As discussed below, cytokines that promote inflammation in the intestine afflicted with IBD), all activate a common mediator, sphingosine kinase (SK). Most prominently, tumor necrosis factor-α (TNFα) has been shown to play a significant role in IBD, such that antibody therapy directed against this cytokine, i.e. Remicade, may be a promising treatment. TNFα activates several processes shown to contribute to IBD and is necessary for both the initiation and persistence of the Th1 response. For example, TNFα has been shown act through the induction of nuclear factor kappa B NFκB) which has been implicated in increasing the proinflammatory enzymes nitric oxide synthase (NOS) and cyclooxygenase-2 (COX-2). COX-2 has been shown to play a key role in the inflammation of IBDs through its production of prostaglandins, and oxidative stress such as that mediated by nitric oxide produced by NOS has also shown to exacerbate IBD inflammation.

A common pathway of immune activation in IBDs is the local influx of mast cells, monocytes, macrophages and polymorphonuclear neutrophils which results in the secondary amplification of the inflammation process and produces the clinical manifestations of the diseases. This results in markedly increased numbers of mast cells in the mucosa of the ileum and colon of patients with IBD, which is accompanied by dramatic increases in TNFα (He, 2004, World J Gastroenterology 10 (3): 309). Additional mast cell secretory products, including histamine and tryptase, may be important in IBDs. Therefore, it is clear that inflammatory cascades play critical roles in the pathology of IBDs.

The mechanisms and effects of the sphingolipid interconversion have been the subjects of a growing body of scientific investigation. Sphingomyelin is not only a structural component of cellular membranes, but also serves as the precursor for the potent bioactive lipids ceramide and sphingosine I-phosphate (S1P). A ceramide : S1P rheostat is thought to determine the fate of the cell, such that the relative cellular concentrations of ceramide and S1P determine whether a cell proliferates or undergoes apoptosis. Ceramide is produced by the hydrolysis of sphingomyelin in response to inflammatory stresses, including TNFα, and can be hydrolyzed by ceramidase to produce sphingosine. Sphingosine is then rapidly phosphorylated by sphingosine kinase (SK) to produce S1P. Ceramidase and SK are also activated by cytokines and growth factors, leading to rapid increases in the intracellular levels of S1P and depletion of ceramide levels. This situation promotes cell proliferation and inhibits apoptosis. Deregulation of apoptosis in phagocytes is an important component of the chronic inflammatory state in IBDs, and S1P has been shown to protect neutrophils from apoptosis in response to Fas, TNFα and ceramide. Similarly, apoptosis of macrophages is blocked by S1P.

In addition to its role in regulating cell proliferation and apoptosis, S1P has been shown to have several important effects on cells that mediate immune functions Platelets, monocytes and mast cells secrete S1P upon activation, promoting inflammatory cascades at the site of tissue damage Activation of SK is required for the signaling responses, since the ability of TNFα to induce adhesion molecule expression via activation of NFκB is mimicked by S1P and is blocked by the SK inhibitor dimethylsphinigosine (Xia et ale, 1998, Proc Natl Acad Sci USA 95: 14196). Similarly, S1P mimics the ability of TNFα to induce the expression of COX-2 and the synthesis of PGE₂, and knock-down of SK by RNA interference blocks these responses to TNFα but not S1P (Pettus et al., 2003, FASEB J 17: 1411). S1P is also a mediator of Ca²⁺ influx during neutrophil activation by TNFα and other stimuli, leading to the production of superoxide and other toxic radicals (Mackinnon, 2002, Journal of Immunology 169(11): 6394).

A model for the roles of sphingolipid metabolites in the pathology of IBDs involves a combination of events in the colon epithelial cells and recruited mast cells, macrophages and neutrophils. Early in the disease, immunologic reactions or other activating signals promote the release of inflammatory cytokines, particularly TNFα from macrophages and mast cells The actions of TNFα are mediated through its activation of S1P production For example, TNFα induces S1P production in endothelial cells (Xia et al., 1998, Proc Natl Acad Sci USA 95: 14196), neutrophils (Niwa et al., 2000, Life Sci 66: 245) and monocytes by activation of sphingomyelinase, ceramidase and SK. S1P is a central player in the pathway since it has pleiotropic actions on the mucosal epithelial cells, macrophages, mast cells and neutrophils. Within the mucosal cells, S1P activates NFκB thereby inducing the expression of adhesion molecules, COX-2 resulting in PGE₂ synthesis, and NOS producing nitric oxide Together, these chemoattractants and the adhesion molecules promote neutrophil infiltration into the mucosa. At the same time, S1P activates the neutrophils resulting in the release of oxygen free radicals that further inflame and destroy epithelial tissue. Similarly, S1P promotes the activation and degranulation of mast cells.

As the processes involved in IBDs are induced by cytokines and growth factors whose action is dependent on increased signaling through SK, the SK inhibitory compounds, pharmaceutical compositions and methods of this invention are expected to be of use in the therapy of IBDs.

Rheumatoid arthritis (RA) is a chronic, systemic disease that is characterized by synovial hyperplasia, massive cellular infiltration, erosion of the cartilage and bone, and an abnormal immune response. Studies on the etiology and therapy of rheumatoid arthritis have been greatly facilitated by the development of animal models that mimic the clinical and immunopathological disorders seen in humans. From studies in these models, it is clear that the full manifestations of RA are dependent on synergy between the humoral and cellular immune responses. The notion that immune cells, especially neutrophils, and cytokines play critical roles in the pathogenesis of arthritis is well established. However, the mechanisms by which this occurs are not fully elucidated.

The early phase of rheumatic inflammation is characterized by leukocyte infiltration into tissues, especially by neutrophils. In the case of RA, this occurs primarily in joints where leukocyte infiltration results in synovitis and synovium thickening producing the typical symptoms of warmth, redness, swelling and pain. As the disease progresses, the aberrant collection of cells invade and destroy the cartilage and bone within the joint leading to deformities and chronic pain. The inflammatory cytokines TNFα, IL-1β and IL-8 act as critical mediators of this infiltration, and these cytokines are present in the synovial fluid of patients with RA.

Leukocytes localize to sites of inflammatory injury as a result of the integrated actions of adhesion molecules, cytokines, and chemotactic factors. In lipopolysaccharide-induced arthritis in the rabbit, the production of TNFα, and IL-1β in the initiative phase of inflammation paralleled the time course of leukocyte infiltration. The adherence of neutrophils to the vascular endothelium is a first step in the extravasation of cells into the interstitium. This process is mediated by selectins, integrins, and endothelial adhesion molecules, e.g. ICAM-1 and VCAM-1. Since TNFα induces the expression of ICAM-1 and VCAM-1 and is present in high concentrations in arthritic joints, it is likely that this protein plays a central role in the pathogenesis of the disease. This is supported by the clinical activity of anti-TNFα therapies such as Remicade. After adherence to the endothelium, leukocytes migrate along a chemoattractant concentration gradient. A further critical process in the progression of RA is the enhancement of the blood supply to the synovium through angiogenesis. Expression of the key angiogenic factor VEGF is potently induced by pro-inflammatory cytokines including TNFα. Together, these data point to important roles of TNFα, leukocytes, leukocyte adhesion molecules, leukocyte chemoattractants and angiogenesis in the pathogenesis of arthritic injury.

Early in the disease, immunologic reactions or other activating signals promote the release of inflammatory cytokines, particularly TNFα and IL-1β from macrophages and mast cells. Ceramide is produced by the hydrolysis of sphingomyelin in response to inflammatory stresses, including TNFα and IL-1β(Dressler et al., 1992, Science 255: 1715). Ceramide can be further hydrolyzed by ceramidase to produce sphingosine which is then rapidly phosphorylated by SK to produce S1P. Ceramidase and SK are also activated by cytokines and growth factors, leading to rapid increases in the intracellular levels of S1P and depletion of ceramide levels. This situation promotes cell proliferation and inhibits apoptosis. Deregulation of apoptosis in phagocytes is an important component of the chronic inflammatory state in arthritis, and S1P has been shown to protect neutrophils from apoptosis in response to Fas, TNFα and ceramide. Similarly, apoptosis of macrophages is blocked by S1P.

In addition to its role in regulating cell proliferation and apoptosis, S1P is a central player in the pathway since it has pleiotropic actions on the endothelial cells, leukocytes, chondrocytes and synovial cells. Within the endothelial cells, S1P activates NFκB thereby inducing the expression of multiple adhesion molecules and COX-2 resulting in PGE₂ synthesis. Together, this chemoattractant and the adhesion molecules promote neutrophil infiltration into the synovium. At the same time, S1P directly activates the neutrophils resulting in the release of oxygen free radicals that destroy joint tissue. Progression of RA is associated with a change from a Th1 to a Th2 environment, and sphingosine is selectively inhibitory toward Th1 cells. Consequently, inhibiting the conversion of sphingosine to S1P should attenuate the progression of the disease. Platelets, monocytes and mast cells secrete S1P upon activation, promoting inflammatory cascades at the site of tissue damage(Yatomi et al., 1995, Blood 86: 193). S1P also promotes the secretion of proteases from chondrocytes that contribute to joint destruction. Finally, S1P-mediated expression of VEGF promotes the angiogenesis necessary to support the hyperproliferation of synovial cells. Consequently, inhibiting the conversion of sphingosine to S1P should attenuate the progression of the disease.

As the processes involved in arthritis are induced by cytokines and growth factors whose action is dependent on increased signaling through SK, the SK inhibitory compounds, pharmaceutical compositions and methods of this invention are expected to be of use in the prevention and/or therapy of arthritis.

Atherosclerosis is a complex vascular disease that involves a series of coordinated cellular and molecular events characteristic of inflammatory reactions In response to vascular injury, the first atherosclerotic lesions are initiated by acute inflammatory reactions, mostly mediated by monocytes, platelets and T lymphocytes. These inflammatory cells are activated and recruited into the subendothelial vascular space through locally expressed chemotactic factors and adhesion molecules expressed on endothelial cell surface. Continuous recruitment of additional circulating inflammatory cells into the injured vascular wall potentiates the inflammatory reaction by further activating vascular smooth muscle (VSM) cell migration and proliferation. This chronic vascular inflammatory reaction leads to fibrous cap formation, which is an oxidant-rich inflammatory milieu composed of monocytes/macrophages and VSM cells. Over time, this fibrous cap can be destabilized and ruptured by extracellular metalloproteinases secreted by resident monocytes/macrophages. The ruptured fibrous cap can easily occlude vessels resulting in acute cardiac or cerebral ischemia. This underlying mechanism of atherosclerosis indicates that activation of monocyte/macrophage and VSM cell migration and proliferation play critical roles in the development and progression of atherosclerotic lesions. Importantly, it also suggests that a therapeutic approach that blocks the activities of these vascular inflammatory cells or smooth muscle cell proliferation should be able to prevent the progression and/or development of atherosclerosis.

SK is highly expressed in platelets allowing them to phosphorylate circulating sphingosine to produce S1P. In response to vessel injury, platelets release large amounts of S1P into the sites of injury which can exert mitogenic effects on VSM cells by activating S1P receptors. S1P is also produced in activated endothelial and VSM cells. In these cells, intracellularly produced S1P functions as a second messenger molecule, regulating Ca²⁺ homeostasis associated with cell proliferation and suppression of apoptosis. Additionally, deregulation of apoptosis in phagocytes is an important component of the chronic inflammatory state of atherosclerosis, and S1P protects granulocytes from apoptosis, Together, these studies indicate that activation of SK alters sphingolipid metabolism in favor of S1P formation, resulting in pro-inflammatory and hyper-proliferative cellular responses.

In addition to its role in regulating cell proliferation and apoptosis, S1P has been shown to have several important effects on cells that mediate immune functions. Platelets and monocytes secrete cytokines, growth factors and S1P upon activation, promoting inflammatory cascades at the site of tissue damage. For example, TNFα has been shown to act through the induction of nuclear factor kappa B (NFκB), which has been implicated in increasing the proinflammatory enzymes nitric oxide synthase (NOS) and cyclooxygenase-2 (COX-2). COX-2 may play a key role in the inflammation of atherosclerosis through its production of prostaglandins, and oxidative stress such as that mediated by nitric oxide produced by NOS has also shown to exacerbate inflammation. Activation of SK is required for signaling responses since the ability of inflammatory cytokines to induce adhesion molecule expression via activation of NFκB is mimicked by S1P. Similarly, S1P mimics the ability of TNFα to induce the expression of COX-2 and the synthesis of PGE₂, and knock-down of SK by RNA interference blocks these responses to TNFα but not S1P. S1P is also a mediator of Ca²⁺ influx during granulocyte activation, leading to the production of superoxide and other toxic radicals.

Together, these studies indicate that SK is a new molecular target for atherosclerosis. The use of inhibitors of SK as anti-atherosclerosis agents will prevent the deleterious activation of leukocytes, as well as prevent infiltration and smooth muscle cell hyperproliferation, making the compounds, pharmaceutical compositions and methods of this invention useful for the treatment and/or prevention of atherosclerosis.

The physiological endpoint in asthma pathology is narrowing of the bronchial tubes due to inflammation. In a large portion of asthma cases, the inflammation is initiated and later amplified by exposure to allergens. Upon inhalation, these allergens, bind to circulating IgE and then bind to the high-affinity FcεRI surface receptors expressed by inflammatory cells residing in the bronchial mucosa. This extracellular binding leads to a cascade of signaling events inside the inflammatory cells, culminating in activation of these cells and secretion of multiple factors that trigger the cells lining the bronchial airways to swell, resulting in restricted bronchial tubes and decreased air exchange. The inflammation process in response to the initial exposure to allergen may not completely subside. Furthermore, additional exposures may lead to an exaggerated response called bronchial hyper-reactivity. This hyper-reactive state can lead to a permanent condition of restricted airways through airway remodeling Consequently, unchecked inflammatory responses to initial allergen exposure may result in chronic inflammation and permanent bronchiolar constriction Therefore, inhibiting or diminishing this exaggerated inflammation would likely decrease the symptoms associated with asthma.

Many studies have revealed the involvement of mast cells in the inflammatory process leading to asthma, and SK has been shown to be involved in allergen-stimulated mast cell activation, a critical step in the bronchial inflammatory process In rat basophilic leukemia RBL-2H3 cells, IgE/Ag binding to the high-affinity FcεR1 receptor leads to SK activation and conversion of sphingosine to S1P (Choi et al., 1996, Nature 380: 634). The newly formed S1P increases intracellular calcium levels, which is necessary for mast cell activiation. Alternately, high concentrations of sphingosine decrease IgE/Ag exposure-mediated leukotriene synthesis and diminishe cytokine transcription and secretion (Prieschl et al., 1999, J Exp Med 900:1).

In addition to the key role of SK and S1P in mast cell activation, S1P also has direct effects on downstream signaling in the asthma inflammation pathway. Ammit and coworkers demonstrated increased S1P levels in bronchoalveolar lavage (BAL) fluid collected from asthmatic patients 24 hours after allergen challenge compared with non-asthmatic subjects (Ammit et al., 2001, FASEB J 15: 1212). In conjunction with the finding that activated mast cells produce and secrete S1P, these results reveal a correlation between S1P and the asthmatic inflammatory response. To evaluate a possible role of SK and S1P exposure to cell response, ASM cultures were grown in the presence of S1P (Ammit et al., 2001, FASEB J 15: 1212). Furthermore, airway smooth muscle (ASM) cells are responsive to S1P- and SK-dependent stimuli, such as TNFα and IL-1β. Treatment with S1P increases phosphoinositide hydrolysis and intracellular calcium mobilization, both of which promote ASM contraction Furthermore, S1P treatment increases DNA synthesis, cell number and accelerated progression of ASM cells from G₁ to S phase.

In addition to the direct effects on ASM cells, S1P also regulates secretion of cytokines and expression of cell adhesion molecules that amplify the inflammatory response through leukocyte recruitment and facilitating extracellular component interaction S1P, like TNFα, induces IL-6 secretion and increases the expression of cell adhesion molecules such as VCAM-1, ICAM-1 and E-selectin (Shimamura et al., 2004, Eur J Pharmacol 486: 141). In addition to the effects of S1P on mast cell activation, the multiple roles of S1P, and hence SK, in the bronchiolar inflammatory phase of asthma pathogenesis clearly indicate an opportunity for pharmacologic intervention in both the acute and chronic phases of this disease.

Overall, SK is a target for new anti-asthma therapies. The use of inhibitors of SK as anti-asthma agents will inhibit cytokine-mediated activation of leukocytes, thereby preventing the deleterious activation of leukocytes, as well as preventing airway smooth muscle cell hyperproliferation, making the compounds, pharmaceutical compositions and methods of this invention useful for the treatment and/or prevention of asthma.

Chronic obstructive pulmonary disease (COPD), like asthma, involves airflow obstruction and hyperresponsiveness that is associated with aberrant neutrophil activation in the lung tissue. This is clinically manifested as chronic bronchitis, fibrosis or emphysema, which together make up the fourth leading cause of death in the United States. Since activation of inflammatory cells by chemical insults in COPD occurs through NFκB-mediated pathways similar to those activated during asthma, it is likely that the compounds, pharmaceutical compositions and methods of this invention will also be useful for the treatment and/or prevention of COPED.

Inflammation is involved in a variety of skin disorders, including psoriasis, atopic dermatitis, contact sensitivity and acne, which affect more than 20% if the population. Although topical corticosteroids have been widely used, their adverse effects prevent long-term use. Since the inflammatory responses typically involve aberrant activation of signaling pathways detailed above, it is likely that the compounds, pharmaceutical compositions and methods of this invention will also be useful for the treatment of these skin diseases.

A variety of diseases including allergic encephalomyclitis, allergic neuritis, transplant allograft rejection, graft versus host disease, myocarditis, thyroiditis, nephritis, systemic lupus erythematosus, and insulin-dependent diabetes mellitus can be induced by inappropriate activation of T cells. Common features of the pathogenesis of these diseases include infiltration by mononuclear cells, expression of CD4 and CD8 autoreactive T cells, and hyperactive signaling by inflammatory mediators such as IL-1, IL-6 and TNFα. Since the inflammatory responses typically involve aberrant activation of signaling pathways detailed above, it is likely that the compounds, pharmaceutical compositions and methods of this invention will also be useful for the treatment of these T cell-mediated diseases of immunity.

Angiogenic Disease.

The present invention also relates to compounds, pharmaceutical compositions and methods useful for the treatment and/or prevention of diseases that involve undesired angiogenesis. More specifically, the invention relates to the use of chemical compounds and compositions that inhibit the enzymatic activity of sphingosine kinase for the treatment and/or prevention of angiogenic diseases, such as diabetic retinopathy, arthritis, cancer, psoriasis, Kaposi's sarcoma, hemangiomas, myocardial angiogenesis, atherosclerotic plaque neovascularization, and ocular angiogenic diseases such as choroidal neovascularization, retinopathy of prematurity (retrolental fibroplasias), macular degeneration, corneal graft rejection, rubeosis, neuroscular glacoma and Oster Webber syndrome. The following discussion demonstrates the role of SK in several of these angiogenic diseases. Since the same processes are involved in the above listed diseases, the compounds, pharmaceutical compositions and methods of this invention will be useful for the treatment and/or prevention of a variety of diseases.

Angiogenesis refers to the state in the body in which various growth factors or other stimuli promote the formation of new blood vessels. As discussed below, this process is critical to the pathology of a variety of diseases. In each case, excessive angiogenesis allows the progression of the disease and/or the produces undesired effects in the patient. Since conserved biochemical mechanisms regulate the proliferation of vascular endothelial cells that form these new blood vessels, i.e. neovascularization, identification of methods to inhibit these mechanisms are expected to have utility for the treatment and/or prevention of a variety of diseases. The following discussion provides further details in how the compounds, compositions and methods of the present invention can be used to inhibit angiogenesis in several of these diseases.

Diabetic retinopathy is a leading cause of vision impairment, and elevation in the expression of growth factors contributes to pathogenic angiogenesis in this disease. In particular, vascular endothelial growth factor (VEGF) is a prominent contributor to the new vessel formation in the diabetic retina (Frank et al., 1997, Arch Ophthalmol 115: 1036). Sone et al., 1997, Diabetologia 40: 726), and VEGF has been shown to be elevated in patients with proliferative diabetic retinopathy (Aiello et al., 1994, N Engl J Med 331: 1480). In addition to diabetic retinopathy, several other debilitating ocular diseases, including age-related macular degeneration and choroidal neovascularization, are associated with excessive angiogenesis that is mediated by VEGF and other growth factors (Grant et al., 2004, Expert Opin Investig Drugs 13: 1275).

In the retina, VEGF is expressed in the pigmented epithelium, the neurosensory retina, the pericytes and the vascular smooth muscle layer. VEGF induces endothelial cell proliferation, favoring the formation of new vessels in the retina (Pe'er et al., 1995, Lab Invest 72: 638). At the same time, basic fibroblast growth factor (bFGF) in the retina is activated, and this factor acts in synergy with VEGF such that the two together induce the formation of new vessels in which the subendothelial matrix is much weaker than in normal vessels. Additionally, VEGF facilitates fluid extravasation in the interstitium, where exudates form in the retinal tissue. VEGF also promotes the fenestration of endothelial cells, a process that can give rise to intercellular channels through which fluids can leak, and disrupts tight junctions between cells. Thus, reduction of VEGF activity in the retina is likely to efficiently reduce the development and progression of retinal angiogenesis and vascular leakage which underlie the retinopathic process.

The pro-inflammatory cytokine TNFα has also been demonstrated to play a role in diabetic retinopathy since it alters the cytoskeleton of endothelial cells, resulting in leaky barrier function and endothelial cell activation (Camussi et al., 1991, Int Arch Allergy Appl Immunol 96: 84). These changes in retinal endothelial cells are central in the pathologies of diabetic retinopathy,

A link between the actions of VEGF and SK may be involved in driving retinopathy.

SK has been shown to mediate VEGF-induced activation of ras- and mitogen-activated protein kinases (Shu et al., 2002, Mol Cell Biol 22: 7758). VEGF has been shown to enhance intracellular signaling responses to S1P, thereby increasing its angiogenic actions (Igarashi et al., 2003, Proc Natl Acad Sci USA 100: 10664). S1P has also been shown to stimulate NFκB activity (Xia et al., 1998, Proc Natl Acad Sci USA 95: 14196) leading to the production of COX-2, adhesion molecules and additional VEGF production, all of which have been linked to angiogenesis. Furthermore, the expression of the endothelial isoform of nitric oxide synthase (eNOS), a key signaling molecule in vascular endothelial cells and modulates a wide array of function including angiogenic responses, is regulated by SK (Igarashi et al., 2000, J Biol Chem 275: 32363). Clearly, SK is a central regulator of angiogenesis, supporting our hypothesis that its pharmacological manipulation may be therapeutically useful. S1P has also been shown to stimulate NFκB production which has been demonstrated to be angiogenic. NFκB leads to the production of Cox2, adhesion molecules and additional VEGF production, all of which have been linked to angiogenesis.

One of the most attractive sites of intervention in this pathway is the conversion of sphingosine to S1P by the enzyme SK. SK is the key enzyme responsible for the production of S1P synthesis in mammalian cells, which facilitates cell survival and proliferation, and mediates critical processes involved in angiogenesis and inflammation, including responses to VEGF (Shu et al., 2002, Mol Cell Biol 22: 7758) and TNFα (Xia et al., 1998, Proc Natl Acad Sci USA 95: 14196). Therefore, inhibition of S1P production is a potentially important point of therapeutic intervention for diabetic retinopathy.

The role of angiogenesis in cancer is well recognized. Growth of a tumor, is dependent on neovascularization so that nutrients can be provided to the tumor cells. The major factor that promotes endothelial cell proliferation during tumor neovascularization is VEGF. As discussed above, signaling through VEGF receptors is dependent on the actions of SK. Therefore, the compounds, pharmaceutical compositions and methods of this invention will have utility for the treatment of cancer.

More than 50 eye diseases have been linked to the formation of choroidal neovascularization, although the three main diseases that cause this pathology are age-related macular degeneration, myopia and ocular trauma. Even though most of these causes are idiopathic, among the known causes are related to degeneration, infections, choroidal tumors and or trauma. Among soft contact lens wearers, choroidal neovascularization can be caused by the lack of oxygen to the eyeball. As the choroidal neovascularization is induced by growth factors whose action is dependent on increased signaling through SK, the SK inhibitory compounds, pharmaceutical compositions and methods of this invention are expected to be of use in the therapy of disorders of choroidal neovascularization.

Hemangiomas are angiogenic diseases characterized by the proliferation of capillary endothelium with accumulation of mast cells, fibroblasts and macrophages. They represent the most frequent tumors of infancy, and are characterized by rapid neonatal growth (proliferating phase). By the age of 6 to 10 months, the hemangiomas growth rate becomes proportional to the growth rate of the child, followed by a very slow regression for the next 5 to 8 years (involuting phase). Most hemangiomas occur as single tumors, whereas about 20% of the affected infants have multiple tumors, which may appear at any body site. Several studies have provided insight into the histopathology of these lesions. In particular, proliferating hemangiomas express high levels of proliferating cell nuclear, antigen (a market, for cells in the S phase), type IV collagenase, VEGF and FGF-2. As the hemangiomas are induced by growth factors whose action is dependent on increased signaling through SK, the SK inhibitory compounds, pharmaceutical compositions and methods of this invention are expected to be of use in the therapy of hemangiomas.

Psoriasis and Kaposi's sarcoma are angiogenic and proliferative disorders of the skin. Hypervascular psoriatic lesions express high levels of the angiogenic inducer IL-8, whereas the expression of the endogenous inhibitor TSP-1 is decreased. Kaposi's sarcoma (KS) is the most common tumor associated with human immunodeficiency virus (HIV) infection and is in this setting almost always associated with infection by human herpes virus 8. Typical features of KS are proliferating spindle-shaped cells, considered to be the tumor cells and endothelial cells forming blood vessels. KS is a cytokine-mediated disease, highly responsive to different inflammatory mediators like IL-1β, TNF-α and IFN-γ and angiogenic factors. As the progression of psoriasis and KS are induced by growth factors whose action is dependent on increased signaling through SK, the SK inhibitory compounds, pharmaceutical compositions and methods of this invention are expected to be of use in the therapy of these disorders.

### EXAMPLES

The present invention may be better understood with reference to the following examples. These examples are intended to be representative of specific embodiments of the invention, and are not intended as limiting the scope of the invention.

Representative compounds of the invention include those in Tables 1, 2 and 3. Structures were named using Chemdraw Ultra, version 7.0.1, available from CambridgeSoft Corporation, 100 CambridgePark Drive, Cambridge, Mass. 02140, USA

TABLE 1
Representative compounds of the invention.

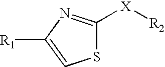

| # | X | R₁ | R₂ | Chemical name |
|---|---|---|---|---|
| 1 | 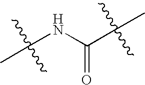 | 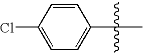 | 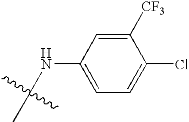 | 1-[4-(4-Chloro-phenyl)- thiazol-2-yl]-3-(4-chloro- 3-trifluoromethyl- phenyl)-urea |
| 2 | 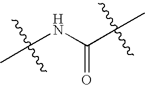 | 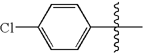 | 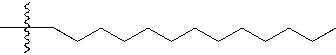 | Tetradecanoic acid[4- (4-chloro-phenyl)- thiazol-2-yl]-amide |
| 3 | 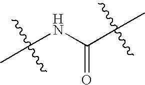 | 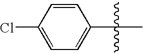 | 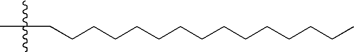 | Hexadecanoic acid[4- (4-chloro-phenyl)- thiazol-2-yl)-amide |
| 4 | 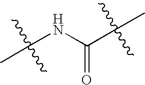 | 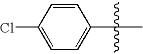 | 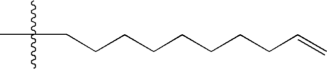 | Undec-10-enoic acid- [4-(4-chloro-phenyl)- thiazol-2-yl]-amide |
| 5 | 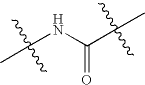 | 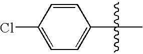 | 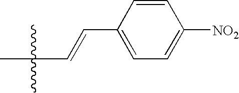 | N-[4-(4-Chloro- phenyl)-thiazol-2-yl]-3- (4-nitro-phenyl)- acrylamide |
| 6 | 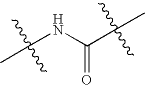 | 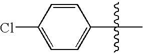 | 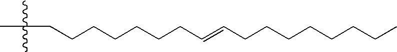 | Octadec-9-enoic acid- [4-(4-chloro-phenyl)- thiazol-2-yl]-amide |
| 7 | 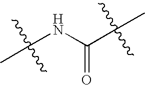 | 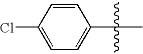 | 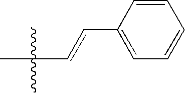 | N-[4-(4-Chloro- phenyl)-thiazo]-2-yl]-3- phenyl-acrylamide |
| 8 | 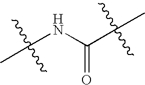 | 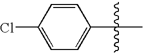 | 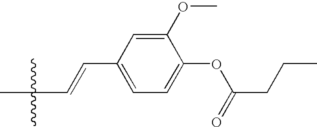 | Butyric acid 4-{2-[4-(4- chloro-phenyl)-thiazol- 2-ylcarbamoyl]-vinyl}- 2-methoxy-phenyl ester |
| 9 | 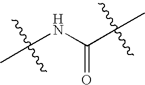 | 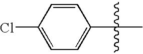 | 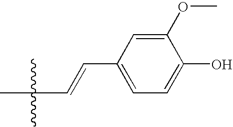 | N-[4-(3-Chloro- phenyl)-thiazol-2-yl]-3- (4-hydroxy-3-methoxy- phenyl)-acrylamide |
| 10 | 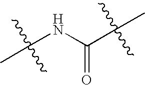 | 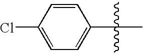 | 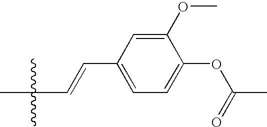 | Acetic acid 4-(2-[4-(4- chloro-phenyl)-thiazol- 2-ylcarbamoyl]-vinyl}- 2-methoxy-phenyl ester |
| 11 | 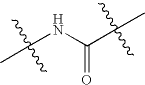 | 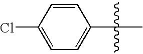 | 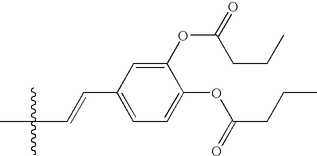 | Butyric acid 2- butyryloxy-5-{2-[4-(4- chloro-phenyl)-thiazol- 2-ylcaxbamoyl]-vinyl}- phenyl ester |
| 12 | 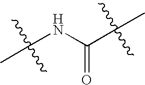 | 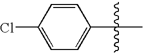 | 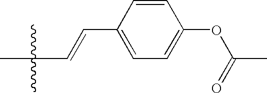 | Acetic acid 4-{2-[4-(4- chloro-phenyl)-thiazol- 2-ylcaxbamoyl]-vinyl}- phenyl ester |
| 13 | 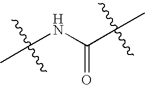 | 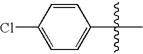 | 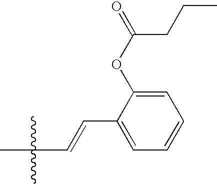 | Butyric acid 2-{2-[4-(4- chloro-phenyl)-thiazol- 2-ylcarbamoyl]-vinyl}- phenyl ester |
| 14 | 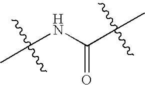 | 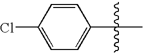 | 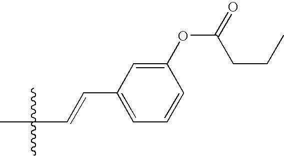 | Butyric acid 3-{2-[4-(4- chloro-phenyl)-thiazol- 2-ylcarbamoyl]-vinyl}- phenyl ester |
| 15 | 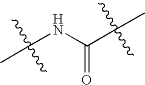 | 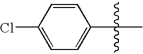 | 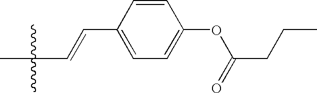 | Butync acid 4-{2-[4-(4- chloro-phenyl)-thiazol- 2-ylcarbamoyl]-vinyl}- phenyl ester |
| 16 | 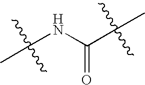 | 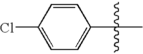 | 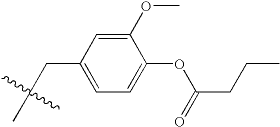 | Butyric acid 4-{[4-(4- chloro-phenyl)-thiazol- 2-ylcarbamoyl]- methyl}-2-methoxy- phenyl ester |
| 17 | 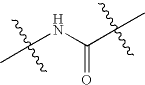 | 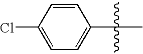 | 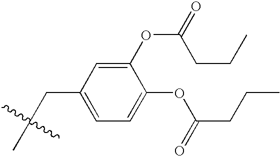 | Butyric acid 2- butyryloxy-5-{[4-(4- chloro-phenyl)-thiazol- 2-ylcarbamoyl]- methyl}-phenyl ester |
| 18 | 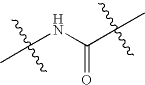 | 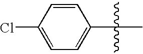 | 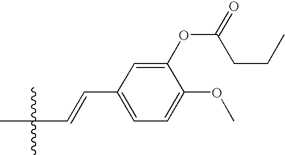 | Butyric acid 5-{2-[4-(4- chloro-phenyl)-thiazol- 2-ylcarbamoyl]-vinyl}- 2-methoxy-phenyl ester |
| 19 | 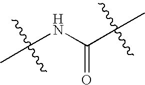 | 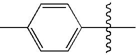 | 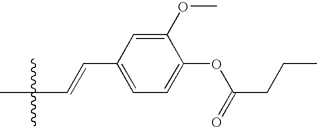 | Butyric acid 2- methoxy-4-[2-(4-p- tolyl-thiazol-2- ylcarbamoyl)-vinyl]- phenyl ester |
| 20 | 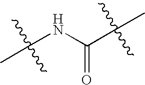 | 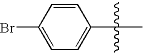 | 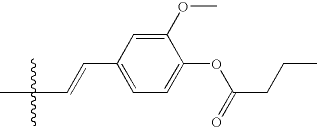 | Butyric acid 4-{2-[4-(4- bromo-phenyl)-thiazol- 2-ylcarbamoyl]-vinyl}- 2-methoxy-phenyl ester |
| 21 | 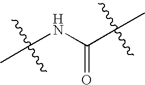 | 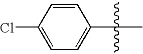 | 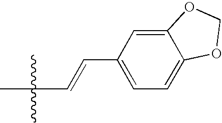 | 3-Benzo[1,3]dioxol-5- yl-N-[4-(4-chloro- pheny])-thiazol-2-yl]- acrylamide |
| 22 | 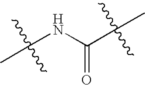 | 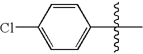 | 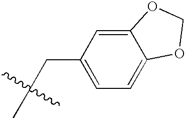 | 2-Benzo[1,3]dioxol-5- y]-N-[4-(4-chloro- phenyl)-thiazol-2-yl]- acetamide |
| 23 | 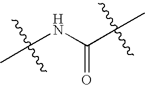 | 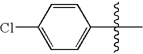 | 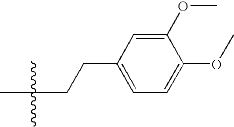 | N-[4-(4-Chloro- phenyl)-thiazol-2-yl]-3- (3,4-dimethoxy- phenyl)-propionamide |
| 24 | 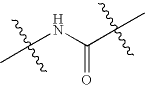 | 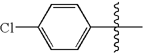 | 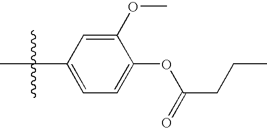 | Butyric acid 4-[4-(4- chloro-phenyl)-thiazol- 2-ylcarbamoyl]-2- methoxy-phenyl ester |
| 25 | 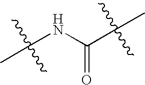 | 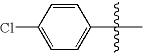 | 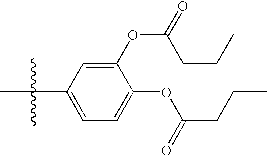 | Butyric acid 2- butyryloxy-4-[4-(4- chloro-phenyl)-thiazo[- 2-ylearbamoyl]-phenyl |
| 26 | 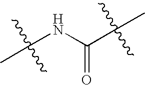 | 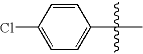 | 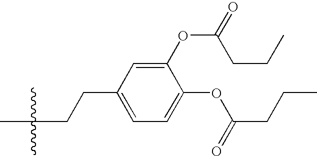 | Butyric acid 2- butyryloxy-4-{2-[4-(4- chloro-phenyl)-thiazol- 2-ylcarbamoyl]-ethyl}- phenyl ester |
| 27 | 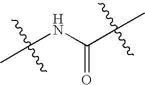 | 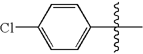 | 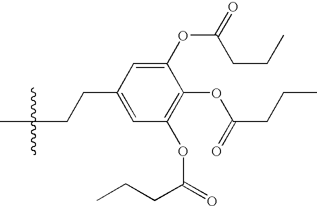 | Butyric acid 2,6-bis- butyryloxy-4-[4-(4- chloro-phenyl)-thiazol- 2-ylcarbamoyl]-phenyl ester |
| 28 | 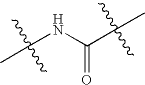 | 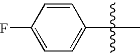 | 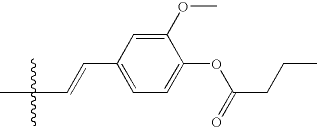 | Butyric acid 4-{2-[4-(4- fluoro-phenyl)-thiazol- 2-ylcarbamoyl]-vinyl}- 2-methoxy-phenyl ester |
| 29 | 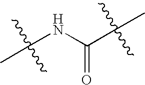 | 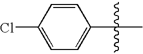 | 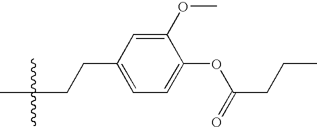 | Butyric acid 4-{2-[4-(4- chloro-phenyl)-thiazol- 2-ylcarbamoyl]-ethyl}- 2-methoxy-phenyl ester |
| 30 | 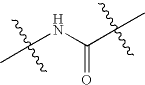 | 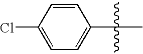 | 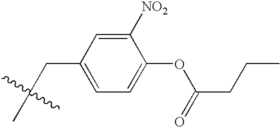 | Butyric acid 4-{[4-(4- chloro-phenyl)-thiazol- 2-ylcarbamoyl]- methyl}-2-nitro-phenyl ester |
| 31 | 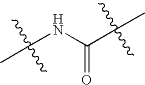 | 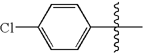 | 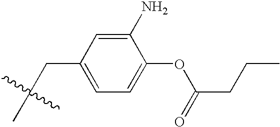 | Butyric acid 2-amino-4- {[4-(4-chloro-phenyl)- thiazol-2- ylcarbamoyl]-methyl}- phenyl ester |
| 32 | 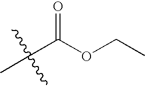 | 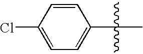 | | 4-(4-Chloro-phenyl)- thiazole-2-carboxylic acid ethyl ester |
| 33 | 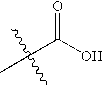 | 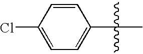 | | 4-(4-Chloro-phenyl)- thiazole-2-carboxylic acid |
| 34 | 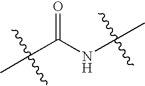 | 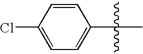 | 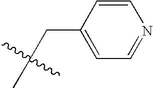 | 4-(4-Chloro- phenyl)thiazole-2- carboxylic acid (pyridin-4- ylmethyl)amide |
| 35 | 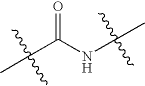 | 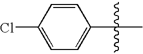 | 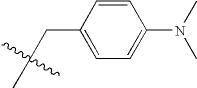 | 4-(4-Chloro-phenyl)- thiazole-2-carboxylic acid 4-dimethylamino- benzylamide |
| 36 | 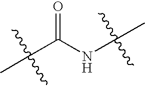 | 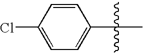 | 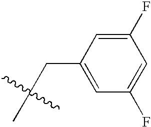 | 4-(4-Chloro-phenyl)- thiazole-2-carboxylic acid 3,5-difluoro- benzylamide |
| 37 | 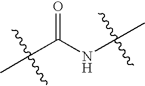 | 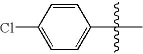 | 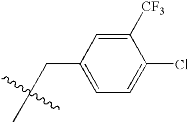 | 4-(4-Chloro-phenyl)- thiazole-2-carboxylic acid 4-chloro-3- trifluoromethyl- benzylamide |
| 38 | 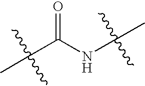 | 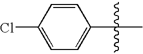 | 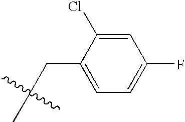 | 4-{4-Chloro-pbenyl)- thiazole-2-carboxylic acid 2-chloro-4-fluoro- benzylamide |
| 39 | 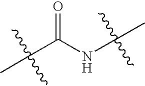 | 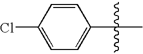 | 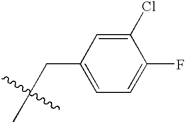 | 4-(4-Chloro-phenyl)- thiazole-2-carboxylic acid 3-chloro-4-fluoro- benzylamide |
| 40 | 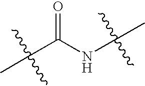 | 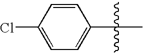 | 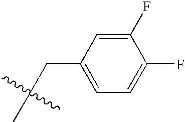 | 4-(4-Chloro-phenyl)- thiazole-2-carboxylic acid 3,4-difluoro- benzylamide |
| 41 | 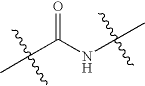 | 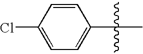 | 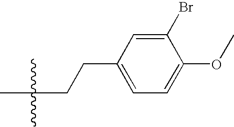 | 4-(4-Chloro-phenyl)- thiazole-2-carboxylic acid[2-(3-bromo-4- methoxy-pbenyl)- ethyl]-amide |
| 42 | 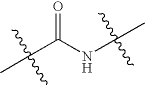 | 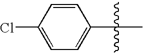 | 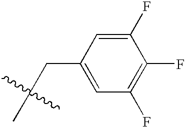 | 4-(4-Chloro-phenyl)- thiazole-2-carboxylic acid 3,4,5-trifluoro- benzylamide |
| 43 | 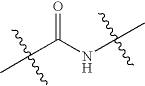 | 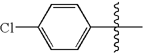 | 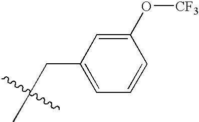 | 4-(4-Chloro-phenyl)- thiazole-2-carboxylic acid 3- trifluoromethoxy benzylamide |
| 44 | 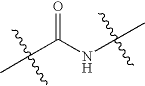 | 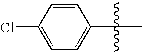 | 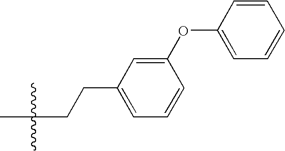 | 4-(4-Chloro- phenyl)-thiazole-2- carboxylic acid [2-(3- phenoxy-phenyl) ethyl]-amide |
| 45 | 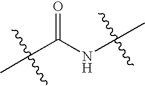 | 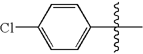 | 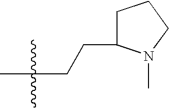 | 4-(4-Chloro-phenyl)- thiazole-2-carboxylic acid[2-(1-methyl- pyrrolidin-2-yl)-ethyl]- amide |
| 46 | 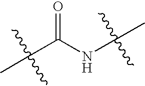 | 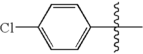 | 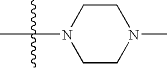 | 4-(4-Chloro-phenyl)- thiazole-2-carboxylic acid (4-methyl- piperazin-1-yl)-amide |
| 47 | 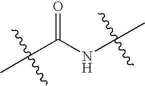 | 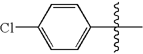 | 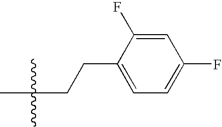 | N-[4-(4-Chloro- phenyl)-thiazol-2-yl)-3- (2,4-difluoro-phenyl)- propionamide |
| 48 | 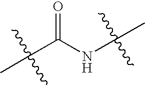 | 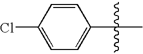 | 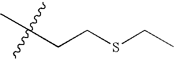 | 4-(4-Chloro-phenyl)- thiazole-2-carboxylic acid (2-ethylsulfanyl- ethyl)-amide |
| 49 | 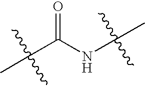 | 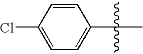 | 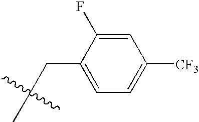 | 4-(4-Chloro-phenyl)- thiazole-2-carboxylic acid 2-fluoro-4- trifluoromethyl- benzylamide |
| 50 | 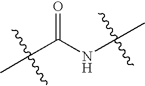 | 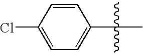 | 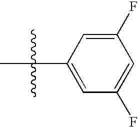 | 4-(4-Chloro-phenyl)- thiazol-2-carboxylic acid (3,5-difluoro- phenyl)-amide |
| 51 | 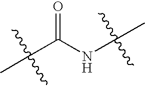 | 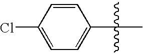 | 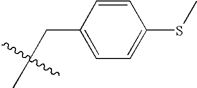 | 4-(4-Chloro- phenyl)-thiazole-2- carboxylic acid 4- methylsulfanyl- benzylamide |
| 52 | 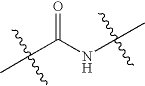 | 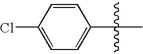 | 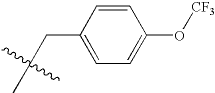 | 4-(4-Chloro-phenyl)- thiaizole-2-carboxylic acid 4- trifluoromethoxy- benzyl amide |
| 53 | 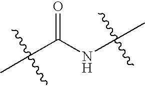 | 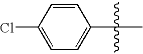 | 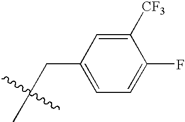 | 4-{4-Chloro-phenyl)- thiazole-2-carboxylic acid 4-fluoro-3- trifluoromethyl- benzylamide |
| 54 | 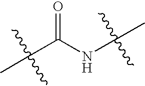 | 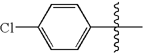 | 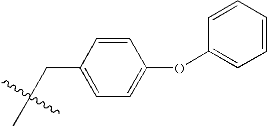 | 4-(4-Chloro-phenyl)- thiazole-2-carboxylic acid 4-phenoxy- benaylamide |
| 55 | 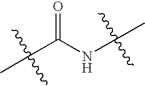 | 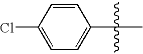 | 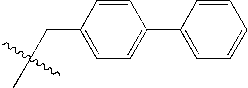 | 4-(4-Chloro-phenyl)- thiazole-2-carboxylic acid (biphenyl-4- ylmethyl)-amide |
| 56 | 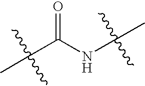 | 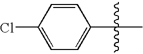 | 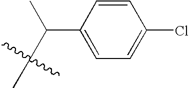 | 4-(4-Chloro-phenyl)- thiazole-2-carboxylic acid[1-(4-chloro- phenyl)-ethyl]-amide |
| 57 | 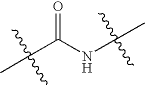 | 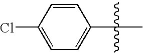 | 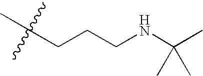 | 4-(4-Chloro-phenyl)- thiazole-2-carboxylic acid (3-tert-butylamino- propyl)-amide |
| 58 | 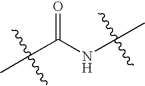 | 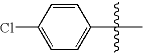 | 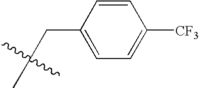 | 4-(4-Chloro-phenyl)- thiazole-2-carboxylic acid 4-trifluoromethyl- benzylamide |
| 59 | 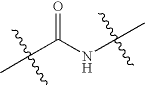 | 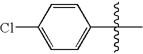 | 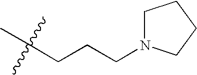 | 4-(4-Chloro-phenyl)- thiazole-2-carboxylic acid (3-pyrrolidin-1-yl- propyl)-amide |
| 60 | 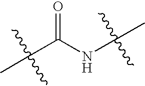 | 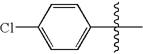 | 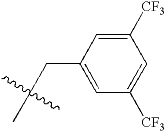 | 4-(4-Chloro-phenyl)- thiazole-2-carboxylic acid 3,5-bis- trifluoromethyl benzylamide |
| 61 | 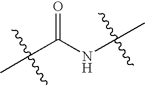 | 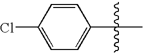 | 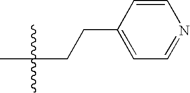 | 4-(4-Chloro-phenyl)- thiazole-2-carboxylic acid (2-pyridin-4-yl- ethyl)-amide |
| 62 | 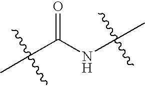 | 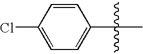 | 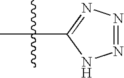 | 4-(4-Chloro-phenyl)- thiazole-2-carboxylic acid (1H-tetrazol-5-yl)- amide |
| 63 | 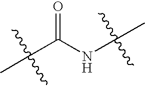 | 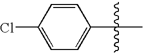 | 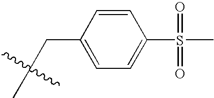 | 4-(4-Chloro-phenyl)- thiazole-2-carboxylic acid 4- methanesulfonyl- benzylamide |
| 64 | 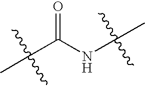 | 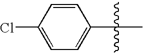 | 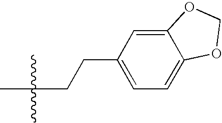 | 4-(4-Chloro-phenyl)- thiazole-2-carboxylic acid (2- benzo[1,3]dioxol-5-yl- ethyl)-amide |
| 65 | 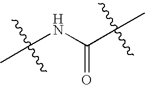 | 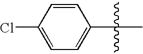 | 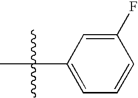 | N-[4-(4-Chloro- phenyl)-thiazol-2-yl]-3- fluoro-benzamide |
| 66 | 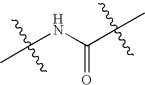 | 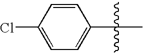 | 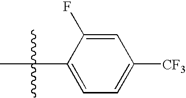 | N-[4-(4-Chloro- phenyl)-thiazol-2-yl]-2- fluoro-4- trifluoromethyl benzamide |
| 67 | 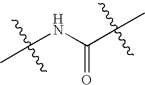 | 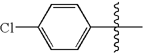 | 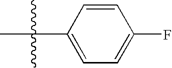 | N-[4-(4-Chloro- phenyl)-thiazol-2-yl]-4- fluoro-benzamide |
| 68 | 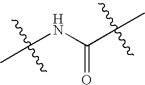 | 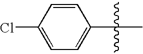 | 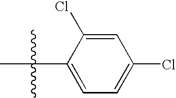 | 2,4-Dichloro-N-[4-(4- chloro-phenyl)-thiazol- 2-yl]-benzamide |
| 69 | 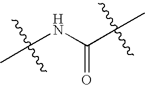 | 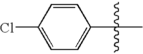 | 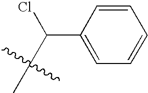 | 2-Chloro-N-[4-(4- chloro-phenyl)-thiazol- 2-yl]-2-phenyl- acetamide |
| 70 | 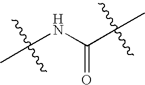 | 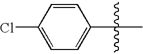 | 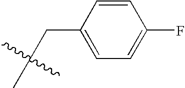 | N-[4-(4-Chloro- phenyl)-thiazol-2-yl]-2- (4-fluoro-phenyl)- acetamide |
| 71 | 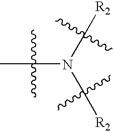 | 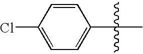 | 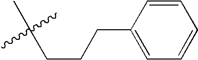 | [4-(4-Chloro-phenyl)- thiazol-2-yl]-bis-(3- phenyl-propyl)-amine |
| 72 | 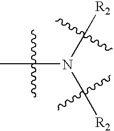 | 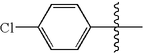 | 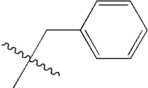 | Dibenzyl-[4-(4-chloro- phenyl)-thiazol-2-yl]- amine |
| 73 | 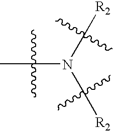 | 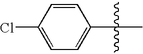 | 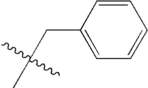 | Benzyl-[4-(4-chloro- phenyl)-thiazol-2-yl]- amine |
| 74 | 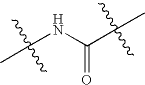 | 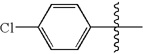 | 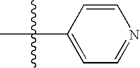 | 4-(4-Chloro-phenyl)- thiazole-2-carboxylic acid (2-pyridin-4-yl)- amide |
| 75 | 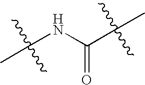 | 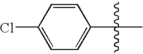 | 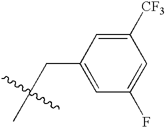 | 4-(4-Chloro-phenyl)- thiazole-2-carboxylic acid 3-fluoro-5- trifluoromethyl benzylamide |
| 76 | 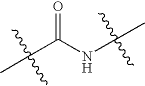 | 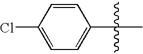 | 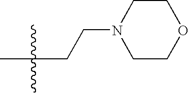 | 4-(4-Chloro-phenyl)- thiazole-2-carboxylic acid (2-morpholin-4-yl- ethyl)-amide |
| 77 | 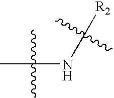 | 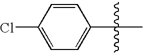 | 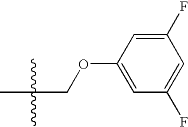 | [4-(4-Chloro-phenyl)- thiazol-2-yl]-(3,5- difluoro- phenoxymethyl)-amine |
| 78 | 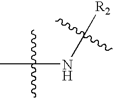 | 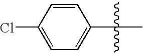 | 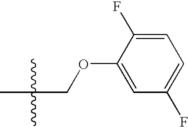 | [4-(4-Chloro-phenyl)- thiazol-2-yl]-(2,5- difluoro- phenoxymethyl)-amino |
| 79 | 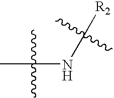 | 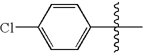 | 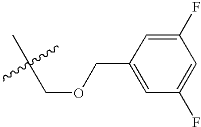 | [4-(4-Chloro-phenyl)- thiazol-2-yl]-(3,5- difluoro- benzyloxymethyl)- amine |

TABLE 2
Additional representative compounds of the invention.

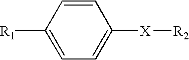

| # | X | R₁ | R₂ | Chemical name |
|---|---|---|---|---|
| 80 | 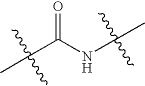 | 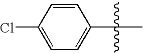 | 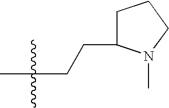 | 4′-Chloro-biphenyl-3-carboxylic acid[2-(1- methyl-pyrrolidin-2-yl)-ethyl]-amide |
| 81 | 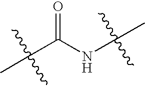 | 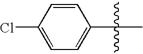 | 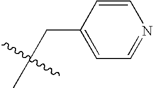 | 4′-Chloro-biphenyl-3-carboxylic acid (pyridin-4- ylmethyl)-amide |
| 82 | 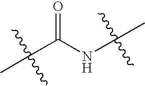 | 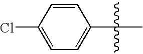 | 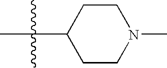 | 4′-Chloro-biphenyl-3-carboxylic acid (1-methyl- pipendin-4-yl)-amide |
| 83 | 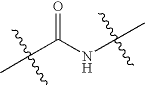 | 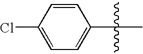 | 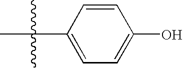 | 4′-Chloro-biphenyl-3-carboxylic acid (4- hydroxy-phenyl)-amide |
| 84 | 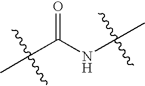 | 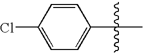 | 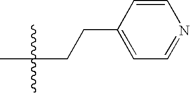 | 4′-Chloro-biphenyl-3-carboxylic acid (2-pyridin- 4-yl-ethyl)-amide |
| 85 | 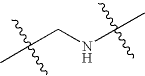 | 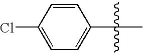 | 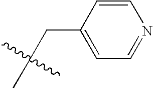 | (4′-Chloro-biphenyl-3-ylmethyl)-pyridin-4- ylmethyl-amine |
| 86 | 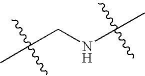 | 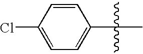 | 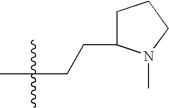 | (4′-Chloro-biphenyl-3-ylmethyl)-[2-(1-methyl- pyrrolidin-2-yl)-ethyl]-amine |

TABLE 3
Additional representative compounds of the invention.

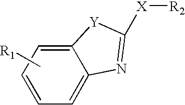

| # | X | Y | R₁ | R₂ | Chemical name |
|---|---|---|---|---|---|
| 87 | 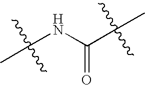 | O | 5-Chloro- | 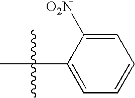 | N-(5-Chloro- benzooxazol-2-y)-2- nitro-benzamide |
| 88 | 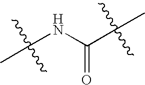 | O | 5-Chloro- | 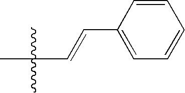 | N-(5-Chloro- benzooxazol-2-yl)-3- phenyl-acrylamide |
| 89 | 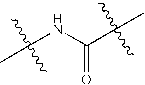 | O | 5-Chloro- | 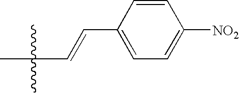 | N-(5-Chloro- benzooxazol-2-yl)-3-(4- nitro-phenyl)-acrylamide |
| 90 | 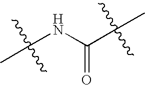 | O | 5-Chloro- | 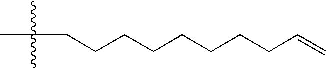 | Undec-10-enoic acid (5- chloro-benzooxazol-2-yl)- amide |
| 91 | 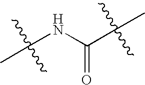 | O | 5-Chloro- | 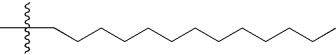 | Tetradecanoic acid (5- chloro-benzooxazol-2-yl)- amide. |
| 92 | 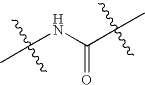 | O | 5-Chloro- | 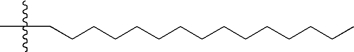 | Hexadecanoic acid (5- chloro-benzooxazol-2-yl)- amide |
| 93 | 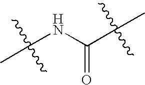 | O | 5-Chloro- | 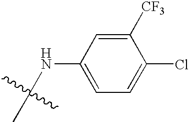 | 1-(5-Chloro-benzooxazol- 2-yl)-3-(4-chloro-3- trifluoromethyl-phenyl)- urea |
| 94 | 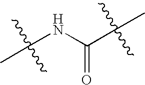 | S | H- | 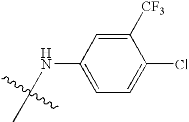 | 1-Benzothiazol-2-yl-3-(4- chloro-3-trifluoromethyl- phenyl)-urea |
| 95 | 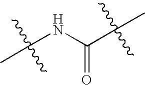 | S | 5-Chloro- | 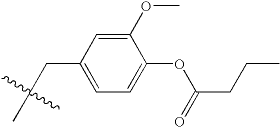 | Butyric acid 4-[(6-chloro- benzothiazol-2- ylcarbamoyl)-methyl]-2- methoxy-phenyl ester |
| 96 | 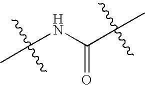 | S | H- | 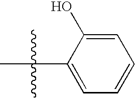 | N-(5-Chloro- benzothiazol-2-yl)-2- hydroxy-benzamide |
| 97 | 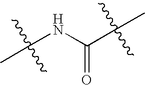 | O | 5-Chloro- | 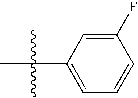 | N-(5-Chloro- benzooxazol-2-yl)-3- fluoro-benzamide |

General methods NMR spectra were obtained on Varian 300 instruments in CDCl₃. DMSO-d₆. Chemical shifts are quoted relative to TMS for ¹H- and 13 C-NMR spectra. Solvents were dried and distilled prior to use. Reactions requiring anhydrous conditions were conducted under an atmosphere of nitrogen and column chromatography was carried out over silica gel (Merck, silica gel 60, 230-400 mesh). All reagents and commercially available materials were used without further purification.

Example 1

### General Methods for the Synthesis of Compounds of this Invention

General approaches to the synthesis of compounds indicated in Tables 1, 2 and 3 are described in Scheme 1.

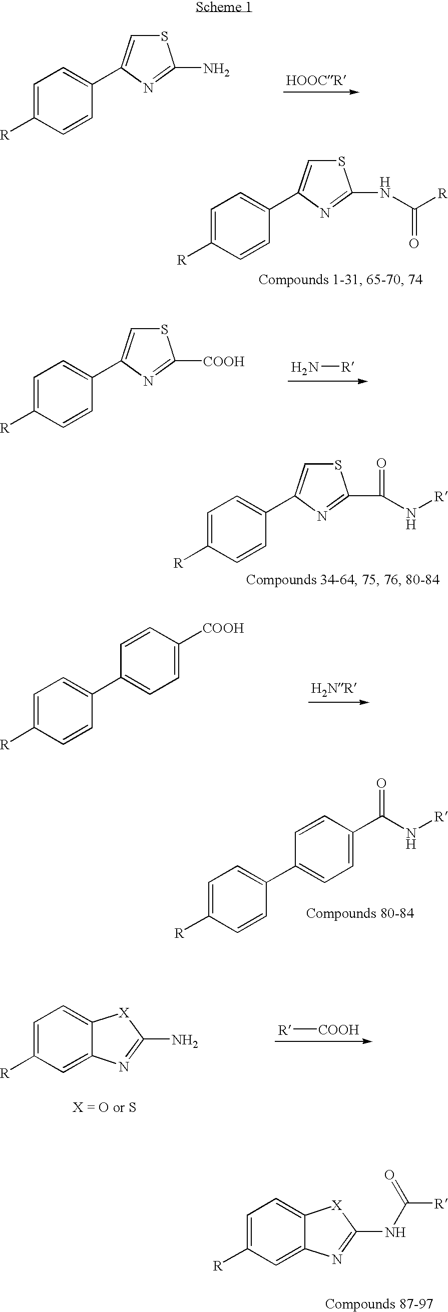

A diverse set of substituted compounds can be efficiently synthesized by condensation of various precursors with carboxylates or amines, and a wide variety of such compounds are commercially available. The following Examples provide several representatives of the products of this process; however, these methods can be adapted to produce many structurally related compounds that are considered to be subjects of this invention.

### Example 2

### Synthesis of Phenylthiazoles

The methods described below were used to prepare a library of substituted phenylthiazoles, biphenyls, benzooxazoles and benzothiazoles. Data provided below include: methods for synthesis, the amount synthesized, the yield of the amidation reaction, mass spectral (MS) data for the compound, and NMR spectral data for the compound.

### Compound 2. Tetradecanoic acid [4-(4-chloro-phenyl)-thiazol-2-yl]-amide (B231201)

Myristoyl chloride (47 mg, 0.19 mmol) was placed in a dried 100 mL round bottom reaction flask by syringe. Anhydrous dioxane (5 mL) was added to it, followed by addition of 2-amino-4-(4-chlorophenyl)thiazol (40 mg, 0.19 mmol) and pyridine (100 uL). The mixture was heated under reflux for 2 h. After cooling to RT and removal of the solvent the residue was dissolved in dichloromethane (50 mL) and washed with water (50 mL). The organic solution was dried over sodium sulfate. After filtration through a pad of silica eluted with chloroform and concentration, the residue that is not very soluble in chloroform was purified by chromatotron (silica, hexane-chloroform) to afford a white solid (73 mg, 0.17 mmol). R_{f}=0.38 (chloroform, silica). Y=91%, NMR and MS confirmed it is the target compound.

### Compound 3, Hexadecanoic acid [4-(4-chloro-phenyl)-thiazol-2-yl-amide (B231203)

Palmitoyl chloride (101 mg, 0.37 mmol) was placed in a dried 100 mL round bottom reaction flask by syringe. Anhydrous dioxane (8 mL) was added to it, followed by addition of 2-amino-4-(4-chlorophenyl)thiazol (79 mg, 0.37 mmol) and pyridine (100 uL). The mixture was heated under reflux for 2.5 h. After cooling to RT and removal of the solvent, the residue was dissolved in dichloromethane (50 mL) and washed with water (50 mL). The organic solution was dried over sodium sulfates After filtration through a pad of silica eluted with chloroform, the concentrated residue was purified by chromatotron (silica, hexane-chloroform) to afford a white solid (130 mg, 0.29 mmol). R_{f}=0.35 (chloroform, silica). Y=78%. NMR and MS confirmed it is the target compound.

### Compound 4. Undec-10-enoic acid [4-(4-chloro-phenyl)-thiazol-2-yl]-amide (B231205)

10-undecenoyl chloride (47 mg, 0.23 mmol) was placed in a dried 100 mL round bottom reaction flask by syringe Anhydrous dioxane (5 mL) was added to it, followed by addition of 2-amino-4-(4-chlorophenyl)thiazol (50 mg, 0.23 mmol) and pyridine (100 uL). The mixture was heated under reflux for 1.5 h. After cooling to RT and removal of the solvent, the residue was dissolved in dichloromethane (50 mL) and washed with water (50 mL). The organic solution was dried over sodium sulfate. After filtration through a pad of silica eluted with chloroform, the concentrated residue was purified by chromatotron (silica, chloroform) to afford a white solid (85 mg, 0.225 mmol). R_{f}=0.35 (chloroform, silica). Y=98%. NMR and MS confirmed it is the target compound

### Compound 5. N-[4-(4-Chloro-phenyl)-thiazol-2-yl]3-(4-nitro-phenyl)-acrylamide (B231209)

Trans-4-nitrocinnaoyl chloride (45 mg, 0.21 mmol) and 2-amino-4-(4-chlorophenyl)thiazol (45 mg, 0.21 mmol) were placed in a dried 100 mL round bottom reaction flask. Anhydrous dioxane (5 mL) was added to it, followed by addition of pyridine (100 uL). The mixture was heated under reflux for 1 h. After cooling to RT and removal of the solvent, the residue was dissolved in dichloromethane (50 mL) and washed with water (50 mL). The organic solution was dried over sodium sulfate. After filtration through a pad of silica eluted with 1% methanol in chloroform, the concentrated residue that was very difficult to be loaded on the chromatotron due to low solubility was purified by chromatotron (silica, chloroform) to afford a yellow solid (90 mg, 0.23 mmol). R_{f}=0.23 (1% methanol in chloroform, silica). Y>100%. NMR and MS confirmed it is the target compound.

### Compound 6. Octadec-9-enoic acid [4-(4-chloro-phenyl)-thiazol-2-yl]-amide (B231212)

Oleoyl chloride (45 mg, 0.15 mmol) was placed in a dried 100 mL round bottom reaction flask by syringe. Anhydrous dioxane (5 mL) was added to it, followed by addition of 2-amino-4-(4-chlorophenyl)thiazol (29 mg, 0.14 mmol) and pyridine (100 uL). The mixture was heated under reflux for 1.5 h. After cooling to RT and removal of the solvent, the residue was dissolved in dichloromethane (50 mL,) and washed with water (50 mL). The organic solution was dried over sodium sulfate. After filtration through a pad of silica eluted with chloroform, the concentrated residue was purified by chromatotron (silica, chloroform) to afford a white solid (30 mg, 0,063 mmol), R_{f}=0.41 (chloroform, silica). Y=45%. NMR and MS confirmed it is the target compound.

### Compound 7. N-[4-(4-Chloro-phenyl)-thiazol-2-yl]-3-phenyl-acrylamide (B231216)

Trans-cinnaoyl chloride (48 mg, 0.29 mmol) and 2-amino-4-(4-chlorophenyl)thiazol (61 mg, 0.29 mmol) were placed in a dried 100 mL round bottom reaction flask. Anhydrous dioxane (5 mL) was added to it, followed by addition of pyridine (100 uL). The mixture was heated under reflux for 1 h. After cooling to RT and removal of the solvent, the residue was dissolved in dichloromethane (50 mL) and washed with water (50 mL). The organic solution was dried over sodium sulfate. After filtration through a pad of silica eluted with 1% methanol in chloroform, the concentrated residue was purified by chromatotron (silica, chloroform) to afford a yellow solid (80 mg, 0.23 mmol). R_{f}=0.23 (chloroform, silica). Y=81%, NMR and MS confirmed it is the target compound.

### Compound 8. Butyric acid 4-{2-[4-(4-chloro-phenyl)-thiazol-2-ylcarbamoyl]-vinyl}-2-methoxy-phenyl ester (B240219)

### Butyric acid 4-(2-carboxy-vinyl)-2-methoxy-phenyl ester (B250427)

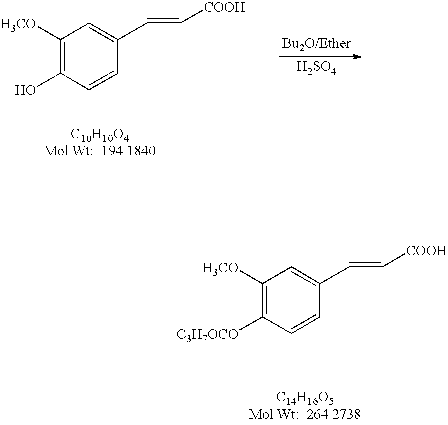

4-Hydroxy3-methoxycinnamic acid (100 g, 51.5 mmol) was mixed with Bu₂O (35 mL) to form a suspension, followed by addition of H₂SO₄ (0.8 mL). After stirring for 5 min, it became a yellow solution. Ether (200 mL) was added to it. The reaction mixture became an emulsion. The reaction was continued for overnight at RT (18 hours). The mixture was poured into 500 mL of ice-water. The mixture was extracted with EtOAc (300+200 mL). The EtOAc solution was dried over Na₂SO₄. After filtration and removal of the solvent, the oily liquid stood in the hood overnight. The solid appeared. After filtration, the solid was washed with plenty of hexane to afford a white solid (12.1 g, Y=89%). R_{f}=0.27 (5% MeOH in chloroform); ¹H NMR (CDCl₃) δ 7.75 (d, J=15.8 Hz, 1 H), 7.00-7.20 (m, 3 H), 6.40 (d, J=15.8 Hz, 1 H), 3.87 (s, 3 H, 2.58 (t, J=7.2 Hz, 2 H), 1.80 (dd, J=7.2 Hz, J=7.2 Hz, 2 H), 1.06 (t, J=7.2 Hz); ¹³C NMR (CDCl₃) δ 171.2, 171.0, 151.0, 144.4, 127.7, 123.3, 122.9, 113.7, 56.1, 35.9, 18.6, 13.7.

Butyric acid 4-(2-carboxy-vinyl)-2-methoxy-phenyl ester (1.078 g, 4.08 mmol) was suspended in dichloromethane (12 mL), followed by addition of 2 M oxalyl chloride in dichloromethane (3 mL) and DMF (150 uL). After 30 min stirring, the volatile components were removed in vacuo. The white residue was suspended in 1,4-dioxane (20 mL), followed by addition of 4-(4-Chloro-phenyl)-thiazol-2-ylamine (861 mg, 4.08 mmol) and pyridine (500 uL). It became a yellow suspension. The stirring of the mixture was continued at boiling for min. After cooling down to RT, the solvent was removed in vacuo. The residue was partitioned in water (50 mL) and ethyl acetate (100 mL). The organic phase was further washed with 0.5 N HCl (50 mL), 5% NaHCO₃ (50 mL) and water (50 mL). The organic solution was dried over sodium sulfate. After filtration through a pad of Celite and washed with plenty of ethyl acetate, the solvent was removed. The residue was partitioned in ethyl acetate (30 mL) and 5% NaHCO₃ (20 ml) and water (2×20 mL), and then dried over sodium sulfate. After filtration, the solvent was evaporated in hood. The residue was washed with plenty ethanol to afford a pure yellow compound (1.01 g, 2.21 mmol), Y=54% ¹H NMR (CDCl₃) δ 11.1 (br s, 1 H), 7.73 (d, J=8.7 Hz, 2 H), 7.64 (d, J=15.6 Hz, 1 H), 7.29 (d, J=8.7 Hz, 2 H), 7.20 (s, 1 H), 6.97 (d, J=7.8 Hz, 1 H), 6.85 (d, J=1.2 Hz, 1 H), 6.76 (dd, J=7.8 Hz, 1.2 Hz, 1 H), 6.14 (d,J=15.3 Hz, 1 H), 3.80 (s, 3 H), 2.59 (t, J=7.2 Hz, 2 H), 1,82 (dd, J=7.2 Hz, 7.2 Hz, 2 H), 1.07 (t, J=7.2 Hz, 3 H); ¹³C NMR (DMSO-d₆) δ170.7, 163.2, 157.9, 150.9, 147.17, 141.7, 140.7, 133.1, 133.0, 132.1, 128.6 (2C), 127.2 (2C), 123.3, 120.2, 119.6, 112.2, 109.1, 55.8, 35.0, 18.0, 13.3; MS (MALDI) m/z calcd for C₂₃H₂₂ClN₂O₄S (M+H⁺) 457, found 457.

### Compound 9. N-[4-(3-Chloro-phenyl)-thiazol-2-yl]-3-(4-hydroxy-3-methoxy-phenyl)-acrylamide (B240301)

4-Hydroxy3-methoxycinnamic acid (194 mg, 1 mmol) was dissolved in THF (7 mL) with the protection of Ar. The solution was chilled to 0° C. in ice-water bath, followed by addition of DCC (dicyclohexylcarbodiimide) solution [209 mg, 1.01 mmol in THE (5 mL) The mixture was stirred at 0° C. for a few minutes, followed by addition of HOBt (1-hydroxybenzotriazole) (135 mg, 1 mmol), 2-amino-5-chlorobenzothiazole (210 mg, 1 mmol) and DMAP (25 mg, 0.20 mmol). The mixture was stirred at 0° C. for 1 hour, then at RT for 24 hours. The solid appeared after reaction at RT for some time. After filtration through a pad of Celite washed with THF, the solvent was removed in vacuo. The residue was most dissolved in EtOAc (40 mL). There was some solid left on the glass wall of the flask. The EtOAc solution was washed with 10% citric acid (40 mL), sat NaHCO₃ (40 mL) and sat NaCl (40 mL). The organic solution was then dried over Na₂SO₄. After filtering through a pad of silica eluted with 5% MeOH in CHCl₃, the concentrated residue was purified by chromatotron (silica) eluted with CHCl₃ to afford a compound which was confirmed by ¹H NMR.

### Compound 10. Acetic acid 4-(2-[4-(4-chloro-phenyl)-thiazol-2-ylcarbamoyl]-vinyl -2-methoxy-phenyl ester (B240419)

4-Acetoxy3-methoxycinnamic acid (130 mg, 0.55 mmol) was suspended in dichloromethane (5 mL) with protection of Ar. 2 M oxalyl chloride in CH₂Cl₂ (0.7 mL) and DMF (50 μL) were added to it at RT. The suspension became a yellowish solution after 0.5 hour stirring. During this period, a lot of gas released. The volatile components were removed in vacuo and the yellowish residue was used directly for the next reaction. Dioxane (20 mL) was added to the crude acid chloride with the protection of Ar, followed by addition of 2-Amino-4-(4-chlorobenzo)thiazole (177 mg, 0.84 mmol) and pyridine (200 uL). The yellowish solution was heated to 100° C. (oil bath) for 2 hours. After cooling and removing the solvent under the reduced pressure, the residue was dissolved in EtOAc (50 mL) and washed with water (2×50 mL). The organic solution was dried over Na₂SO₄. After filtration and concentration, the residue was purified by Al₂O₃-n chromatography eluted with chloroform to afford the compound (14 mg). The structure of the compound was confirmed by NMR and MS,

### Compound 11. Butyric acid 2-butyryloxy-5-{2-[4-(4-chloro-phenyl)-thiazol-2-ylcarbamoyl]-vinyl}-phenyl ester (B240511)

### Butyric acid 2-butyryloxy-4-(2-carboxy-vinyl)-phenyl ester (B240427)

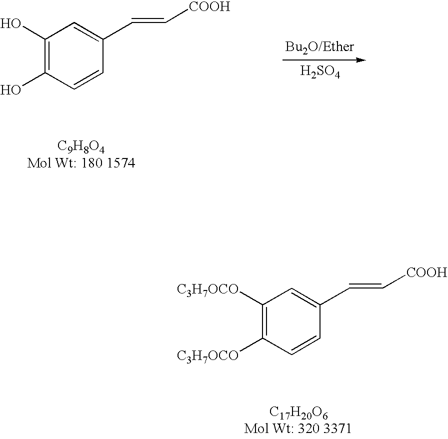

3,4-Dihydroxycinnamic acid (1.006 g, 5.6 mmol) was mixed with Butyric acid anhydride (7 mL), followed by addition of H₂SO₄ (0.1 mL). The mixture was stirred for 5 min. It became a dark solution. Ether (20 mL,) was added to it. The reaction was continued for 24 hours. The mixture was poured into 100 mL of ice-water. The water mixture was extracted with EtOAc (50 mL). The EtOAc solution was washed with water (50 mL). The washing makes the solution become an emulsion and very slowly divide into two phases. After separation two phases, the brown organic solution was dried over Na₂SO₄. After filtering and concentration, the residue was loaded on a silica column and eluted with CHCl₃-1% MeOW in CHCl3-3% MeOH in CHCl₃ to afford a yellowish solid (1.4 g, Y=78%).

3,4-dibutanoylcinnamic acid (326 mg, 1.02 mmol) was dissolved in dichloromethane (5 mL) under the protection of Ar. 2 M Oxalyl chloride in CH₂Cl₂ (1.8 mL) and DMF (50 μL) were added to it at RT. The suspension became a yellowish solution after 0.5 hour stirring. During this period, a lot of gas released. The volatile components were removed ii? vacuo and the yellowish residue was used directly for the next reaction. Dioxane (15 mL) was added to the crude acid chloride under the protection of Ar, followed by addition of 2-Amino-4-(4-chlorobenzo)thiazole (321 mg, 1.52 mmol) and pyridine (200 uL). The yellowish solution heating at 100° C. (oil bath) for 2 hours became a dark solution. After cooling and removing the solvent under the reduced pressure. The residue was dissolved in EtOAc (50 mL) and washed with water (2×50 mL). The organic solution was dried over Na₂SO₄. After concentration, the residue was purified by chromatography (CHCl₃). The crude compound obtained was further purified by precipitation in CHCl₃. The concentrated mother liquid was purified again by chromatotron (Silica, CHCl₃) to afford the compound (200 mg, Y=38%) that was confirmed by NMR and MS.

### Compound 12. Acetic acid 4-{2-[4-(4-chloro-phenyl)-thiazol-2-ylcarbamoyl]-vinyl}-phenyl ester (B240505)

### 3-(4-Acetoxy-phenyl)-acrylic acid (B240428)

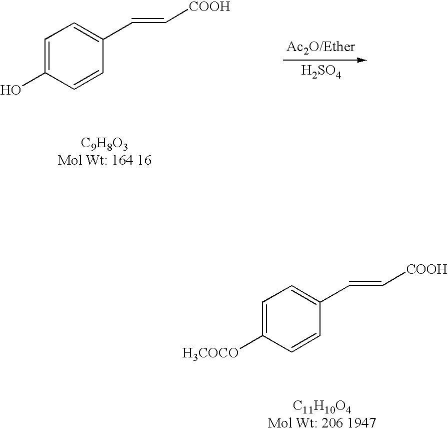

4-Hydroxycinnamic acid (1.002 g, 6.1 mmol) was mixed with Ac₂O (4 mL), followed by addition of H₂SO₄ (0.1 mL). It was stirred for 5 min and the mixture became a clear solution. Ether (20 mL) was added to it. The reaction was continued for 3 hours at RT. The solution was poured into 100 mL, of ice-water. The mixture was extracted with EtOAc (50 mL). The EtOAc solution was washed with water (50 mL) and dried over Na₂SO₄. After filtration, the concentrated residue was purified by the chromatography (3% MeOH in CHCl₃) to afford a white solid (280 mg, Y=22%). The structure of the compound was confirmed by NMR.

4-Acetoxylcinnamic acid (83 mg, 0.40 mmol) was suspended in dichloromethane (5 mL) under the protection of Ar. 2 M Oxalyl chloride in CH₂Cl₂ (0.7 mL) and DMF (50 μL) were added to it at RT. The suspension became a yellowish solution after 0.5 hour stirring. During this period, a lot of gas released. The volatile components were removed in vacuo and the yellowish residue was used directly for the next reaction. The crude acid chloride was suspended in pyridine (10 mL) under Are 2-Amino-4-(4-chlorophenyl)thiazole (114 mg, 0.54 mmol) was added to it in two portions. The mixture was heated at 100° C. for 0.5 hour. After cooling down to RT, the solvent was removed in vacuo. The residue was dissolved in EtOAc (50 mL) and washed with 5% NaHCO₃ (50 mL), IN HCl (50 mL) and water (50 mL). The EtOAc solution was dried over Na₂SO₄. After filtration, the concentrated residue was purified by chromatotron eluted with CHCl₃-1% MeOH in CHCl₃-3% MeOH in CHCl₃ to afford the compound. The structure of the compound was confirmed by NMR and MS.

### Compound 13. Butyric acid 2-{2-[4-(4-chloro-phenyl)-thiazol-2-ylcarbamoyl]-vinyl}-phenyl ester (B240624)

### Butyric acid 2-(2-carboxy-vinyl)-phenyl ester (B240609)

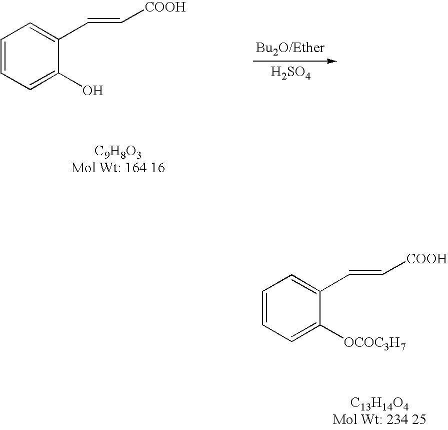

2-Hydroxycinnamic acid (1.0 g, 6.1 mmol) was mixed with Bu₂O (3.5 mL), followed by addition of H₂SO₄ (0.1 mL). After stirring for 5 min, the mixture became a yellow solution. Ether (20 mL) was added to it. The reaction was continued at RT overnight. The mixture was poured into 50 mL of ice-water and was extracted with EtOAc (50 mL). The EtOAc solution was washed with water (50 mL) and dried over Na₂SO₄. After filtration, the concentrated residue was purified by the chromatography (CHCl₃-3% MeOH in CHCl₃) to afford a white solid.

2-Butanoylcinnamic acid (128 mg, 0.55 mmol) was dissolved in dichloromethane (5 mL). 2 M Oxalyl chloride in CH₂Cl₂ (0.8 mL,) and DMF (50 μL) were added to it at RT. It became a yellowish solution. After 0.5 h stirring, the volatile components were removed in vacuo and the yellowish residue was used directly for the next reaction. Dioxane (20 mL) was added to the crude acid chloride under the protection of Ar, followed by addition of 2-amino-4-(4-chlorobenzo)thiazole (174 mg, 0.82 mmol). It became a suspension. After addition of pyridine (200 uL), the mixture became a solution again. The yellow solution was heated at 100° C. (oil bath) for 50 min. The reaction mixture became a dark green solution. After cooling and removing the solvent under the reduced pressure, the residue was dissolved in EtOAc (50 mL) and washed with water (2×50 mL). The organic solution was dried over Na₂SO₄. After filtration and concentration, the mixture was purified by chromatography eluted with chloroform to afford the impure compound that was further purified by chromatotron (silica) eluted with chloroform to afford the pure compound (9 mg) confirmed by MS.

### Compound 14

### Butyric acid 3-{2-[4-(4-chloro-phenyl)-thiazol-2-ylcarbamoyl]-vinyl}-phenyl ester (B240622)

### Butyric acid 3-(2-carboxy-vinyl)-phenyl ester (B240610)

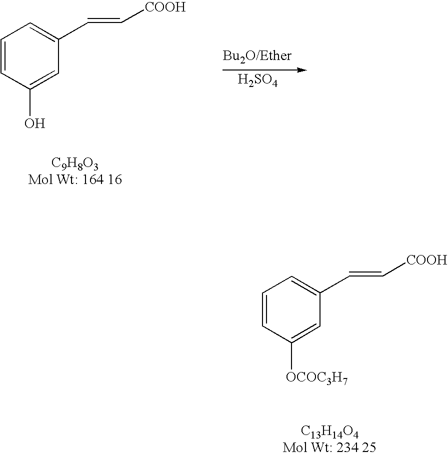

3-Hydroxycinnamic acid (1018 g, 6.2 mmol) was mixed with butyric acid anhydride (3.5 mL), followed by addition of H₂SO₄ (0.1 mL). After stirring for 5 min, the mixture became a brown solution. Ether (20 mL) was added to it. The reaction was continued at RT for 30 hours. The mixture was poured into 50 ml of ice-water and was extracted with EtOAc (50 mL,). Note, It was formed an emulsion and separation was very difficult. The EtOAc solution was washed with water (50 mL) and dried over Na₂SO₄. After filtration, the concentrated residue was purified by chromatography (CHCl₃-3% MeOH in CHCl₃, Si) to afford the white solid product (298 mg).

3-Butanoylcinnamic acid (50 mg, 0.21 mmol) was dissolved in dichloromethane (3 mL). 2 M oxalyl chloride in CH₂Cl₂ (0.3 mL) and DMF (50 μL) were added to it at RT. The solution became a yellowish solution. After 0.5 h reaction, the volatile components were removed in vacuo and the yellowish residue was used directly for the next reaction. Dioxane (10 mL) was added to the crude acid chloride under the protection of nitrogen, followed by addition of 2-amino-4-(4-chlorobenzo)thiazole (67 mg, 0.32 mmol) and pyridine (100 uL). The yellow solution was heated to 100° C. (oil bath) for 1 hour. The reaction mixture became a dark solution. After cooling and removing the solvent under the reduced pressure, the residue was dissolved in EtOAc (50 mL) and washed with water (2×50 mL). The organic solution was dried over Na₂SO₄. After filtration and concentration, the mixture was purified by chromatography eluted with chloroform to afford an impure compound that was further purified by chromatotron (silica) eluted with CHCl₃/hexane (4/1) to afford the pure product that was confirmed by MS,

### Compound 15. Butyric acid 4-(2-[4-(4-chloro-phenyl)thiazol-2-ylcarbamoyl]-vinyl}-phenyl ester (B240616)

### Butyric acid 4-(2-carboxy-vinyl)-phenyl ester (B240611)

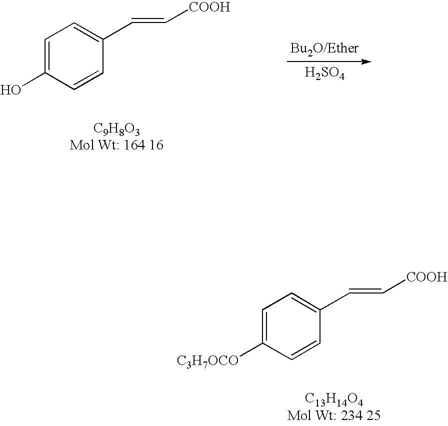

4-Hydroxycinnamic acid (1.029 g, 6.3 mmol) was mixed with butyric acid anhydride (3.5 mL), followed by addition of H₂SO₄ (0.1 mL). After stirring for 5 min, ether (20 mL) was added to this white suspension. The reaction mixture became a solution. The reaction was continued at RT for 24 hours. The mixture was poured into 50 mL of ice-water. The mixture was extracted with EtOAc (50 mL). The EtOAc solution was washed with water (50 mL) and dried over Na₂SO₄. After filtration, the concentrated residue was purified by the chromatography (CHCl₃-3% MeOH in CHCl₃) to afford a white solid.

4-Butanoylcinnamic acid (233 mg, 1.0 mmol) was dissolved in dichloromethane (5 mL). 2 M oxalyl chloride in CH₂Cl₂ (1.0 mL) and DMF (50 μL) were added to it at RT. After 0.5 hour reaction, the volatile components were removed in vacuo and the yellowish residue was used directly for the next reaction. Dioxane (10 mL) was added to the crude acid chloride under the protection of nitrogen, followed by addition of 2-amino-4-(4-chlorobenzo)thiazole (298 mg, 1.41 mmol) and pyridine (300 uL). The yellow solution was heated at 100° C. (oil bath) for 1 hour. The reaction mixture became a dark solution. After cooling and removing the solvent under the reduced pressure, the residue was dissolved in EtOAc (50 mL) and washed with water (2×50 mL). The organic solution was dried over Na₂SO₄. After filtration and concentration, the mixture was purified by chromatography eluted with chloroform to afford a crude product which was further purified by chromatotron (silica) eluted with CHCl₃ to afford a compound still with a little impurity. It was partially dissolved in CHCl₃. After filtration, the solid was collected to afford a pure white solid compound (170 mg, Y=40%) that was confirmed by MS.

### Compound 16. Butyric acid 4-{[4-(4-chloro-phenyl)-thiazol-2-ylcarbamoyl]-methyl}-2-methoxy-phenyl ester (B240816)

### Butyric acid 4-carboxymethyl-2-methoxy-phenyl ester (B240809)

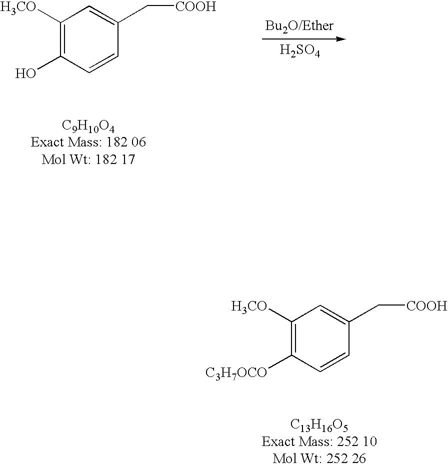

4-Hydroxy3-methoxyphenylacetic acid (974 mg, 5.3 mmol) was mixed with butyric acid anhydride (.3.5 mL), followed by addition of H₂SO₄ (0.1 mL). After stirring for 5 min, it became a yellow solution. Ether (20 mL) was added to it. The reaction was continued for 24 hours at RT. The mixture was poured into 50 mL of ice-water. The mixture was extracted with EtOAc (2×50 mL). The EtOAc solution was dried over Na₂SO₄. After filtration, the concentrated oily residue was purified by chromatography (CHCl₃-3% MeOHl in CHCl₃) to afford a yellowish white solid (961 mg, Y=72%). The structure of tie compound was confirmed by NMR.

4-Butanoyl3-methoxyphenylacetic acid (259 mg, 1.03 mmol) was dissolved in dichloromethane (5 mL). DMF (50 μL) and 2 M oxalyl chloride in CH₂Cl₂ (1.2 mL) were added to it at RT. After 0.5 hour stirring, the solvent was removed in in vacuo. The residue was dissolved in dioxane (10 mL). 2-Amino-4-(4-chlorophenyl)thiazole (309 mg, 1.46 mmol) was added to it, followed by addition of pyridine (300 uL). The mixture was heated at 100° C. for 1 hour. It became darker. After cooling down to RT, the solvent was removed in vacuo. The residue was dissolved in EtOAc (50 mL) and washed with water (50 mL+30 mL.). The combined water was extracted with EtOAc (30 mL). The EtOAc solution was dried over sodium sulfate After filtration and concentration, the residue was purified by chromatography eluted with CHCl₃. The collected fractions were concentrated and purified again by chromatotron (silica) eluted with hexane-CHCl₃ to afford a solid product (180 mg, Y=39%)

¹H NMR (CDCl₃) δ 9,67 (bs, 1 H), 7.72 (d, J=8.4 Hz, 2 H), 7.36 (d, J=8.4 Hz, 2 H), 7.13 (s, 1 H), 7.01 (d, J=8.1 Hz, 1 H), 6.83 (s, 1 H), 6.76 (d, J=8.1 Hz, 1 H), 3.80 (s, 3 H), 3.66 (s, 2 H), 2.57 (t, d=72 Hz, 2 H), 1.70-1.90 (m, 2 H, 1.05 (t, d=7.2 Hz, 3 H); ¹³C (CDCl₃) δ 171.6, 168.4, 151.5, 148,6, 139.5, 133.8, 132.6, 131.3, 128.8 (2 C), 127.2 (2 C), 123.5, 121 6, 113 4, 108.2, 55.9, 43.2, 35.9, 18.6, 13.7; MS (MALDI-TOF) m/z calcd for C₂₂H₂₂ ClN₂O₄S (M+H⁺) 445, found 445.

### Compound 17. Butyric acid 2-butyryloxy-5-{[4-(4-chloro-phenyl)-thiazol-2-ylcarbamoyl]-methyl}-phenyl ester (B240819)

### Butyric acid 2-butyryloxy-5-carboxymethyl-phenyl ester (B240810)

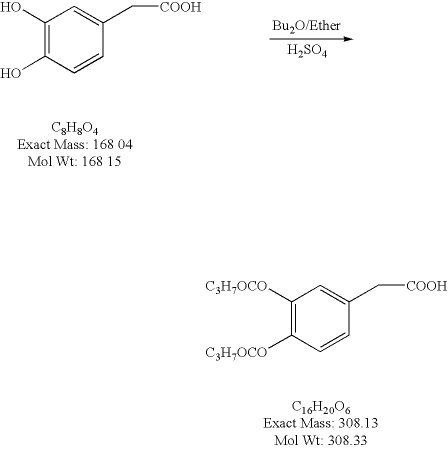

3,4-Dihydroxyphenylacetic acid (1.0 g, 6.0 mmol) was mixed with butyric acid anhydride (6 mL), followed by addition of H₂SO₄ (0.1 mL). After stirring for 5 min, it became a dark gray solution. Ether (20 mL) was added to it. The reaction was continued for 24 hours at RT. The mixture was poured into 50 mL of ice-water. It was extracted with EtOAc (2×50 mL). The EtOAc solution was dried over Na₂SO₄. After filtration and concentration, the oily residue was purified by chromatography (CHCl₃-3% MeOH in CHCl₃, Si) to afford a redish syrup (1.45 g, Y=78%). The structure of the compound was confirmed by NMR.

3,4-Dibutanoyl-phenylacetic acid (319 mg, 1.04 mmol) was dissolved in dichloromethane (5 mL). DMF (50 μL) and 2 M oxalyl chloride in CH₂Cl₂ (1.2 mL) were added to it at RT. After 0.5 hour stirring, the solvent was removed in vacuo. The residue was dissolved in dioxane (10 mL). 2-Amino-4-(4-chlorophenyl)thiazole (309 mg, 1.46 mmol) was added to it, followed by addition of pyridine (300 uL). The mixture was heated at 100° C. for 1 hour. After cooling down to RT, the solvent was removed in vacuo. The residue was dissolved in EtOAc (50 mL) and washed with water (50 mL+30 mL). The combined water was extracted with EtOAc (30 mL). The EtOAc solution was dried over sodium sulfate. After filtration and concentration, the residue was purified by chromatography eluted with CHCl₃. The collected fractions were concentrated and purified again by chromatotron (silica) eluted with hexane-CHCl-₃-1% MeOlH in chloroforn to afford a solid product (100 mg, Y=19%).

¹NMR (CDCl₃) δ 10.38 (bs, 1 H), 7.74 (d, J=8.4 Hz, 2 H), 7.37 (d, J=8.4 Hz, 2 H), 7.13 (s, 1 H), 7.10 (s, 1 H), 6.92-7.02 (m, 2 H), 3.49 (d, J=3.9 Hz, 2 H), 2.52 (t, J=7.4 Hz, 2 H), 2.51 (t, J=7.4 Hz, 2 H), 1.66-1.84 (m, 4 H), 0.96-1.14 (m, 6 H); ¹³C NMR (CDCl₃) δ170.9, 170.8, 168.1 (2 C), 148.4, 142.1, 141.5, 133.9, 132.6, 131.4, 128.9 (2 C), 127.4, 127.3 (2 C), 124.5, 123.8, 108.3, 41.9, 35.9 (2 C), 18.5, 18.4, 13.8, 13.7; MS (MALDI-TOF) m/z calcd for C₂₅H₂₅ClN₂O₅S (M+H⁺) 501, found 501.

### Compound 18. Butyric acid 5-{2-[4-(4-chloro-phenyl)-thiazol-2-ylcarbamoyl]-vinyl}-2-methoxy-phenyl ester (B240812)

3-Butanoyl-4-methoxycinnamic acid (277 mg, 1.05 mmol) was dissolved in dichloromethane (5 mL). DMF (50 μL) and 2 M oxalyl chloride in CH₂Cl₂ (1.2 mL,) were added to it at RT. After 0.5 hour stirring, the solvent was removed in vacuo. The residue was dissolved in dioxane (10 mL,) 2-Amino-4-(4-chlorophenyl)thiazole (306 mg, 1.45 mmol) was added to it, followed by addition of pyridine (300 uL). The mixture was heated at 100° C. for 0.5 hour. After cooling down to RT, the solvent was removed in vacuo. The residue was dissolved in EtOAc (50 mL) and washed with water (2×50 mL). The combined water was extracted with EtOAc (30 mL). The EtOAc solution was dried over sodium sulfate. After filtration and concentration, the residue was purified by chromatography eluted with hexane-CHCl₃. The collected fractions were concentrated and purified again by chromatotron (silica) eluted with hexane-CHCl₃ to afford a solid product (15 mg). NMR confirmed it was the compound.

### Compound 19. Butyric acid 2-methoxy-4-[2-(4-p-tolyl-thiazol-2-ylcarbamoyl)-vinyl]-phenyl ester (B240826)

### Butyric acid 5-(2-carboxy-vinyl)-2-methoxy-phenyl ester (B240803)

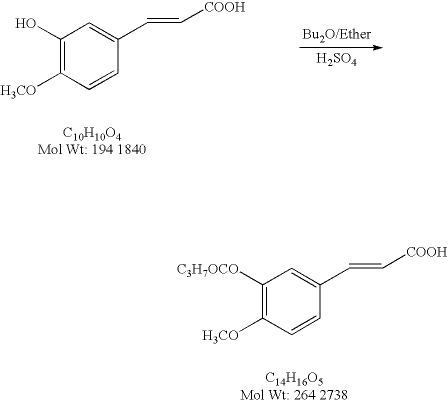

3-Hydroxy-4-methoxycinnamic acid (1.0 g, 5.15 mmol) was mixed with butyric acid anhydride (3.5 mL) to form a suspension, followed by addition of H₂SO₄ (0.1 mL,). After stirring for 5 min, it became a purple solution. Ether (20 mL,) was added to it. The reaction mixture became an emulsion, and then a yellow solution. The reaction was continued for 24 hours at RT. The mixture was poured into 50 mL of ice-water. The mixture was extracted with EtOAc (50 mL+30 mL). The EtOAc solution was dried over Na₂SO₄. After filtration, the concentrated oily residue was purified by chromatography (CDCl₃-3% MeOH in CHCl₃, Si) to afford a white solid (116 g, Y=85%). The structure of the compound was confirmed by NMR.

4-Butanoyl3-methoxycinnamic acid (98 mg, 0.37 mmol) was suspended in dichloromethane (5 mL). DMF (50 μL) and 2 M oxalyl chloride in CH₂Cl₂ (0.6 mL) were added to it at RT. The suspension became a yellowish solution. After 0.5 hour stirring, the solvent was removed in vacuo. The residue was dissolved in dioxane (5 mL), 2-Amino-4(p-toly)thiazole (107 mg, 0.56 mmol) was added to it, followed by addition of pyridine (110 uL). The mixture was heated at 100° C. for one hour. The mixture became dark yellow After cooling down to RT, the solvent was removed in vacuo. The residue was dissolved in EtOAc (50 mL) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with hexane-CHCl₃-1% Methanol in CHCl₃ to afford a solid product (70 mg, Y=43%). NMR confirmed it was the compound.

### Compound 20. Butyric acid 4-{2-[4-(4-bromo-phenyl)-thiazol-2-ylcarbamoyl]-vinyl}-2-methoxy-phenyl ester (B240827)

4-Butanoyl3-methoxycinnamic acid (91 mg, 0.34 mmol) was suspended in dichloromethane (5 mL) DMF (50 μL) and 2 M oxalyl chloride in CH₂Cl₂ (0.6 mL) were added to it at RT. The suspension became a yellowish solution. After 0.5 hour stirring, the solvent was removed in vacuo. The residue was dissolved in dioxane (5 mL). 2-Amino-4-(4-bromophenyl)thiazole (133 mg, 0.52 mmol) was added to it, followed by addition of pyridine (100 uL). The mixture was heated at 100° C. for one hour. After cooling down to RT, the solvent was removed in vacuo. The residue was dissolved in EtOAc (50 mL) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford the solid product (50 mg, Y=29%). NMR confirmed it was the compound

### Compound 21. 3-Benzo[1,3]dioxol-5-yl-N-[4-(4-chloro-phenyl)-thiazol-2-yl]-acrylamide (B240830)

3,4-(Methylenedioxy)cinnamic acid (195 mg, 1.02 mmol) was suspended in dichloromethane (5 mL). DMF (50 μL) and 2 M oxalyl chloride in CHl₂Cl₂ (1.5 mL) were added to it at RT After 0.5 hour stirring, the solvent was removed in vacuo. The residue was dissolved in dioxane (10 mL). 2-Amino-4-(4-chlorophenyl)thiazole (299 mg, 1.42 mmol) was added to it, followed by addition of pyridine (300 uL). The mixture was heated at 100° C. for 0.5 h. After cooling down to RT, the solvent was removed in vacuo. The residue was dissolved in EtOAc (50 mL) and washed with water (2×50 mL). The EtOAc solution was dried over sodium sulfate. After filtration and concentration, the residue was purified by chromatography [alumina (n)] eluted with CHCl₃. The collected fractions were concentrated. The precipitate was filtered and collected. It was the product (˜90 mg). The filtrate was purified by chromatotron (silica) eluted with CHCl₃ to afford second batch of the product (20 mg), (Y=28%), NMR confirmed it was the compound.

### Compound 22

### 2-Benzo[1,3]dioxol-5-yl-N-[4-(4-chloro-phenyl)-thiazol-2-yl]-acetamide (B240831)

3,4-(Methylenedioxy)phenylacetic acid (185 mg, 1.03 mmol) was dissolved in dichloromethane (5 mL) DMF (50 μL) and 2 M oxalyl chloride in CH₂Cl₂ (1.5 mL) were added to it at RT. After 0.5 hour stirring, the solvent was removed in vacuo. The residue was dissolved in dioxane (10 mL). 2-Amino-4-(4-chlorophenyl)thiazole (299 mg, 1.42 mmol) was added to it, followed by addition of pyridine (300 uL). The mixture was heated at 100° C. for 50 min. After cooling down to RT, the solvent was removed in vacuo. The residue was dissolved in EtOAc (50 mL) and washed with water (2×50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford a solid product (100 mg, Y=26%). NMR confirmed it was the compound.

### Compound 23. N-[4-(4-Chloro-phenyl)-thiazol-2-yl]3-(3,4-dimethoxy-phenyl)-propionamide (B240901)

3-(3,4-Dimethoxyphenyl)propionic acid (211 mg, 1.0 mmol) was dissolved in dichloromethane (5 mL). DMF (50 μL) and 2 M oxalyl chloride in CH₂Cl₂ (1.5 mL) were added to it at RT. After 0.5 hour stirring, the solvent was removed in vacuo. The residue was dissolved in dioxane (10 mL). 2-Amino-4-(4-chlorophenyl)thiazole (298 mg, 1.41 mmol) was added to it, followed by addition of pyridine (300 uL). The mixture was stirred at RT for 0.5 h and then at 100° C. for one hour. After cooling down to RT, the solvent was removed in vacuo. The residue was dissolved in EtOAc (50 mL) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford the solid product (200 mg, Y=50%). NMR and MS confirmed it was the target compound

### Compound 24. Butyric acid 4-[4-(4-chloro-phenyl)-thiazol-2-ylcarbamoyl]-2-methoxy-phenyl ester (B240902)

4-Butanoyl3-methoxybezoic acid (114 mg, 0.48 mmol) was dissolved in dichloromethane (5 mL). DMF (50 μL) and 2 M oxalyl chloride in CH₂Cl₂ (0.7 mL) were added to it at RT. After 0.5 hour stirring, the solvent was removed in vacuo. The residue was dissolved in dioxane (10 mL) 2-Amino-4-(4-chlorophenyl)thiazole (146 mg, 0.69 mmol) was added to it, followed by addition of pyridine (150 uL). The mixture was heated at 100° C. for 0.5 hour. After cooling down to RT, the solvent was removed in Vacuo. The residue was dissolved in EtOAc (50 mL,) and washed with water (50 mL). The EtOAc solution was dried over sodium sulfate After filtration and concentration, the residue was purified by chromatography eluted with CHCl₃ The collected fractions were concentrated and purified again by chromatotron (silica) eluted with hexane-CHCl₃ to afford a solid product (90 mg, Y==44%), NMR and MS confirmed it was the compound

### Compound 25. Butyric acid 2-butyryloxy-4-[4-(4-chloro-phenyl)-thiazol-2-ylcabamoyl]-phenyl ester (B240921)

### 3,4-Bis-butyryloxy-benzoic acid (B240907)

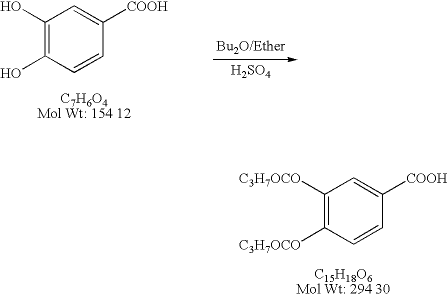

3,4-Dihydroxybenzoic acid (1,025 g, 6.7 mmol) was mixed with butyric acid anhydride (6.5 mL) to form a suspension, followed by addition of H₂SO₄ (0.1 mL). After stirring for 5 min, it became a solution Ether (20 mL) was added to it. The reaction was continued for 24 hours at RT. The mixture was poured into 50 mL of ice-water and was extracted with EtOAc (50 mL). The EtOAc solution was dried over Na₂SO₄. After filtration and concentration, the oily residue was purified by chromatography (CHCl₃-3% MeOH in CHCl₃) to afford the product (1.45 g, Y=74%),

3,4-Dibutyryloxybenzoic acid (216 mg, 0.73 mmol) was dissolved in dichloromethane (5 mL). DMF (50 μL) and 2 M oxalyl chloride in CH₂Cl₂ (0.6 mL) were added to it at RT The mixture became a yellowish solution. After 0.5 hour stirring, the solvent was removed in vacuo. The residue was dissolved in dioxane (10 mL). 2-Amino-4-(4-chlorophenyl)thiazole (210 mg, 1.0 mmol) was added to it, followed by addition of pyridine (150 uL). The mixture was heated at 100° C. for 1 hour. After cooling down to RT, the solvent was removed in vacuo. The residue was dissolved in EtOAc (50 mL) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford the product (120 mg, Y=34%).

¹H NMR (CDCl₃) δ 10.01 (bs, 1 H), 7.70-7.80 (m, 4 H), 7.35 (d, J=8.7 Hz, 2 H), 7.30 (dd, J=8.44 Hz, J=1.8 Hz, 1 H), 7.18 (s, 1 H), 2.55 (t, d=7.5 Hz, 2 H), 2.54 (t, d=7.5 Hz, 2 H), 1.70-1.86 (m, 4 H), 1.06 (t, d=7.5 Hz, 3 H), 1.05 (t, d=75 Hz, 3 H); ¹³C NMR (CDCl₃) δ 170.5, 170.2, 163.0, 158.2, 148.9, 145.7, 142.3, 133.7, 132.5, 130.0, 128.7 (2 C), 127.2 (2 C), 125.4, 123.9, 123.1, 108.5, 35.9, 35.8, 18.5, 18.4, 13.7 (2 C); MS (MALDI-TOF) m/z calcd for C₂₄H₂₄ClN₂O₅S (M+H⁺) 487, found 487.

### Compound 26. Butyric acid 2-butyryloxy-4-{2-[4-(4-chloro-phenyl)-thiazol-2-ylcarbamoyl]-ethyl}-phenyl ester (B240920)

### Butyric acid 2-butyryloxy-4-(2-carboxy-ethyl)-phenyl ester (B240908)

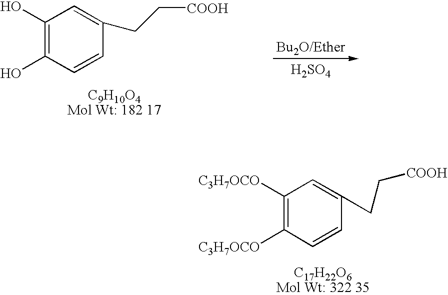

3,4-Dihydroxyhydrocinnamic acid (1.0 g, 5.5 mmol) was mixed with butyric acid anhydride (5.5 mL) to form a brown suspension, followed by addition of H₂SO₄ (0.1 mL). After stirring for 5 min, it became a brown solution. Ether (20 mL) was added to it. The reaction was continued at RT for 24 hours. The mixture was poured into 50 mL, of ice-water. The mixture was extracted with EtOAc (50 mL). The EtOAc solution was dried over Na₂SO₄. The concentrated oily residue was purified by chromatography (CHCl₃-3% MeOH in CHCl₃) to afford a semisolid (1.45 g, Y=82%).

3,4-Dibutanoylhydrocinnamic acid (138 mg, 0.43 mmol) was dissolved in dichloromethane (5 mL). DMF (50 μL) and 2 M oxalyl chloride in CH₂Cl₂ (0 6 mL) were added to it at RT. After 0.5 hour stirring, the solvent was removed in vacuo. The residue was dissolved in dioxane (10 mL,) 2-Amino-4-(4-chlorophenyl)thiazole (200 mg, 0.95 mmol) was added to it, followed by addition of pyridine (150 uL). The mixture was heated at 100° C. for 1 hour. After cooling down, the solvent was removed in vacuo. The residue was dissolved in EtOAc (50 mL,) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford the solid product (140 mg, Y=63%). ¹H NMR (CDCl₃) δ 11.21 (bs, 1 H), 7.65 (d, J=84 Hz, 2 H), 7.32 (d, J=8.4 Hz, 2 H), 7.10 (s, 1 H), 7.03 (d, J=8.7 Hz, 1 H), 6.74-6.80 (m, 2 H), 2.79 (t, J=8.0 Hz, 2 H), 2.52 (t J=7.5 Hz, 4 H), 2.20 (t J=8.0 Hz, 2 H), 1.70-1.84 (m, 4 H), 1.04 (t, J=7.5 Hz, 6 H); ¹³C NMR (CDCl₃) δ 171.0, 170.8, 169.9, 159.4, 148.2, 141.8, 140.4, 138.5, 133.9, 132.6, 129.0 (2 C), 127.4 (2 C), 126.0, 123.3, 123,0, 108.4, 37.0, 35.9 (2 C), 29.8, 18.5 (2 C), 13.7 (2 C); MS (MALDI-TOF) m/z calcd for C₂₆H₂₈ClN₂O₅S (M+H⁺) 515, found 515.

### Compound 27. Butyric acid 2,6-bis-butyryloxy-4-[4-(4-chloro-phenyl)-thiazol-2-ylcarbamoyl]-phenyl ester (B240914)

3,4,5-Tris-butyryloxy-benzoic acid (385 mg, 1.01 mmol) was dissolved in dichloromethane (10 mL). DMF (50 μL) and 2 M oxalyl chloride in CH₂Cl₂ (2 mL) were added to it at RT. The suspension became a yellowish solution. After 0.5 hour stirring, the solvent was removed in vacuo. The residue was dissolved in dioxane (10 mL). 2-Amino-4-(4-chlorophenyl)thiazole (301 mg, 1.43 mmol) was added to it, followed by addition of pyridine (200 uL). The mixture was heated at 100° C. for 0.5 hour. After cooling down to RT, the solvent was removed in vacuo. The residue was dissolved in EtOAc (50 mL) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford the solid product (87 mg, Y=15%).

¹H N (CDCl₃) δ 7.74 (d, J=8.4 Hz, 2 H), 7.69 (s, 1 H), 7.68 (s, 1 H), 7.36 (d, J=8.4 Hz, 2 H), 7.17 (s, 1 H), 2.46-2.58 (m, 6 H), 1.68-1.86 (m, 6 H), 1.05 (t, =7.2 Hz, 9 H); ¹³C NMR (CDCl₃) 170.1 (2 C), 168.9, 162.3, 157.8, 148.9, 143.8 (2 C), 138.3, 133,7, 132.6, 129.6, 128.8 (2 C), 127.2 (2 C), 120.0 (2 C), 108.5, 35.9 (2 C), 35.6, 18.5, 18.4 (2 C), 13.7 (3 C); MS (MALDI-TOF) m/z calcd for C₂₈H₂₉ClN₂O₇S (M+H⁺) 573, found 573.

### Compound 28. Butyric acid 4-{2-[4-(4-fluoro-phenyl)-thiazol-2-ylcarbamoyl]-vinyl}-2-methoxy-phenyl ester (B240927)

4-Butanoyl3-methoxycinnamic acid (40 mg, 0.15 mmol) was suspended in dichloromethane (5 mL). DMF (20 μL) and 2 M oxalyl chloride in CH₂Cl₂ (1 mL) were added to it at RT. The suspension became a yellowish solution After 0.5 hour stirring, the solvent was removed in vacuo. The residue was dissolved in dioxane (5 mL). 2-Amino-4-(4-fluorophenyl)thiazole (47 mg, 0.24 mmol) was added to it, followed by addition of pyridine (50 uL). The mixture was heated at 100° C. for 0.5 hour. After cooling down, the solvent was removed in vacuo. The residue was dissolved in EtOAc (50 mL) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford the solid product (30 mg, Y=45%). NMR and MS confirmed it was the target compound.

### Compound 29. Butyric acid 4-{2-[4-(4-chloro-phenyl)-thiazol-2-ylcarbamoyl]-ethyl)-2-methoxy-phenyl ester (B241116)

### Butyric acid 4-(2-carboxy-ethyl)-2-methoxy-phenyl ester (B241028)

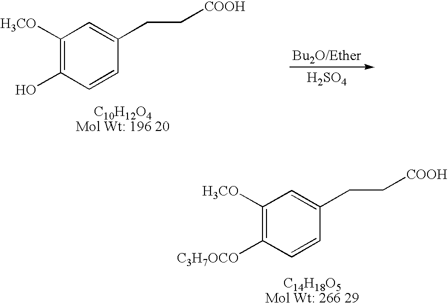

3-(4-Hydroxy3-methoxyphenyl)propionic acid (917 mg, 4.68 mmol) was mixed with butyric acid anhydride (3-5 mL) to form a suspension, followed by addition of H₂SO₄ (0.1 mL). It became a brown solution. After stirring for 5 min, ether (20 mL) was added to it. The color was turned to yellow. The reaction was continued for 24 hours at RT. The mixture was poured into 50 mL of ice-water and was extracted with EtOAc (50 mL). The EtOAc solution was dried over Na₂SO₄. After filtration and concentration, the oily residue was purified by chromatography eluted with hexane-chloroform-3% MeOH in chloroform to afford a yellowish solid (610 mg, Y=49%).

3,4-Dibutanoylhydrocinnamic acid (207 mg, 0.78 mmol) was dissolved in dichloromethane (10 mL). DMF (50 μL) and 2 M oxalyl chloride in CH₂Cl₂ (1 mL) were added to it at RT. After 0.5 hour stirring, the solvent was removed in vacuo. The residue was dissolved in dioxane (10 mL). 2-Amino-4-(4-chlorophenyl)thiazole (185 mg, 0.88 mmol) was added to it, followed by addition of pyridine (200 uL). The mixture was heated at 100° C. for 1 hour. After cooling down, the solvent was removed in vacuo. The residue was dissolved in EtOAc (50 mL,) and washed with water (50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford the solid product (210 mg, Y=47%). NMR confirmed it was the target compound.

### Compound 30. Butyric acid 4-{[4-(4-chloro-phenyl)-thiazol-2-ylcarbamoyl]-methyl}-2-nitro-phenyl ester (B241119)

### Butyric acid 4-carboxymethyl-2-nitro-phenyl ester (B241112)

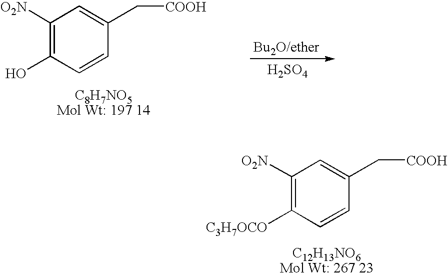

4-Hydroxy3-nitrophenylacetic acid (945 mg, 4.8 mmol) was mixed with butyric acid anhydride (3.5 mL), followed by addition of H₂SO₄ (0.1 mL). After stirring for 5 min, it became a yellow solution Ether (20 mL) was added to it. The reaction was continued at RT for 24 hours. The mixture was poured into 50 mL of ice-water and was extracted with EtOAc (2×50 mL) The EtOAc solution was dried over Na₂SO₄. After filtration and concentration, the oily residue was purified by chromatography (CHCl₃-3% MeOH in CHCl₃) to afford a yellowish white solid (1.01 g, Y=79%)

3,4-(Methylenedioxy)phenylacetic acid (223 mg, 0.84 mmol) was dissolved in dichloromethane (10 mL). DMF (50 μL) and 2 M oxalyl chloride in CH₂Cl₂ (1 mL) were added to it at RT. After 0.5 hour stirring, the solvent was removed in vacuo. The residue was dissolved in dioxane (10 mL). 2-Amino-4-(4-chlorophenyl)thiazole (201 mg, 0.95 mmol) was added to it followed by addition of pyridine (200 uL). The mixture was stirred at 100° C. for 50 min. The mixture became dark from yellow. After cooling down to RT, the solvent was removed in vacuo. The residue was dissolved in EtOAc (50 mL) and washed with water (2×50 mL). The EtOAc solution was dried over sodium sulfate. After filtration and concentration, the residue was purified by chromatography eluted with CHCl₃. The collected fractions were concentrated and purified again by chromatotron (silica) eluted with CHCl₃ to afford a solid product (62 mg, Y=16%). NMR confirmed it was the target compound.

### Compound 31. Butyric acid 2-amino-4-{[4-(4-chloro-phenyl)-thiazol-2-ylcarbamoyl]-methyl}-phenyl ester (B250106)

Butyric acid 4-{[4-(4-chloro-phenyl)-thiazol-2-ylcarbamoyl]-methyl }-2-nitrophenyl ester (17 mg, 0.04 mmol) and 10% Pd/C (3 mg) were suspended in ethanol (0.7 mL). The mixture was shaken under hydrogen pressure starting from 30 psi for 3 hours. The hydrogen pressure was decreased to about 20 psi. TLC was shown that there was no starting material left. There were two spots under UV. After filtration and removal of the solvent, the residue was purified by chromatography (alumina) eluted with 3% MeOH in chloroform to afford the compound (R_{f}=0.42, 5% MeOH in chloroform). The structure of the compound was confirmed by MS.

### Compound 32. 4-(4-Chloro-phenyl)-thiazole-2-carboxylic acid ethyl ester (B241228)

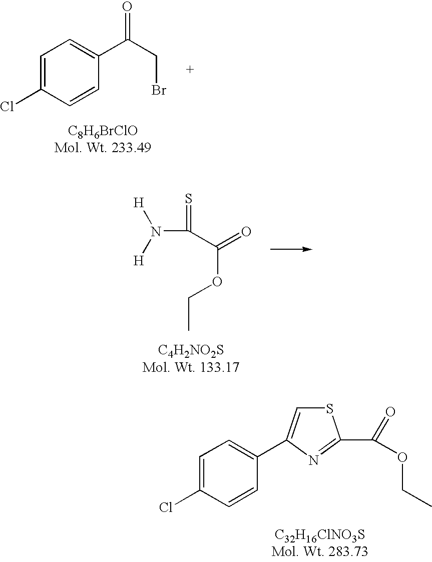

2-Bromo-4′-chloroacetophenone (7.0 g, 30 mmol) was dissolved in ethanol (200 mL). Ethyl thiooxamate (4.0 g, 30 mmol) and pyridine (2.5 mL) were added to it. The yellow solution was refluxed for 1 hour (oil bath). After cooling to room temperature, the solution was poured into ice-water (500 mL). After filtration, the precipitate was washed with cold water. The collected solid was dried to afford an off-white solid (4.7 g, Y=55%). The structure of the compound was confirmed by MNR.

### Compound 33. 4-(4-Chloro-phenyl)-thiazole-2-carboxylic acid hydrochloride salt (B241227)

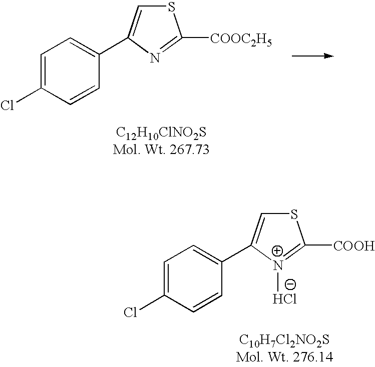

4-(4-Chloro-phenyl)-thiazole-2-carboxylic acid ethyl ester (268 mg, 1 mmol) was suspended in ethanol (10 mL), followed by addition of 1N NaOH (3 mL, 3 mmol). The suspension became a yellow solution. The mixture was heated for reflux for 0.5 hour. After cooling to RT, the volatile solvent was removed in vacuo. The mixture was diluted with water (20 mL) and washed with ether. The water solution was acidified by 1N MCl to PH≦2 and extracted with CH₂Cl₂ (2×20 mL,). The organic solution was dried over sodium sulfate. After filtration and removal of the solvent in vacuo, obtained a yellowish solid (245 mg, Y=89%).

### Compound 34. 4-(4-Chloro-phenyl)thiazole-2-carboxylic acid (pyridin-4-ylmethyl)amide (B241229)

4-(4-Chloro-phenyl)-thiazole-2-carboxylic acid (100 mg, 0.36 mmol) was dissolved in THF (5 mL), followed by addition of CDI (carbonyldiimidazole, 70 mg, 0.43 mmol). The slurry mixture was stirred at RT for 1 hour, 4-(Aminomethyl)pyridine (45 μL, 0.42 mmol) was added to it. The slurry mixture became a clear yellow solution. The reaction was continued at RT for 15 hours. After removal of the solvent, the residue was dissolved in dichloromethane (50 mL). It was washed with water (50 mL) and 5% sodium bicarbonate (50 mL). The organic solution was dried over sodium sulfate. After filtration through a pad of silica eluted with 5% MeOH in chloroform and concentration, the residue was applied on chromatotron (silica) eluted with chloroform-3% MeOH in chloroform to afford an off white solid (about 70 mg, Y=59%), R_{f}(5% MeOH in chloroform, Silica) was 0.29.

### Compound 35. 4-(4-Chloro-phenyl)-thiazole-2-carboxylic acid 4-dimethylamino-benzylamide (B241230)

4-(4-Chloro-phenyl)-thiazole-2-carboxylic acid (103 mg, 0.37 mmol) was dissolved in THF (5 mL), followed by addition of CDI (73 mg, 0.44 mmol). The slurry mixture was stirred at RT for 1 hour. 4-(Dimethylamino)benzylamine (65 mg, 0.43 mmol) was added to it. The slurry mixture became a clear yellow solution. The reaction was continued at RT for overnight. After removal of the solvent, the residue was dissolved in dichloromethane (50 mL). It was washed with water (50 mL) and 5% sodium bicarbonate (50 mL). The organic solution was dried over sodium sulfate. After filtration through a pad of silica eluted with 5% MeOH in chloroform and concentration, the residue was applied on chromatotron (silica) eluted with chloroform-3% MeOH in chloroform to afford a component with R_{f}═0.7 (5% MeOH in chloroform, Silica). It was a yellow solid (about 50 mg, Y=36%) NMR was shown that was the compound.

### Compound 36. 4-(4-Chloro-phenyl)-thiazole-2-carboxylic acid 3,5-difluoro-benzylamide (B250108)

4-(4-Chloro-phenyl)-thiazole-2-carboxylic acid (70 mg, 0.25 mmol) was dissolved in THE (5 mL), followed by addition of CDI (57 mg, 0.35 mmol) The slurry mixture was stirred at RT for 1 hour, 3,5-Difluorobenzylamine (44 mg, 0.31 mmol) dissolved in THF (2 mL) was added to it. The slurry mixture became a clear yellow solution. The reaction was continued at RT overnight. It became a suspension. After removal of the solvent, the residue was washed twice with water (2×10 mL). After filtration, the residue was dissolved in dichloromethane (40 mL,) and washed with 0.5 N HCl (20 mL) and water (20 mL). The organic solution was dried over sodium sulfate. After filtration through a pad of silica eluted with chloroform, the solvent was removed to afford a yellow solid (63 mg, Y=69%), R_{f} value (chloroform, Silica, UV) was 0.5.

### Compound 37. 4-(4-Chloro-phenyl)-thiazole-2-carboxylic acid 4-chloro3-trifluoromethyl-benzylamide (B250109)

4-(4-Chloro-phenyl)-thiazole-2-carboxylic acid (63 mg, 0.23 mmol) was dissolved in THF (5 mL,), followed by addition of CDI (50 mg, 0.31 mmol). The slurry mixture was stirred at RT for 1 hour, 4-Chloro3-(trifluoromethyl)benzylamine (55 mg, 0.26 mmol) dissolved in THF (2 mL) was added to it. The reaction was continued at RT overnight. It became a suspension. After removal of the solvent the residue was washed twice with water (2×10 mL). After filtration, the residue was dissolved in dichloromethane (40 mL) and washed with 0.5 N HCl (20 mL) and water (20 mL). The organic solution was dried over sodium sulfate. R_{f} value (chloroform, Silica, UV) was 0.55. After filtration through a pad of silica eluted with chloroform, the solvent was removed to afford a yellow solid (65 mg, Y=66%).

### Compound 38. 4-(4-Chloro-phenyl)-thiazole-2-carboxylic acid 2-chloro-4-fluoro-benzylamide (B250110)

4-(4-Chloro-phenyl)-thiazole-2-carboxylic acid (61 mg, 0.25 mmol) was dissolved in THF (5 mL), followed by addition of CDI (50 mg, 0.31 mmol). The slurry mixture was stirred at RT for 1 hour. 2-Chloro4-fluorobenzylamine (41 mg, 0.26 mmol) dissolved in THF (2 mL) was added to it. The slurry mixture became a clear yellow solution. The reaction was continued at RT overnight. After removal of the solvent, the residue was washed twice with water (2×10 mL). After filtration, the residue was dissolved in dichloromethane (40 mL) and washed with 0.5 N HCl (20 mL) and water (20 mL). The organic solution was dried over sodium sulfate. R_{f} value (chloroform, Silica) was 0.55. After filtration through a pad of silica eluted with chloroform, the solvent was removed to afford a semisolid (63 mg, Y=66%).

### Compound 39. 4-(4-Chloro-phenyl)-thiazole-2-carboxylic acid 3-chloro-4-fluoro-benzylamide (B250111)

4-(4-Chloro-phenyl)-thiazole-2-carboxylic acid (62 mg, 0.22 mmol) was dissolved in THF (5 mL), followed by addition of CDI (45 mg, 0.28 mmol). The slurry mixture was stirred at RT for 1 hour. 3-Chloro4-fluorobenzylamine (41 mg, 0.26 mmol) dissolved in THF (2 mL) was added to it. The reaction was continued at RT overnight. After removal of the solvent, the residue was washed twice with water (2×10 mL). After filtration, the residue was dissolved in dichloromethane (40 mL) and washed with 0.5 N HCl (20 mL) and water (20 mL). The organic solution was dried over sodium sulfate. R_{f} value (chloroform, Silica) was 0.32. After filtration through a pad of silica eluted with chloroform and concentration, the residue was applied on chromatotron (silica) eluted with chloroform to afford a solid (61 mg, Y=73%),

### Compound 40. 4-(4-Chloro-phenyl)-thiazole-2-carboxylic acid 3,4-difluoro-benzylamide (B250112)

4-(4-Chloro-phenyl)-thiazole-2-carboxylic acid (62 mg, 0.22 mmol) was dissolved in THE (5 mL), followed by addition of CDI (44 mg, 0.27 mmol). The slurry mixture was stirred at RT for 1 hour 3,4-Difluorobenzylamine (41 mg, 0.29 mmol) dissolved in THF (2 mL) was added to it. The slurry mixture became a clear yellow solution. The reaction was continued at RT overnight. After removal of the solvent, the residue was washed twice with water (2×10 mL). After filtration, the residue was dissolved in dichloromethane (40 mL,) and washed with 0.5 N HCl (20 mL) and water (20 mL). The organic solution was dried over sodium sulfate. R_{f} value (chloroform, Silica) was 0.28. After filtration through a pad of silica eluted with chloroform and concentration, the residue was applied on chromatotron (silica) eluted with chloroform to afford a solid (56 mg, Y=70%).

### Compound 41. 4-(4-Chloro-phenyl)-thiazole-2-carboxylic acid [2-(3-bromo-4-methoxy-phenyl)-ethyl}-amide (B250113)

4-(4-Chloro-phenyl)-thiazole-2-carboxylic acid (59 mg, 0.21 mmol) was dissolved in THE (5 mL), followed by addition of CDI (44 mg, 0.27 mmol). The slurry mixture was stirred at RT for 1 hour. 3-Bromo-4-methoxyphenethylamine (60 mg, 0.26 mmol) dissolved in THE (2 mL) was added to it. The reaction was continued at RT overnight. After removal of the solvent, the residue was washed twice with water (2×10 mL). After filtration, the residue was dissolved in dichloromethane (40 mL) and washed with 0.5 N HCl (20 mL) and water (20 mL). The organic solution was dried over sodium sulfate. R_{f} value (chloroform, Silica) was 0.22. After filtration through a pad of silica eluted with 1% MeOH in chloroform and concentration, the residue was applied on chromatotron (silica) eluted with chloroform to afford a solid (54 mg, Y=59%). NMR confirmed it is the compound.

### Compound 42. 4-(4-Chloro-phenyl)-thiazole-2-carboxylic acid 3,4,5-trifluoro-benzylamide (B250114)

4-(4-Chloro-phenyl)-thiazole-2-carboxylic acid (60 mg, 0.22 mmol) was dissolved in THE (5 mL), followed by addition of CDI (62 mg, 0.38 mmol). The slurry mixture was stirred at RT for 1 hour. 3-Bromo-4-methoxyphenethylamine (45 mg, 0.28 mmol) dissolved in THE (2 mL) was added to it. The reaction was continued at RT overnight. After removal of the solvent, the residue was washed twice with water (2×10 mL). After filtration, the residue was dissolved in dichloromethane (40 mL) and washed with 0.5 N HCl (20 mL), 5% NaHCO₃ (20 mL) and water (20 mL). The organic solution was dried over sodium sulfate. R_{f} value (chloroform, Silica) was 0.29. After filtration through a pad of silica eluted with chloroform, the concentrated residue was applied on chromatotron (silica) eluted with hexane-chloroform to afford two components [R_{f}s=0.28 (minor) and 0.22 (major) in chloroform] respectively. The major one is the compound based on the proton NMR. It is a yellowish solid (26 mg, Y=31%).

### Compound 43. 4-(4-Chloro-phenyl)-thiazole-2-carboxylic acid 3-trifluoromethoxy-benzylamide (B250115)

4-(4-Chloro-phenyl)-thiazole-2-carboxylic acid (63 mg, 0.23 mmol) was dissolved in THF (5 mL), followed by addition of CDI (56 mg, 0.35 mmol). The slurry mixture was stirred at RT for 1 hour. 3-(Trifluoromethoxy)benzylamine (53 mg, 0.28 mmol) dissolved in THF (2 mL) was added to it. The reaction was continued at RT overnight. After removal of the solvent, the residue was washed twice with water (2×10 mL). After filtration, the residue was dissolved in dichloromethane (40 mL). It was washed with 0.5 N HCl (20 mL), 5%NaHCO₃ (20 mL) and water (20 mL). The organic solution was dried over sodium sulfate After filtration through a pad of silica eluted with chloroform and concentration, the residue was applied on chromatotron (silica) eluted with hexane-chloroform to afford the product (R_{f}=0.19, chloroform). (38 mg, Y=40%)

### Compound 44. 4-(4-Chloro-phenyl)-thiazole-2-carboxylic acid [2-(3-phenoxy-phenyl)-ethyl]-amide (B250116)

4-(4-Chloro-phenyl)-thiazole-2-carboxylic acid (75 mg, 0.27 mmol) was dissolved in THF (5 mL), followed by addition of CDI (58 mg, 0.36 mmol). The slurry mixture was stirred at RT for 1 hour. 3-Phenoxyphenethlylamine (72 mg, 0.34 mmol) dissolved in THF (2 mL) was added to it. The reaction was continued at RT overnight. After removal of the solvent, the residue was washed twice with water (2×10 mL). After filtration, the residue was dissolved in dichloromethane (40 mL). It was washed with 0.5 N HCl (20 mL), 5% NaHCO₃ (20 mL) and water (20 mL). The organic solution was dried over sodium sulfate. R_{f} value (chloroform, Silica) was 0.25. After filtration through a pad of silica eluted with chloroform and concentration, the residue was applied on chromatotron (silica) eluted with hexane-chloroform to afford the product. It was a yellowish solid (41 mg, Y=35%).

### Compound 45, 4-(4-Chloro-phenyl)-thiazole-2-carboxylic acid [2-(1-methyl-pyrrolidin-2-yl)-ethyl]-amide (B250120)

2-(1-Methyl-pyrrolidin-2-yl)-ethylamine (37 mg, 0.29 mmol) was dissolved in dichloromethane (5 mL), followed by addition of 4-(4-Chloro-phenyl)-thiazole-2-carboxylic acid hydrochloride salt (80 mg, 0.29 mmol), HOBt (53 mg, 0.39 mmol), EDC [N-(3-dimethylaminopropyl)-N-ethylcarbodiimide hydrochloride] (72 mg, 0.38 mmol) and diisopropylethylamine (70 uL). The mixture was stirred at RT for two days. After diluted with dichloromethane (30 mL), the mixture was washed with water (30 mL). The water solution was extracted with dichloromethane (30 mL,). The combined dichloromethane was washed with water (30 mL) and dried over sodium sulfate. After passing through a pad of alumina eluted with 1% MeOH in chloroform and concentration, the residue was purified by chromatotron (alumina) eluted with hexane-chloroform to afford a yellow semisolid (70 mg, Y=69%). R_{f}═0.33 (alumina, 1% MeOH in chloroform). The structure of the compound was confirmed by NMR and MS.

### Compound 46. 4-(4-Chloro-phenyl)-thiazole-2-carboxylic acid (4-methyl-piperazin-1-yl)-amide (B250121)

4-(4-Chloro-phenyl)-thiazole-2-carboxylic acid (99 mg, 0.36 mmol) was dissolved in THF (5 mL), followed by addition of CDI (77 mg, 0.48 mmol). The slurry mixture was stirred at RT for 1 hour. 1-Amino-4-methylpiperazine (47 mg, 0.36 mmol) dissolved in THF (2 mL) was added to it. The reaction was continued at RT for 18 hours After removal of the solvent, the residue was dissolved in dichloromethane and directly passed through a pad of neutral alumina column eluted with 1% MeOH in chloroform. After concentration, the residue was applied on chromatotron (alumina) eluted with hexane-chloroform to afford a white solid (25 mg, Y=21%). It was the compound based on NMR and MS. R_{f}═0.42 (MeOH:chloroform:ammonium hydroxide 1:9:0.5, Silica); R_{f}═0.39 (1% MeOH in chloroform, alumina).

### Compound 47. N-[4-(4-Chloro-phenyl)-thiazol-2-yl]-3-(2,4-difluoro-phenyl)-propionamide (B250222)

3-(2,4-Difluorophenyl)propionic acid (186 mg, 1.0 mmol) was dissolved in dichloromethane (10 mL). DMF (50 μL) and 2 M oxalyl chloride in CH₂Cl₂ (0.7 mL) were added to it at RT. After stirring for 0.5 hour, the solvent was removed ill vacuo. The residue was dissolved in dioxane (10 mL). 2-Amino-4-(4-chlorophenyl)thiazole (211 mg, 1.0 mmol) was added to it, followed by addition of pyridine (200 uL). The mixture was heated at boiling for 0.5 hour. After cooling down, the solvent was removed in vacuo. The residue was dissolved in EtOAc (50 mL,) and washed with water (50 mL). The EtOAc solution was dried over sodium sulfate. After filtration through a pad of silica and concentration, the residue was purified by chromatotron (silica) eluted with CHCl₃ to afford the product. With R_{f}=0.40. (139 mg, Y=37%) NMA confirmed it was the target compound.

### Compound 48. 4-(4-Chloro-phenyl)-thiazole-2-carboxylic acid (2-ethylsulfanyl-ethyl)-amide (B250223)

4-(4-Chloro-phenyl)-thiazole-2-carboxylic acid (51 mg, 0.1 8 mmol) was dissolved in THE (5 mL), followed by addition of CDI (34 mg, 0.21 mmol). The slurry mixture was stirred at RT for 1 hour, 2-(Ethylthio)ethylamine (25 mg, 0.24 mmol) dissolved in THF (2 mL) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with chloroform to afford two components. R_{f} values (chloroform) were (1) 0.24, (2) major 0.12 which is the target compound, an off-white semi solid (47 mg, Y=80%). The structure of the compound was confirmed by NMR.

### Compound 49. 4-(4-Chloro-phenyl)-thiazole-2-carboxylic acid 2-fluoro-4-trifluoromethyl-benzylamide (B250224)

4-(4-Chloro-phenyl)-thiazole-2-carboxylic acid (53 mg, 0.19 mmol) was dissolved in THF (5 mL), followed by addition of CDI (37 mg, 0.23 mmol). The slurry mixture was stirred at RT for 1 hour, 2-Fluoro-4-(trifluoromethyl)benzylamine (43 mg, 0.22 mmol) dissolved in THF (2 mL) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with hexane-chloroform to afford one major component, R_{f} value (chloroform, Silica) was 0.26. It is a white solid (62 mg, Y=79%). The structure of the compound was confirmed by NMR.

### Compound 50. 4-(4-Chloro-phenyl)-thiazole-2-carboxylic acid (3,5-difluoro-phenyl)-amide (B250225)

4-(4-Chloro-phenyl)-thiazole-2-carboxylic acid (52 mg, 0.19 mmol) was dissolved in THF (5 mL), followed by addition of CDI (38 mg, 0.23 mmol). The slurry mixture was stirred at RT for 1 hour. 3,5-Difluoroaniline (29 mg, 0.22 mmol) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with hexane-chloroform to afford the product. R_{f} value (chloroform, silica) was 0.55. It is a white solid (32 mg, Y=48%). The structure of the compound was confirmed by NMR,

### Compound 51. 4-(4-Chloro-phenyl)-thiazole-2-carboxylic acid 4-methylsulfanyl-benzylamide (B250301)

4-(4-Chloro-phenyl)-thiazole-2-carboxylic acid (52 mg, 0.19 mmol) was dissolved in THF (5 mL,), followed by addition of CDI (35 mg, 0.22 mmol). The slurry mixture was stirred at RT for 1 hour 4-(Methylthio)benzylamine (34 mg, 0.22 mmol) dissolved in THF (2 mL) was added to it. The reaction was continued at RT for 20 hours After removal of the solvent, the residue was dissolved in dichloromethane. After filtration through a pad of silica eluted with chloroform and concentration, the residue was applied on chromatotron (silica) eluted with chloroform to afford the compound with R_{f}=0.15 (chloroform, Silica) It was an off-white semisolid (47 mg, Y=80%). The structure of the compound was confirmed by NMR.

### Compound 52. 4-(4-Chloro-phenyl)-thiazole-2-carboxylic acid 4-trifluoromethoxy-benzylamide (B250302)

4-(4-Chloro-phenyl)-thiazole-2-carboxylic acid (51 mg, 0.18 mmol) was dissolved in THF (5 mL), followed by addition of CDI (34 mg, 0.21 mmol). The slurry mixture was stirred at RT for 1 hour. 4-(Trifluoromethoxy)benzylamine (42 mg, 0.22 mmol) dissolved in THF (2 mL) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with chloroform to afford the product with R_{f}═0.22 (chloroform, silica). It was an off-white semisolid (42 mg, Y=56%). The structure of the compound was confirmed by NMR and MS.

### Compound 53. 4-(4-Chloro-phenyl)-thiazole-2-carboxylic acid 4-fluoro-3-trifluoromethyl-benzylamide (B250303)

4-(4-Chloro-phenyl)-thiazole-2-carboxylic acid (51 mg, 0.18 mmol) was dissolved in THF (5 mL), followed by addition of CDI (37 mg, 0.23 mmol). The slurry mixture was stirred at RT for 1 hour. 4-Fluoro3-(trifluoromethyl)benzylamine (45 mg, 0.23 mmol) dissolved in THF (2 mL) was added to it. The reaction was continued at RT for 24 hours. After removal of the solvent, the residue was dissolved in dichloromethane. After filtration through a pad of silica eluted with chloroform, the concentrated residue was applied on chromatotron (silica) eluted with chloroform to afford a component (R_{f}=0.25, chloroform, silica). It was an off-white semisolid (47 mg, Y=80%). The structure of the compound was confirmed by NMR.

### Compound 54. 4-(4-Chloro-phenyl)-thiazole-2-carboxylic acid 4-phenoxy-benzylamide (B250307)

4-(4-Chloro-phenyl)-thiazole-2-carboxylic acid (49 mg, 0.18 mmol) was dissolved in THF (5 mL), followed by addition of CDI (34 mg, 0.21 mmol). The slurry mixture was stirred at RT for 1 hour. 4-Phenoxybenzylamine (41 mg, 0.21 mmol) dissolved in THF (2 mL) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with chloroform to afford one major component with R_{f}=0.19 (chloroform, silica). It was an off-white semisolid (30 mg, Y=40%). The structure of the compound was confirmed by NMR and MS.

### Compound 55. 4-(4-Chloro-phenyl)-thiazole-2-carboxylic acid (biphenyl-4-ylmethyl)-amide (B250308)

4-(4-Chloro-phenyl)-thiazole-2-carboxylic acid (53 mg, 0.19 mmol) was dissolved in THF (5 mL), followed by addition of CDI (37 mg, 0.23 mmol). The slurry mixture was stirred at RT for 1 hour. 4-Phenylbenzylamine (40 mg, 0.22 mmol) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with chloroform to afford one major component. R_{f} value (chloroform, silica) was 0.24. It was a yellowish solid (40 mg, Y=52%). The structure of the compound was confirmed by NMR and MS

### Compound 56. 4-(4-Chloro-phenyl)-thiazole-2-carboxylic acid [1-(4-chloro-phenyl)-ethyl]-amide (B250309)

4-(4-Chloro-phenyl)-thiazole-2-carboxylic acid (49 mg, 0.18 mmol) was dissolved in THF (5 mL), followed by addition of CDI (34 mg, 0.21 mmol). The slurry mixture was stirred at RT for 1 hour, 1-(4-Chlorophenyl)ethylamine (34 mg, 0.22 mmol) in THE (2 mL) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with chloroform to afford one major component. R_{f} value (chloroform, Silica) was 0.27. It was a semisolid, 21 mg, Y=31%. The structure of the compound was confirmed by the NMR and MS.

### Compound 57. 4-(4-Chloro-phenyl)-thiazole-2-carboxylic acid (3-tert-butylamino-propyl)-amide (B250310)

4-(4-Chloro-phenyl)-thiazole-2-carboxylic acid (51 mg, 0.18 mmol) was dissolved in THF (5 mL), followed by addition of CDI (36 mg, 0.22 mmol). The slurry mixture was stirred at RT for 1 hour. 3-tert-Butylaminopropylamine (29 mg, 0.22 mmol) in THF (2 mL) was added to it. The reaction was continued at RT for 24 hours. After removal of the solvent, the residue was dissolved in dichloromethane and passed through a small alumina (n) column eluted with 3% MeOH in chloroform. After concentration, the residue was applied on chromatotron [alumina (n)] eluted with 2% MeOH in chloroform to afford one major component. R_{f} value [chloroform, alumina(n)] was 0.10. It was a yellow oil (52 mg, Y=82%). The structure of the compound was confirmed by NMR and MS.

### Compound 58. 4-(4-Chloro-phenyl)-thiazole-2-carboxylic acid 4-trifluoromethylbenzylamide (B250311)

4-(4-Chloro-phenyl)-thiazole-2-carboxylic acid (48 mg, 0.17 mmol) was dissolved in THF (5 mL), followed by addition of CDI (33 mg, 0.20 mmol). The slurry mixture was stirred at RT for 1 hour. 4-Trifluoromethyl-benzylamine (36 mg, 0.21 mmol) in THE (2 ml) was added to it. The reaction was continued at RT for 24 hours. After removal of the solvent, the residue was dissolved in dichloromethane. After filtration through a pad of silica eluted with chloroform and concentration, the residue was applied on chromatotron (silica) eluted with chloroform to afford one major component. R_{f} value (chloroform, silica) was 0.27. It was a white solid powder (47 mg, Y=70%). The structure of the compound was confirmed by the NMR and MS.

### Compound 59. 4-(4-Chloro-phenyl)-thiazole-2-carboxylic acid (3-pyrrolidin-1-yl-propyl)-amide (B250314)

4-(4-Chloro-phenyl)-thiazole-2-carboxylic acid (51 mg, 0.18 mmol) was dissolved in THF (5 mL), followed by addition of CDI (35 mg, 0.22 mmol). The slurry mixture was stirred at RT for 1 hour, 3-(1-Pyrrolidino)propylamine (29 mg, 0.23 mmol) in THF (2 mL) was added to it. The reaction was continued at RT for 24 hours. After removal of the solvent, the residue was dissolved in dichloromethane and passed through a small alumina (n) column eluted with 1% MeOH in chloroform. After concentration, the residue was applied on chromatotron [alumina (n)] eluted with chloroform to afford one major component. R_{f} value [1% MeOH in chloroform, alumina (n)] was 0.35. It was a light yellow solid powder (38 mg, Y=60%). The structure of the compound was confirmed by NMR and MS.

### Compound 60. 4-(4-Chloro-phenyl)-thiazole-2-carboxylic acid 3,5-bis-trifluoromethyl-benzylamide (B250315)

4-(4-Chloro-phenyl)-thiazole-2-carboxylic acid (49 mg, 0.18 mmol) was dissolved in THF (5 mL), followed by addition of CDI (34 mg, 0.21 mmol). The slurry mixture was stirred at RT for 1 hour. 3,5-Bis-trifluoromethyl-benzylamine (51 mg, 0.21 mmol) was added to it The reaction was continued at RT for 24 hours. After removal of the solvent, the residue was dissolved in dichloromethane. After filtration through a pad of silica eluted with chloroform and concentration, the residue was applied on chromatotron (silica) eluted with chloroform to afford one major component. R_{f} value (chloroform, silica) was 0.30. It was a semisolid (58 mg, Y=69%). The structure of the compound was confirmed by NMR and MS.

### Compound 61. 4-(4-Chloro-phenyl)-thiazole-2-carboxylic acid (2-pyridin-4-yl-ethyl)-amide (B250316)

4-(4-Chloro-phenyl)-thiazole-2-carboxylic acid (51 mg, 0.18 mmol) was dissolved in THF (5 mL), followed by addition of CDI (35 mg, 0.22 mmol). The slurry mixture was stirred at RT for 1 hour, 4-(2-Aminoethyl)pyridine (26 mg, 0.21 mmol) in THF (2 mL) was added to it. The reaction was continued at RT for 24 hours. After removal of the solvent, the residue was dissolved in dichloromethane and passed through a small alumina (n) column eluted with chloroform. After concentration, the residue was purified by chromatotron [alumina (n)] eluted with chloroform to afford one major component, a light yellow solid powder (20 mg, Y=32%) R_{f} value [chloroform, alumina(n)] was 0.18.

¹H NMR (CDCl₃) δ 8.56 (d, J=5.3 Hz, 2 H), 7.80 (d, J=8.4 Hz, 2 H), 7.70 (s, 1 H), 7.42 (d, J=84 Hz, 2 H), 7.21 (d, J=5.3 Hz, 2 H), 3.78 (dt, J=6.9 Hz, J=6.9 Hz, 2 H), 3.00 (t, J=6.9 Hz, 2 H); ¹³C NMR (CDCl₃) δ 163.1 , 159.3, 155.0, 149.9 (2 C), 147.3, 134.5, 131.9, 129.0 (2 C), 127.5 (2 C), 124.0 (2 C), 118.5, 39.9, 35.2; MS (MALDI-TOF) m/z calcd for C₁₇H₁₄ClN₃OS (M+H⁺) 344, found 344.

### Compound 62. 4-(4-Chloro-phenyl)-thiazole-2-carboxylic acid (1H-tetrazol-5-yl)-amide (B250317)

4-(4-Chloro-phenyl)-thiazole-2-carboxylic acid (49 mg, 0.18 mmol) was dissolved in THF (5 mL), followed by addition of CDI (34 mg, 0.21 mmol). The slurry mixture was stirred at RT for 1 hour. 5-Aminotetrazole (19 mg, 0.22 mmol) was added to it. The reaction was continued at RT for 24 hours. After removal of the solvent, the residue was washed twice with ether and then water. After filtration and drying, an off-white solid was obtained (18 mg, Y=33%). Note: No good TLC result was observed

¹H NMR (DMSO-d₆) δ 8.66 (s 1 H), 8.26 (d, J=8.4 Hz, 2 H), 7.57 (d=8.4 Hz, 2 H); ¹³C NMR (DMSO-d₆) δ 160.1, 158.0, 154.4, 150.0, 133.2, 131.9, 128.7 (2 C), 128.2 (2 C), 121.8; MS (MALDI-TOF) m/z calcd for C₁₁H₁₈ClN₆OS (M+H⁺) 307, found 307.

### Compound 63. 4-(4-Chloro-phenyl)-thiazole-2-carboxylic acid 4-methanesulfonyl-benzylamide (B1250318)

4-(4-Chloro-phenyl)-thiazole-2-carboxylic acid hydrochloride salt (50 mg, 0.18 mmol) and HOBt (42 mg, 0.31 mmol) were suspended in dichloromethane (5 mL), followed by addition of EDC [N-(3-dimethylaminopropyl)-N-ethylcarbodiimide hydrochloride] (52 mg, 0.27 mmol) and diisopropylethylamine (110 uL). After 10 min stirring, 4-methylsulfonylbenzylamine hydrochloride (46 mg, 0.21 mmol) was added to it. The mixture was stirred at RT for 12 hours. After diluted with dichloromethane (30 mL), the mixture was washed with water (30 mL). The dichloromethane was dried over sodium sulfate. After passing through a pad of silica eluted with 1% MeOH in chloroform and concentration, the residue was purified by chromatotron (silica) eluted with hexane-chloroform to afford a yellow semisolid (10 mg, Y=14%). R_{f}=0.14 (Silica, 1% MeOH in chloroform). The structure of the compound was confirmed by NMR and MS.

### Compound 64. 4-(4-Chloro-phenyl)-thiazole-2-carboxylic acid (2-benzo[1,3]dioxol-5-yl-ethyl)-amide (B250321)

4-(4-Chloro-phenyl)-thiazole-2-carboxylic acid hydrochloride salt (50 mg, 0.18 mmol), HOBt (42 mg, 0.31 mmol) were suspended in dichloromethane (5 mL), followed by addition of EDC (52 mg, 0.27 mmol) and diisopropylethylamine (110 uL). The mixture became a solution. 3,4-Methylenedioxyphenethylamine (42 mg, 0.21 mmol) was added to it after 10 min. The mixture was stirred at RT for 12 hours. After diluted with dichloromethane (30 mL), the mixture was washed with water (30 mL). The dichloromethane was dried over sodium sulfate. After passing through a pad of silica eluted with chloroform and concentration, the residue was purified by chromatotron (silica) eluted with hexane-chloroform to afford the compound (13 mg, Y=19%). The structure of the compound was confirmed by NMR and MS.

### Compound 65. N-[4-(4-Chloro-phenyl)-thiazol-2-yl]3-fluoro-benzamide (B250322)

3-Fluorobenzoyl chloride (160 mg, 110 mmol) was dissolved in dioxane (10 mL,). 2-Amino-4-(4-chlorophenyl)thiazole (212 mg, 1.0 mmol) was added to it, followed by addition of pyridine (150 uL). The mixture was stirred at RT for two hours and then heated and kept boiling for 30 min. After cooling down to RT, the solvent was removed in vacuo. The residue was dissolved in EtOAc (50 mL) and washed with water (50 mL). The EtOAc solution was dried over sodium sulfate. After filtration through a pad of silica and concentration, the residue was purified by chromatotron (silica) eluted with hexane-CHCl₃ to afford a white solid product (177 mg, Y=53%). NMR and MS confirmed it was the compound.

### Compound 66 N-[4-(4-Chloro-phenyl)-thiazol-2-yl]-2-fluoro-4-trifluoromethyl-benzamide (B250323)

2-Fluoro-4-trifluoromethyl-benzoyl chloride (228 mg, 1.0 mmol) was dissolved in dioxane (10 mL). 2-Amino-4-(4-chlorophenyl)thiazole (212 mg, 1.0 mmol) was added to it, followed by addition of pyridine (150 uL). The mixture was stirred at RT for two hours and then heated and kept boiling for 30 min. After cooling down to RT, the solvent was removed in vacuo. The residue was dissolved in EtOAc (50 mL) and washed with water (50 mL). The EtOAc solution was dried over sodium sulfate. After filtration through a pad of silica and concentration, the residue was purified by chromatotron (silica) eluted with hexane-CHCl₃ to afford the compound (190 mg, Y=47%), NMR and MS confirmed it was the compound.

### Compound 67. N-[4-(4-Chloro-phenyl)-thiazol-2-yl]-4-fluoro-benzamide (B250324)

4-Fluorobenzoyl chloride (161 mg, 1.01 mmol) was dissolved in dioxane (10 mL). 2-Amino-4-(4-chlorophenyl)thiazole (214 mg, 1.01 mmol) was added to it, followed by addition of pyridine (150 uL). The mixture was stirred at RT for two hours and then heated and kept boiling for 30 min. After cooling down to RT, the solvent was removed in vacuo. The residue was dissolved in EtOAc (50 mL) and washed with water (50 mL). The EtOAc solution was dried over sodium sulfate. After filtration through a pad of silica and concentration, the residue was purified by chromatotron (silica) eluted with hexane-CHCl₃ to afford the compound (146 mg, Y=43%). NMR confirmed it was the compound-.Compound 68. 2,4-Dichloro-N-[4-(4-chloro-p

2,4-Dichlorobenzoyl chloride (209 mg, 1.0 mmol) was dissolved in dioxane (10 mL). 2-Amino-4-(4-chlorophenyl)thiazole (211 mg, 1.0 mmol) was added to it, followed by addition of pyridine (150 uL). The mixture was stirred at RT for two hours and then heated and kept boiling for 30 min. After cooling down to RT, the solvent was removed in vacuo. The residue was dissolved in chloroform (50 mL) and washed with water (50 mL). The water solution was extracted with chloroform (50 mL). The chloroform solution was dried over sodium sulfate. After filtration through a pad of silica and concentration, the residue was purified by chromatotron (silica) eluted with hexane-CHCl₃ to afford a solid product (R_{f}=0.45, chloroform) (219 mg, Y=57%). NMR and MS confirmed it was the compound.

### Compound 69. 2-Chloro-N-[4-(4-chloro-phenyl)-thiazol-2-yl]-2-phenyl-acetamide (B250329)

2-Chloro-2-phenylacetyl chloride (189 mg, 1.0 mmol) was dissolved in dioxane (10 mL), 2-Amino-4-(4-chlorophenyl)thiazole (211 mg, 1.0 mmol) was added to it, followed by addition of pyridine (150 uL). The mixture was stirred at RT for two hours and then heated and kept boiling for 30 min. After cooling down to RT, the solvent was removed in vacuo. The residue was dissolved in chloroform (50 mL) and washed with water (50 mL). The water solution was extracted with chloroform (50 mL). The combined chloroform solution was dried over sodium sulfate. After filtration through a pad of silica and concentration, the residue was purified by chromatotron (silica) eluted with hexane-CHCl₃ to afford a solid product (R_{f}=0.36, chloroform) (76 mg, Y=21%). NMR and MS confirmed it was the compound.

### Compound 70, N-[4-(4-Chloro-phenyl)-thiazol-2-yl]-2-(4-fluoro-phenyl)-acetamide (B250330)

4-Fluorophenylacetyl chloride (186 mg, 1.08 mmol) was dissolved in dioxane (10 mL). 2-Amino-4-(4-chlorophenyl)thiazole (228 mg, 1.08 mmol) was added to it, followed by addition of pyridine (150 uL). The mixture was stirred at RT for two hours and then heated and kept boiling for 30 min. After cooling down to RT, the solvent was removed in vacuo. The residue was dissolved in chloroform (50 mL) and washed with water (50 mL). The water solution was extracted with chloroform (50 mL). The combined chloroform solution was dried over sodium sulfate. After filtration through a pad of silica and concentration, part of the residue, which is not very soluble in chloroform, even in 5% MeOH in chloroform, was purified by chromatotron (silica plate) eluted with hexane-CHCl₃ to afford a solid product (R_{f}=0.18, chloroform) [12 mg+18 mg (impure)].

¹H NMR (DMSO-d₆) δ 7.90 (d, J=8.7 Hz, 2 H), 7.68 (s, 1 H), 7.48 (d, J=8.7 Hz, 2 H), 7.36 (dd, J=8.7 Hz, J=5.7 Hz, 2 H), 7.16 (dd, J=8.7 Hz, J=8.7 Hz, 2 H), 3.78 (s, 2 H); ¹³C NMR (DMSO-d₆) δ 169.2, 161.0 (d, J=240 Hz, 1 C), 157.8, 147.4, 132.9, 132.1, 131.0 (d, J=8.0 Hz, 2 C), 130.9, 128.6 (2 C), 127.2 (2 C), 115.0 (d, J=21 Hz, 2 C), 108.7, 40.7; MS (MALDI-TOP) m/z calcd for C₁₇H₁₂ClFN₂OS (M+H⁺) 347, found 347.

### Compound 71. [4-(4-Chloro-phenyl)-thiazol-2-yl]-bis-(3-phenyl-propyl)-amine (B240331A)

60% NaH (226 mg, 5.7 mmol), 2-amino-4-(4-chlorobenzo)thiazole (420 mg, 2 mmol) and K₂CO₃ (576 mg, 4.2 mmol) were suspended in DMF (10 mL) under Ar. After stirring at RT for 10 min, 3-bromo-1-phenylpropane (883 mg, 4.43 mmol) was added by syringe. The reaction was carried out at RT for one hour, then at 70° C. (oil bath) for 20 hours. After cooling to RT, the reaction was quenched with ice. Water (30 mL) was added to it. The mixture was extracted with CHCl₃ (2×30 mL). The collected organic solution was washed with water (2×40 mL), which was dried over Na₂SO₄. After filtration and concentration, the residue was applied on a silica column eluted with 5%-10% EtOAc in hexane to afford two compounds, B3240331A (R_{f}=0.59, silica, 15% ethyl acetate in hexane) and B2403313B (R_{f}=0.20, silica, 15% ethyl acetate in hexane).

B240331A (17 mg, 0.04 mmol, Y=2%); ¹H NMR(CDCl₃) δ 7.72 (d, J=8.4 Hz, 2 H), 7.10-7.48 (m, 12 H, 6.64 (s, 1 H), 3.47 (t, J=75 Hz, 4 H), 2.67 (t, J=7.5 Hz, 4 H), 1.88-2.20 (m, 4 H); ¹³C NMR (CDCl₃) δ 169,5, 150.4, 141.2, 133.6, 132.8, 128.4, 128.3, 127.2, 125.9, 100.2, 51.0 (2 C), 33.2 (2 C), 28.8 (2 C); MS (MALDI-TOF) m/z calcd for C₂₇H₂₈ClN₂S (M+H⁺) 447, found 447.

### Compound 73. Benzyl-[4-(4-chloro-phenyl)-thiazol-2-yl]-amine (B240406B)

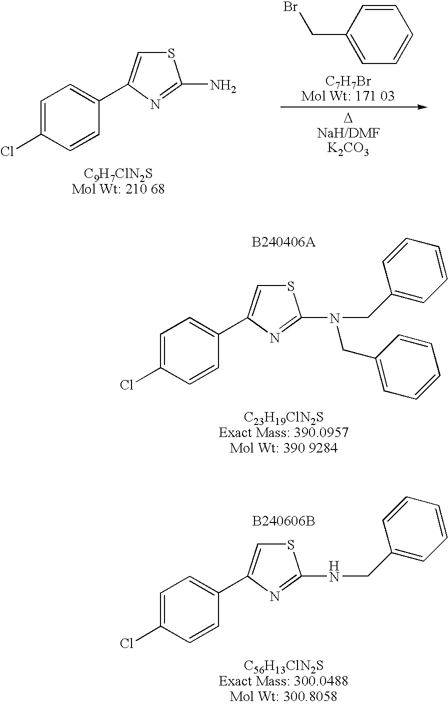

60% NaH (188 mg, 4.7 mmol), 2-amino4-(4-chlorophenyl)thiazole (421 mg, 2 mmol) and K₂CO₃ (552 mg, 4.0 mmol) were suspended in DMF (10 mL) under Ar. After stirring at RT for 10 min, benzyl bromide (819 mg, 4.8 mmol) was added by syringe. The reaction was carried out at RT for one hour, then at 70° C. (oil bath) for 15 hours After cooling down to RT, the mixture was transferred to a separator funnel with water (30 mL). The mixture was extracted with CHCl₃ (2×30 mL). The organic solution was washed with water (40 mL) and dried over Na₂SO₄. After filtration and concentration, the residue was purified by chromatography eluted with 5% ethyl acetate-10% ethyl acetate in hexane to afford three components B240406A (65 mg), B240406B (30 mg) and B240406C (30 mg), Compound 240406A (R_{f}=0.57, silica, 15% ethyl acetate in hexane); Compound 240406B (R_{f}═0.25, silica, 15% ethyl acetate in hexane); Compound 240406C (R_{f}=0.21, silica, 15% ethyl acetate in hexane). The structures of the compounds were confirmed by both NMR and MS.

### Compound 87. N-(5-Chloro-benzooxazol-2-yl)-2-nitro-benzamide (B231106)

2-Nitrobenzoyl chloride (124 mg, 0.67 mmol) was placed in a dried 100 mL round bottom reaction flask. Anhydrous benzene (5 mL) was added to it, followed by addition of 2-amino-5-chlorobenzoxazole (113 mg, 0.67 mmol) and TEA (150 uL). The mixture was stirred at RT for 0.5 h. It was then heated under reflux for 1.5 h. After cooling to RT and removal of the solvent, the residue was crystallized in 95% ethanol to afford a yellow solid (114 mg). Y=54%. NMR and MS confirmed it is the target compound,

### Compound 89. N-(5-Chloro-benzooxazol-2-yl)3-(4-nitro-phenyl)-acrylamide (B231110)

Trans-4-nitrocinnaoyl chloride (100 mg, 0.50 mmol) was placed in a dried 100 mL round bottom reaction flask. Anhydrous benzene (5 mL) was added to it, followed by addition of 2-amino-5-chlorobenzoxazole (85 mg, 0.50 mmol) and TEA (100 uL). The mixture was stirred at RT for 0.5 h. It was then heated under reflux for 1 h. After cooling to RT and removal of the solvent, the residue was partially soluble in hot 95% ethanol to afford an orange powder solid (30 mg). The filtrate was concentrated in air and crystallized at RT to afford second part of orange solid (33 mg), Yₜₒₜₐₗ =37%, NM and MS confirmed it is the target compound.

### Compound 90. Undec-10-enoic acid (5-chloro-benzooxazol-2-yl)-amide (B231117)

10-Undecenoyl chloride (120 mg, 0.59 mmol) was placed in a dried 100 mL round bottom reaction flask by syringe. Anhydrous benzene (5 mL) was added to it, followed by addition of 2-amino-5--chlorobenzoxazole (100 mg, 0.59 mmol) and TEA (100 uL). The mixture was stirred at RT for 0.5 hi It was then heated under reflux for 2 h. After cooling to RT and removal of the solvent, the residue was crystallized in ethanol to afford a white powder solid (65 mg, 0.19 mmol). Y=33%. NMR and MS confirmed it is the target compound.

### Compound 91. Tetradecanoic acid (5-chloro-benzooxazol-2-yl)-amide (B231119)

Myristoyl chloride (152 mg, 0.62 mmol) was placed in a dried 100 mL round bottom reaction flask by syringe. Anhydrous benzene (5 mL) was added to it, followed by addition of 2-amino-5-chlorobenzoxazole (104 mg, 0.62 mmol) and TEA (100 uL) The mixture was stirred at RT for 0.5 h. It was then heated under reflux for 2 h. After cooling to RT and removal of the solvent, the residue was crystallized in ethanol to afford a grey powder solid (95 mg, 0.25 mmol). Y=40%. NMR and MS confirmed it is the target compound.

### Compound 92. Hexadecanoic acid (5-chloro-benzooxazol-2-yl)-amide (B1231124)

Palmitoyl chloride (153 mg, 0.56 mmol) was placed in a dried 100 mL round bottom reaction flask by syringe. Anhydrous benzene (5 mL) was added to it followed by addition of 2-amino-5-chlorobenzoxazole (95 mg, 0.56 mmol) and TEA (100 uL). The mixture was stirred at RT for 0.5 h. It was then heated under reflux for 2 h. After cooling to RT and removal of the solvent, the residue was crystallized in ethanol to afford a grey powder solid (94 mg, 0.23 mmol). Y=41%. NMR and MS confirmed it is the target compound,

### Compound 95. Butyric acid 4-[(6-chloro-benzothiazol-2-ylcarbamoyl)-methyl]-2-methoxy-phenyl ester (B241019)

4-Butanoyl3-methoxyphenylacetic acid (90 mg, 0.36 mmol) was dissolved in dichloromethane (10 mL). DMF (50 μL) and 2 M oxalyl chloride in CH₂Cl₂ (0.4 mL) were added to it at RT. After 0.5 hour stirring, the solvent was removed in vacuo. The residue was dissolved in dioxane (10 mL). 2-Amino-6-chlorobenzothiazole (93 mg, 0.5 mmol) was added to it, followed by addition of pyridine (60 uL). The reaction was carried out at RT for 0.5 hour and then at 100° C. for 1 hour. The mixture became light yellow. After cooling down to RT, the solvent was removed in vacuo. The residue was dissolved in EtOAc (30 mL) and washed with water (30 mL). The EtOAc solution was dried over sodium sulfate. After filtration and concentration, the residue was purified by Silica-chromatography eluted with CHCl₃. The collected fractions were concentrated and purified again by chromatotron (silica) eluted with hexane-CHCl₃ to afford a solid product (70 mg, Y=46%).

¹H NMR (CDCl₃) δ 9.20 (bs, 1 H), 7.79 (d, J=2.1 Hz, 1 H), 7.63 (d, J=8.7 Hz, 1 H), 7.38 (d, J=8.7 Hz, J=2.1 Hz, 1 H), 7.06 (d, J=7.5 Hz, 1 H), 6.89 (s, 1 H), 6.88 (d, J=7.5 Hz, 1 H), 3.83 (s, 2 H), 3.82 (s, 3 H), 2.58 (t, d=7.5 Hz, 2 H), 1.72-1.88 (m, 2 H), 1.06 (t, d=7.5 Hz, 3 H); ¹³C NMR (CDCl₃) δ 171.5, 168.9, 157.9, 151.6, 146.6, 139.6, 133 3, 131.0, 129.6, 127.0, 123.6, 121.6 (2 C), 121.0, 113.4, 55.9, 43.4, 35.9, 18.6, 13.7; MS (MALDI-TOF) m/z calcd for C₂₀H₂₀ClN₂O₄S (M+H⁺) 419, found 419A

### EXAMPLE 3

### Assays for Inhibition of Human SK Activity

An assay for identifying inhibitors of recombinant human SK has been established (French et al., 2003, Cancer Res 63: 5962). cDNA for human SK was subcloned into a pGEX bacterial expression vector, which results in expression of the enzyme as a fusion protein with glutathione-S-transferase, and the fusion protein is then purified on a column of immobilized glutathione. SK activity is measured by incubation of the recombinant SK with [³H sphingosine and 1 mM ATP under defined conditions, followed by extraction of the assay mixture with chloroform : methanol under basic conditions. This results in the partitioning of the unreacted [³H]sphingosine into the organic phase, while newly synthesized [³H]S1P partitions into the aqueous phase. Radioactivity in aliquots of the aqueous phase is then quantified as a measure of [³H]S1P formation. There is a low background level of partitioning of [³ H]sphingosine into the aqueous phase, and addition of the recombinant SK greatly increases the formation of [³H]S1P. A positive control, DMS, completely inhibits SK activity at concentrations above 25 μM.

In an alternate assay procedure, the recombinant human SK was incubated with unlabeled sphingosine and ATP as described above. After 30 minutes, the reactions were terminated by the addition of acetonitrile to directly extract the newly synthesized S1P. The amount of S1P in the samples is then quantified as follows. C₁₇ base D-erythro-sphingosine and C₁₇ S1P are used as internal standards for sphingosine and S1P, respectively These seventeen-carbon fatty acid-linked sphingolipids are not naturally produced, making these analogs excellent standards. The lipids are then fractionation by High-Performance Liquid Chromatography using a C8-reverse phase column eluted with 1 mM methanolic ammonium formate/2 mM aqueous ammonium formate. A Finnigan LCQ Classic LC-MS/MS is used in the multiple reaction monitoring positive ionization mode to acquire ions at m/z of 300 (precursor ion)→282 (production) for sphingosine and 380→264 for S1P. Calibration curves are generated by plotting the peak area ratios of the synthetic standards for each sphingolipid, and used to determine the normalized amounts of sphingosine and S1P in the samples.

### EXAMPLE 4

### Inhibition of Human SK by Compounds of this Invention

Each Compound of this invention was tested for its ability to inhibit recombinant SK using the LC/MS/MS assay described above. Typically, the Compounds were individually dissolved in dimethylsulfoxide and tested at a final concentration of 6 micrograms/ml. The results for the assays are shown in Table 4. The data demonstrate that compounds of Formula I, II, III or IV demonstrate a range of abilities to inhibit the in vitro activity of recombinant SK. Several Compounds caused complete suppression of SK activity at the concentration of 6 micrograms/ml (corresponding to approximately 15 micromolar). As detailed in the Examples below, significant concentrations of the Compounds can be achieved in the blood of mice receiving the Compounds by oral administration, indicating that the Compounds are sufficiently potent to be therapeutically useful.

Although many of the Compounds inhibited the purified SK enzyme, it was useful to determine their abilities to inhibit endogenous SK in an intact cell. We have previously described an intact cell assay where, following treatment with a test compound, MDA-MB-231 human breast carcinoma cells are incubated with [³H]sphingosine at a final concentration of 1 μM (French et al., 2003, Cancer Res 63: 5962). The cells take up the exogenous [³H]sphingosine and convert it to [³H]S1P through the action of endogenous SK. The resulting [³H]S1P is isolated via charge-based separation as indicated above. The results from this assay are indicated in Table 4. The data demonstrate that many of the Compounds that inhibit purified SK also inhibit SK activity in the intact cell. For potency studies, MDA-MB-231 cells were exposure to varying concentrations of a test Compound and then assayed for conversion of [³H]sphingosine to [³H]S1P. Each of Compounds decreased [³H]S1P formation in a dose dependent fashion, with IC₅₀ values ranging from 5 to 34 μM. These results demonstrate that compounds of formula I, II, III or IV effectively inhibit SK activity in intact cells.

TABLE 4
Inhibition of SK activity. Human SK was incubated with 6 μg/ml
of the indicated compounds, and then assayed for activity as
described above. Values in the column labeled “Recombinant SK (%
inhibition)” represent the percentage of SK activity that was
inhibited. MDA-MB-231 cells were incubated with 20 μg/ml of the
indicated compounds and then assayed for endogenous SK activity as
indicated above. Values in the column labeled “Cellular S1P (%
inhibition)” represent the percentage of S1P production that was
inhibited. Additionally, MDA-MB-231 cells were treated with varying
concentration of certain compounds and the amount of S1P produced
by the cells was determined. Values in the column labeled
“Cellular S1P IC₅₀ (μM)” represent the
concentration of compound required to inhibit the production of
S1P by 50%. ND = not determined.

| | Recombinant SK | Cellular S1P | Cellular S1P |
|---|---|---|---|
| Compound | (% inhibition) | (% inhibition) | IC₅₀ (μM) |
| 1 | ND | 32 | ND |
| 2 | 30 | 43 | ND |
| 3 | 88 | 34 | ND |
| 4 | 53 | 39 | ND |
| 5 | 0 | 25 | ND |
| 6 | 0 | 21 | ND |
| 7 | 71 | 21 | ND |
| 8 | ND | 80 | 34 |
| 9 | ND | 32 | ND |
| 10 | 100 | 48 | ND |
| 11 | ND | 55 | ND |
| 12 | ND | 13 | ND |
| 13 | 0 | 0 | ND |
| 14 | 0 | 39 | ND |
| 15 | 73 | 23 | ND |
| 16 | ND | 83 | ND |
| 17 | ND | 57 | ND |
| 18 | ND | 65 | ND |
| 19 | 36 | 53 | ND |
| 20 | 6 | 62 | ND |
| 21 | 26 | 41 | ND |
| 22 | 34 | 33 | ND |
| 23 | 45 | 14 | ND |
| 24 | 0 | 69 | ND |
| 25 | 0 | 79 | ND |
| 26 | 0 | 79 | ND |
| 27 | ND | 68 | ND |
| 28 | 87 | 65 | ND |
| 29 | 0 | ND | ND |
| 30 | 0 | ND | ND |
| 31 | 58 | ND | ND |
| 32 | ND | ND | ND |
| 33 | ND | ND | ND |
| 34 | 0 | 28 | ND |
| 35 | 80 | 17 | ND |
| 36 | 14 | 0 | ND |
| 37 | 23 | 0 | ND |
| 38 | 75 | 0 | ND |
| 39 | 69 | 0 | ND |
| 40 | 56 | 0 | ND |
| 41 | 22 | 0 | ND |
| 42 | 79 | 0 | ND |
| 43 | 59 | 0 | ND |
| 44 | 69 | 0 | ND |
| 45 | 42 | 0 | ND |
| 46 | 80 | 0 | ND |
| 47 | 21 | ND | ND |
| 48 | 56 | ND | ND |
| 49 | 67 | ND | ND |
| 50 | 21 | ND | ND |
| 51 | 36 | ND | ND |
| 52 | 78 | ND | ND |
| 53 | 44 | ND | ND |
| 54 | 25 | ND | ND |
| 55 | 20 | ND | ND |
| 56 | 81 | ND | ND |
| 57 | 16 | ND | ND |
| 58 | 86 | ND | ND |
| 59 | 46 | ND | ND |
| 60 | 87 | ND | ND |
| 61 | 0 | ND | ND |
| 62 | 60 | ND | ND |
| 63 | 3 | ND | ND |
| 64 | 90 | ND | ND |
| 65 | 66 | ND | ND |
| 66 | 61 | ND | ND |
| 67 | 41 | ND | ND |
| 68 | 73 | ND | ND |
| 69 | 55 | ND | ND |
| 70 | 54 | ND | ND |
| 71 | 44 | 15 | ND |
| 72 | 79 | 27 | ND |
| 76 | ND | 81 | 5 |
| 74 | ND | ND | ND |
| 75 | 3 | ND | ND |
| 76 | 51 | ND | ND |
| 77 | 85 | ND | ND |
| 78 | 70 | ND | ND |
| 79 | 53 | ND | ND |
| 80 | ND | 70 | ND |
| 81 | ND | 14 | ND |
| 82 | ND | 67 | ND |
| 83 | ND | 55 | ND |
| 84 | ND | 76 | ND |
| 85 | ND | ND | ND |
| 86 | ND | ND | ND |
| 87 | ND | 64 | 22 |
| 88 | ND | 46 | ND |
| 89 | ND | 74 | 5.8 |
| 90 | ND | 39 | ND |
| 91 | ND | 0 | ND |
| 92 | ND | 4 | ND |
| 93 | ND | 53 | ND |
| 94 | ND | 13 | ND |
| 95 | ND | ND | ND |
| 96 | ND | 18 | ND |
| 97 | 71 | 38 | ND |

### EXAMPLE 5

### Selectivity of SK Inhibitors of this Invention

A common problem with previous attempts to develop protein kinase inhibitors is the lack of selectivity toward the target kinase since the majority of these compounds interact with nucleotide-binding domains that are highly conserved among kinases To determine if compound of this invention are non-selective kinase inhibitors, the effects of the SK inhibitor, Compound 73, on a diverse panel of 20 purified kinases was determined. The compound was tested at a single concentration of 50 μM. The kinases and the effects of the SK inhibitor are shown in Table 5.

The data indicate high specificity of Compound 73 for SK in that none of the 20 diverse kinases tested were significantly inhibited by this compound The panel included both serine/threonine kinases and tyrosine kinases, as well as several that are regulated by their interaction with lipids. Overall, the data indicate that the biological effects of the compounds of this invention are not mediated by off-target inhibition of protein kinases.

TABLE 5
Selectivity of Compound 73. Values represent the
percent of control activity of the indicated kinase
in the presence of 50 μM of Compound 73.

| Kinase | Compound 73 | Kinase | Compound 73 |
|---|---|---|---|
| Ca²⁺/calmodulin | 113 ± 3 | MEK | 104 ± 3 |
| PK IV | | kinase 1 | |
| Abl | 100 ± 1 | CHK1 | 124 ± 11 |
| Aurora-A | 100 ± 0 | EFGR | 99 ± 9 |
| Protein kinase C α | 92 ± 1 | Fyn | 102 ± 0 |
| Protein kinase C ε | 91 ± 2 | cSrc | 119 ± 6 |
| CDK1/cyclinB | 100 ± 0 | IKKα | 152 ± 7 |
| CDK2/cyclinE | 102 ± 7 | PKA | 94 ± 4 |
| P38 MAP kinase 1 | 88 ± 1 | PKBα | 102 ± 1 |
| P38 MAP kinase 2 | 101 ± 0 | PKBγ | 100 ± 10 |
| PDK1 | 98 ± 6 | cRaf | 104 ± 1 |

### EXAMPLE 6

### Cytotoxicity Profiles of SIC Inhibitors of this Invention

To further assess the biological efficacies of the Compounds in intact cells, each Compound was evaluated for cytotoxicity using human cancer cell lines. These experiments followed methods that have been extensively used. Cell lines tested included MCF-7 human breast adenocarcinoma cells. The indicated cell lines were treated with varying doses of the test Compound for 48 h. Cell survival was then determined using the SRB binding assay (Skehan et al., 1990, J Natl Cancer Inst 82: 1107), and the concentration of compound that inhibited proliferation by 50% (the IC₅₀) was calculated. The cytotoxicities of the compounds of this invention are summarized in Table 6. Values (in μM) represent the mean±sd for replicate trials. As the data show, the compounds of this invention are antiproliferative at low-micromolar concentrations. Overall, the data demonstrate that these Compounds are able to enter intact cells and prevent their proliferation, making them useful for the indications described above.

TABLE 6
Anticancer activity of compounds of this invention. The
cytotoxicity of the indicated Compounds toward human breast
cancer cells (MCF-7) was determined. Values represent the mean
IC₅₀ for inhibition of cell proliferation. ND = not determined.

| | MCF-7 |
|---|---|
| Compound | IC₅₀ (μM) |
| 1 | ND |
| 2 | 1.9 |
| 3 | 111 |
| 4 | 33 |
| 5 | 16 |
| 6 | 105 |
| 7 | 18 |
| 8 | 6.6 |
| 9 | 7.8 |
| 10 | 24 |
| 11 | 10 |
| 12 | 125 |
| 13 | 2.3 |
| 14 | 2.9 |
| 15 | 2.9 |
| 16 | 5.6 |
| 17 | 4 |
| 18 | 2.7 |
| 19 | ND |
| 20 | ND |
| 21 | ND |
| 22 | ND |
| 23 | ND |
| 24 | ND |
| 25 | ND |
| 26 | ND |
| 27 | ND |
| 28 | ND |
| 29 | 26 |
| 30 | 32 |
| 31 | ND |
| 32 | ND |
| 33 | ND |
| 34 | 38 |
| 35 | 130 |
| 36 | ND |
| 37 | ND |
| 38 | ND |
| 39 | ND |
| 40 | ND |
| 41 | ND |
| 42 | ND |
| 43 | ND |
| 44 | ND |
| 45 | ND |
| 46 | ND |
| 47 | ND |
| 48 | ND |
| 49 | ND |
| 50 | ND |
| 51 | ND |
| 52 | ND |
| 53 | ND |
| 54 | ND |
| 55 | ND |
| 56 | ND |
| 57 | ND |
| 58 | ND |
| 59 | ND |
| 60 | ND |
| 61 | ND |
| 62 | ND |
| 63 | ND |
| 64 | ND |
| 65 | ND |
| 66 | ND |
| 67 | ND |
| 68 | ND |
| 69 | ND |
| 70 | ND |
| 71 | 111 |
| 72 | 127 |
| 73 | 8.3 |
| 74 | 120 |
| 75 | ND |
| 76 | ND |
| 77 | ND |
| 78 | ND |
| 79 | ND |
| 80 | ND |
| 81 | ND |
| 82 | ND |
| 83 | ND |
| 84 | ND |
| 85 | ND |
| 86 | ND |
| 87 | 9.8 |
| 88 | 1.3 |
| 89 | 1.2 |
| 90 | 19 |
| 91 | 2.6 |
| 92 | 7.6 |
| 93 | ND |
| 94 | ND |
| 95 | ND |
| 96 | 24 |
| 97 | 1.7 |

### EXAMPLE 7

### Survey of Anticancer Activity of SK Inhibitors of this Invention

The data provided above demonstrate the abilities of compounds of this invention to inhibit the proliferation of human breast carcinoma cells. To examine the range of anticancer activity of representative compounds, the chemotherapeutic potencies of Compounds 8 and 73 towards a panel of varied human tumor cell lines representing several major tumor types were determined. The data are described in Table 7, and demonstrate that the compounds of this invention have anticancer activity against a wide variety of cancers.

TABLE 7
Potencies of SK inhibitors toward human tumor cell lines.
Sparsely plated cells were treated with an SK inhibitor for 48
hours, and cell viability was determined using sulforhodamine B
staining and compared to vehicle-(DMSO) treated cells. Values are
the mean ± sd for at least three separate experiments.

| | | IC₅₀ (μM) | IC₅₀ (μM) |
|---|---|---|---|
| Cell Line | Tissue | Compound 8 | Compound 73 |
| 1025LU | Melanoma | 30.1 ± 3.9 | 8.3 ± 2.4 |
| A-498 | Kidney | 38.3 ± 7.7 | 4.0 ± 1.9 |
| Caco-2 | colon | 2.6 ± 1.2 | 6.3 ± 5.2 |
| DU145 | prostate | 21.2 ± 1.3 | 6.5 ± 3.6 |
| Hep-G2 | liver | 81.5 ± 38.9 | 13.8 ± 8.7 |
| HT-29 | colon | 54.7 ± 0.1 | 29.9 ± 9.4 |
| MCF-7 | breast, ER+ | 25.2 ± 4.7 | 1.8 ± 0.7 |
| MDA-MB-231 | breast, ER− | 30.1 ± 3.9 | 26.6 ± 6.7 |
| Panc-1 | pancreas | 19.0 ± 2.5 | 6.7 ± 2.4 |
| SK-OV-3 | ovary | 23.7 ± 3.2 | 10.2 ± 0.3 |
| T24 | bladder | 25.2 ± 2.9 | 22.7 ± 4.8 |

### EXAMPLE 8

### In vivo Toxicity of SK Inhibitors of this Invention

For example, Compounds 8 and 73 were found to be soluble to at least 15 mg/ml (˜30-40 mM) in DMSO: PBS for intraperitoneal (IP) administration or PEG400 for oral dosing. Acute toxicity studies using IP dosing demonstrated no immediate or delayed toxicity in female Swiss-Webster mice treated with up to at least 50 mg/kg of Compounds 8 and 73. Repeated injections in the same mice every other day over 15 days showed similar lack of toxicity Each of the compounds could also be administered orally to mice at doses up to at least 100 mg/kg without noticeable toxicity.

### EXAMPLE 9

### Pharmacokinetics of a Representative SK Inhibitor of this Invention

Oral pharmacokinetic studies were performed on Compound 8. The compound was dissolved in PEG400 and administered to female Swiss-Webster mice at a dose of 100 mg/kg by oral gavage. Mice were anesthetized and blood was removed via cardiac puncture at 5 minutes, 30 minutes, 1, 2, and 8 hours. Concentrations of the test compounds were determined using liquid-liquid extraction, appropriate internal standards and reverse phase HPLC with UV detection. Control blood samples were run to identify compound-specific peaks. Pharmacokinetic parameters were calculated using the WINNONLIN analysis software package (Pharsight). Non-compartmental and compartmental models were tested, with the results shown in Table 8 derived from the best fit equations.

TABLE 8
Oral pharmacokinetic data for Compound 8.

| | Dose | AUC_{0 → ∞} | | tₘₐₓ | Cₘₐₓ | t_{1/2} |
|---|---|---|---|---|---|---|
| Compound | (mg/kg) | (μg*h/mL) | (μM*h) | (h) | (μM) | (h) |
| 8 | 100 | 475 | 1500 | 1.0 | 34.4 | 31.9 |

These studies demonstrate that a substantial amount of the SK inhibitor can be detected in the blood 1 h after oral dosing Compound 8 has excellent PK properties, with Area Under the Curve (AUC) and Cₘₐₓ (maximum concentration reached in the blood) values exceeding the IC₅₀ for recombinant SK catalytic activity, as well as for S1P formation in the intact cell model for at least 8 h. The high half-life suggests prolonged activity, which will diminish the need for frequent dosing regimens. These PK properties demonstrate that the compounds of this invention have excellent drug properties, specifically high oral availability with low toxicity.

### EXAMPLE 10

### Antitumor Activity of SK Inhibitors of this Invention

The antitumor activity of representative SK inhibitors were evaluated using a syngeneic tumor model that uses the mouse JC mammary adenocarcimona cell line growing subcutaneously in immunocompetent Balb/c mice (Lee et al., 2003, Oncol Res 14; 49), These cells express elevated levels of SK activity relative to non-transformed cells, as well as the multidrug resistance phenotype due to P-glycoprotein activity.

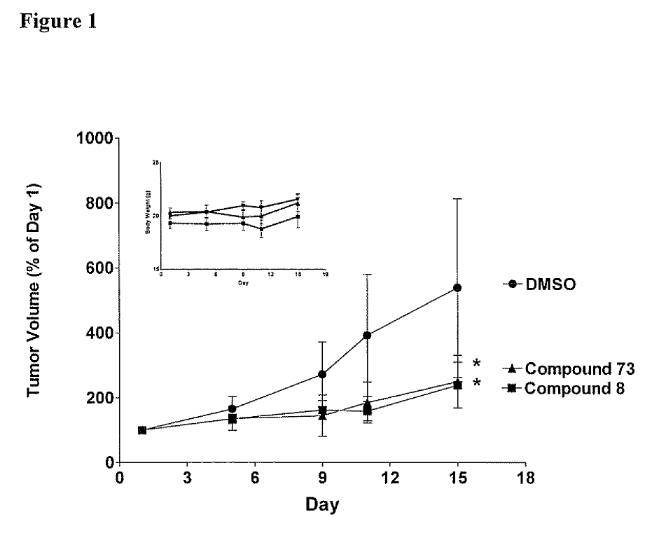

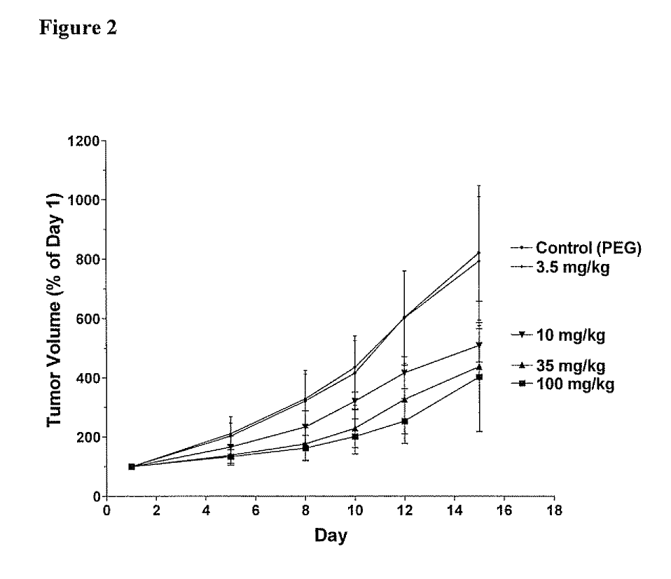

The data are shown in FIGS. 1 and 2. In FIG. 1, Balb/c mice, 6-8 weeks old, were injected subcutaneously with 1,000,000 JC cells suspended in phosphate-buffered saline. The SK inhibitors Compounds 8 and 73 were dissolved in 50% DMSO and administered by intraperitoneal injection to mice every-other day at a dose of 50 mg/kg. Body weights and tumor volumes were monitored daily. In FIG. 1, tumor growth is expressed as the tumor volume relative to day 1 for each animal.

As indicated in FIG. 1, tumor growth in animals treated with either SK inhibitor was significantly lower (>70% decreased at day 15) than tumor growth in control animals. Compounds 8 and 73 inhibited tumor growth relative to controls by 66 and 69%, respectively The insert of FIG. 1 indicates the body weight of the animals during this experiment. No significant difference in the body weights of animals in the three groups was observed, indicating the lack of overt toxicity from either SK inhibitor.

Dose-response studies with Compound 8 demonstrated that the compound has antitumor activity when orally administered at doses of 10 mg/kg or higher (FIG. 2). No toxicity to the mice was observed at any dose. The results are summarized in Table 9.

TABLE 9
In vivo antitumor activity of SK inhibitors. The indicated
compounds were tested in the JC tumor model using either
intraperitoneal (ip) or oral (po) administration. A compound is
indicated as being active if it suppressed tumor growth by at
least 60% relative to tumors in control animals.

| Compound | In vivo activity |
|---|---|
| 8 | active - ip and po |
| 73 | active - ip |

### EXAMPLE 11

### Inhibition of TNFα-Induced Prostaglandin Synthesis by SK Inhibitors

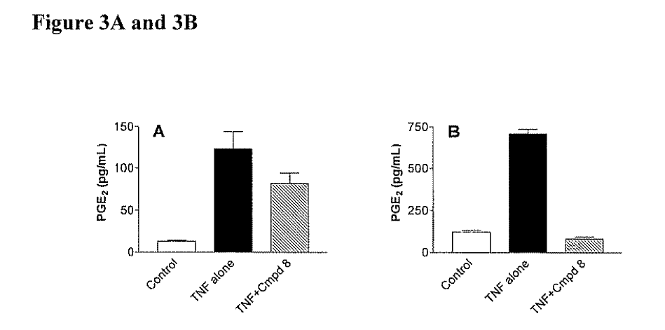

To determine the effects of the SK inhibitors on Cox-2 activity, an ELISA assay was used to measure PGE₂ production by IEC6 rat intestinal epithelial cells and human endothelial cells treated with TNFα. Exposure of either type of cell to TNFα resulted in marked increases in Cox-2 activity, measured as the production of PGE₂ (FIG. 3). This induction of Cox-2 activity by TNFα was strongly suppressed by Compound 8.

### EXAMPLE 12

### In vivo Effects of SK Inhibitors in an Acute Model of inflammatory Bowel Disease

Experiments were conducted with SK inhibitors using the dextran sulfate sodium (DSS) model of IBD. In these experiments, male C57BL/6 mice were provided with standard rodent diet and water ad libitum. After their acclimation, the animals were randomly divided into groups of 5 or 6 for DSS (40,000 MW from JCN Biomedicals, Inca, Aurora, Ohio)—and drug-treatment. The SK inhibitors were dissolved in PEG400, and given once daily by oral gavage in a volume of 0.1 mL per dose. Dipentum, an FDA-approved anti-colitis drug whose active ingredient, olsalazine, is converted to 5-aminosalicylic acid in vivo, was used as a positive control. The mice were given normal drinking water or 2% DSS and treated orally with an SK inhibitor or Dipentum at a dose of 50 mk/kg daily. The body weight of each animal was measured each day, and the Disease Activity Index (DAI) was scored for each animal on Days 4-6. On Day 6, the animals were sacrificed by cervical dislocation and the entire colon was removed and measured to the nearest 0.1 cm.

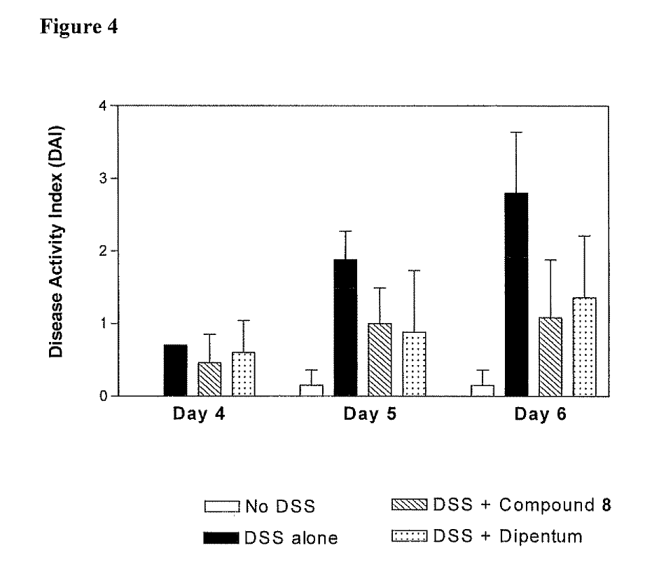

The drug-activity index (DAI) monitors weight loss, stool consistency and blood in the stool and is a measure of disease severity. Animals receiving normal drinking water and PEG as a solvent control had very low DAIs throughout the experiment (FIG. 4). Exposure of the mice to DSS in their drinking water markedly induced IBD symptoms, including weight loss and the production of loose, bloody stools. The intensity of the disease progressively increased from Day 4 to the time the mice were sacrificed on Day 6. Treatment of the animals receiving DSS with Compound 8 or Dipentum reduced the intensity of the IBD manifestations in the mice, most dramatically on Day 6. The SK inhibitors and Dipentum were essentially equivalent in their abilities to reduce the DAI of mice receiving DSS. It should be noted that this acute model produces rapid and dramatic symptoms of IBD, making it a very stringent assay for drug testing.

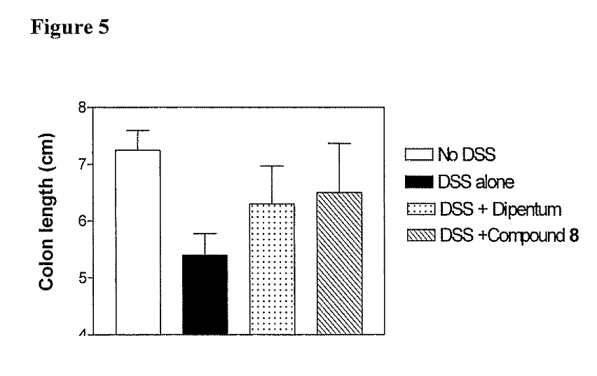

On Day 6, the animals were sacrificed by cervical dislocation and the entire colon was measured to assess shortening due to scarring and damage. Compared with the water control group, the colons of mice treated with DSS and PEG were significantly shortened (FIG. 5). DSS-treated mice that were also treated with Compound 8 or Dipentum had colons of intermediate length, indicating substantial protection by the drugs. Again, the response to the SK inhibitor was at least as good as that of mice treated with Dipentum.

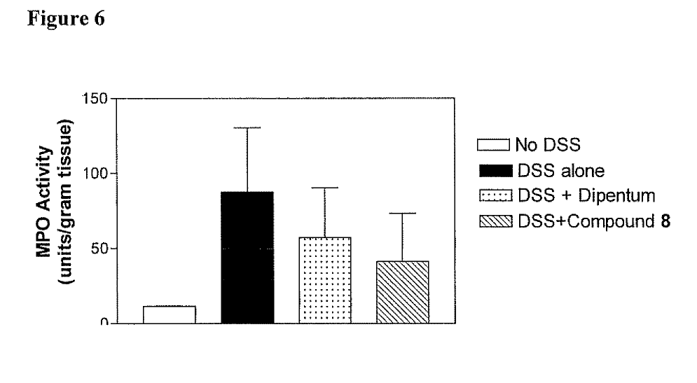

Myeloperoxidase (MPO) activity, which is reflective of neutrophil influx into the colon, is often used as measure of inflammation, and was assayed in the colons of the mice from the DSS-colitis studies. As indicated in FIG. 6, MPO activity was highly elevated in the DSS-alone animals compared to water controls. The increase in MPO activity was markedly attenuated in mice receiving daily doses of Compound 8 or Dipentum. This reduction in the activity of the neutrophil marker is consistent with the decreased occurrence of granulocytes observed in the H&E-stained colon sections. Therefore, the level of colonic MPO appears to be an excellent biomarker for the extent of tissue infiltration by inflammatory leukocytes.

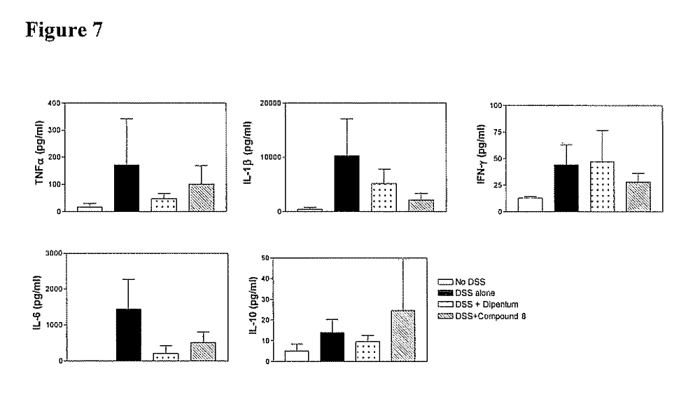

Several cytokines involved in inflammation were measured using the Luminex 100 System that allows the quantification of multiple cytokines and growth factors in a small sample volume. We examined the Th1 cytokine IFN-γ, the regulatory IL-10 cytokine, as well as the macrophage-derived pro-inflammatory cytokines, TNFα, IL-1β, IL-6 in colon samples from mice in the DSS model of colitis. FIG. 7 depicts the results of these assays, and indicates that DSS-treatment promoted the accumulation of all of the cytokines in the colon. Importantly, the elevations of all of the pro-inflammatory proteins, i.e. IFN-γ, IL-1β, IL-6 and TNFα, were attenuated in mice treated with either the SK inhibitor or Dipentum. Conversely, levels of the anti-inflammatory cytokine IL-10 were not suppressed by the SK inhibitor,

### EXAMPLE 13

### In vivo Effects of SK Inhibitors in a Chronic Model of Inflammatory Bowel Disease

A 35-day model of IBD was used to evaluate the effectiveness of the SK inhibitor in mice that experience multiple cycles of DSS-induced inflammation. This chronic model is similar to the acute model, except that the DSS concentration in the drinking water is lower and animals receive periodic exposure to DSS (DSS on days 1-7, water on Days 8-13, DSS on day 14-21, water on Days 22-27 and then DSS until the completion of the study on Day 35). In these experiments, treatment of the mice with an SK inhibitor or Dipentum began on Day 28 and continued daily until the completion of the study. The DAI index was monitored every other day until Day 28 and then daily until Day 35. Animals were sacrificed on Day 35, and changes in the colon length and cytokine profiles were measured.

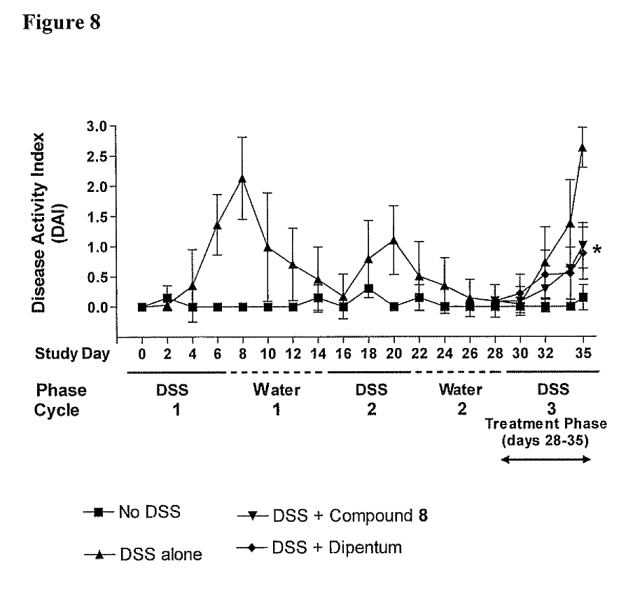

Cyclic exposure of mice to DSS in their drinking water caused reversible increases in the DAI (FIG. 8). Treatment of the mice with Compound 8 or Dipentum during the third exposure to DSS significantly suppressed the increase in DAI experienced by the control mice (P<0.001 on Day 35).

The colon lengths of DSS-treated mice were significantly shorter than the water-treated control animals (4.9±0.2 cm vs. 7.8±0.3 cm) reflecting inflammation-induced scarring. As in the acute model, the colons of animals treated with Compound 8 or Dipentum were of intermediate length (5.8±0.1 and 6.1±0.2 cm, respectively). This is a significant finding since the animals were untreated for the first and second DSS cycles. Therefore, suppression of inflammation-induced colon contraction can be reversed by effective anti-IBD drugs.

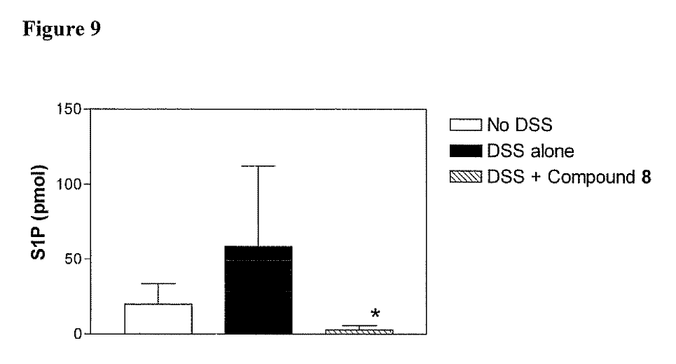

As a final measure of the effects of the SK inhibitors in this chronic model, S1P levels were assayed in the colons of the DSS-treated animals using an LC-MS/MS method. This technique allows us to examine correlations between biologic activity and changes in S1P levels in animals treated with the SK inhibitors. Samples of colons from animals from the DSS-colitis experiments were homogenized in cold PBS, spiked with internal standards (C₁₇ analogs of sphingosine and S1P) and processed by liquid-liquid extraction. Ratios of analyte to internal standard for each sphingolipid were determined. S1P levels were elevated in DSS alone treated mice as compared to water controls (FIG. 9). Treatment with Compound 8 (oral 50 mg/kg daily; 7 days prior to sacrifice) resulted in significant reductions of S1P levels (FIG. 9).

### EXAMPLE 14

### In vivo Effects of SK Inhibitors in the Collagen-Induced Arthritis Model in Mice

The anti-arthritis activities of the SK inhibitor Compound 8 were assessed in the Collagen-Induced Arthritis model Female DBA/1 mice were injected subcutaneously in the tail with chicken immunization-grade type II collagen emulsified in complete Freund's adjuvant at 2 mg/mL. Three weeks later, the mice received a collagen booster in incomplete Freund's adjuvant and were monitored daily thereafter for arthritic symptoms. Once mice reached a threshold paw thickness and clinical score, they were randomized into the following treatment groups: Compound 8 (50 mg/kg given orally each day for 6 days per week) or vehicle (Polyethyleneglycol 400 given under the same schedule). The severity of disease in each animal was quantified by measurement of the hind paw volume with digital calipers. Each paw was scored based upon perceived inflammatory activity, in which each paw receives a score of 0-3 as follows: 0 normal; 1=mild, but definite redness and swelling of the ankle or wrist, or apparent redness and swelling limited to individual digits, regardless of the number of affected digits; 2=moderate redness and swelling of the ankle and wrist and 3=severe redness and swelling of the entire paw including digits, with an overall score ranging from 0-12. Differences among treatment groups were tested using ANOVA.

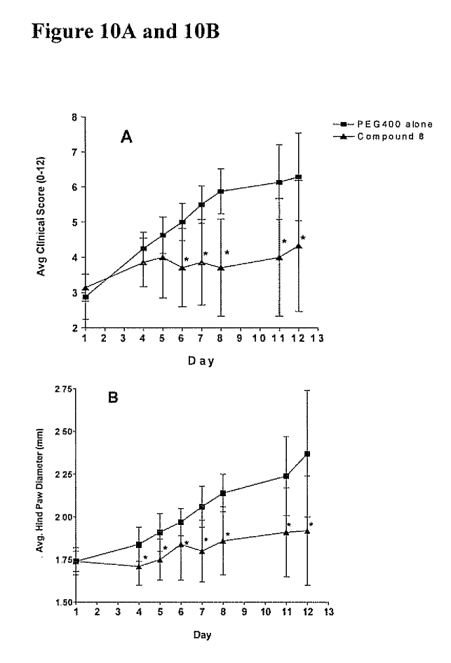

As indicated in FIG. 10, treatment with Compound 8 dramatically slowed the inflammation response, measured as either the Average Clinical Score (FIG. 10A) or the Average Hind Paw Diameter (FIG. 10B), with significant decreases beginning at Day 4 of treatment for both endpoints By the end of the experiment on Day 12, Compound 8 caused a 72% reduction in the increase in hind paw thickness, and a 65% reduction in clinical score compared with vehicle-treated mice. Since a 30% reduction in symptoms is considered demonstrative of anti-arthritic activity in this assay, the SK inhibitor surpasses the criteria for efficacy in this model.

On Day 12, the mice were euthanized and their hind limbs were removed, stripped of skin and muscle, formalin-fixed, decalcified and paraffin-embedded The limbs were then sectioned and stained with hematoxylin/eosin. Tibiotarsal joints were evaluated histologically for severity of inflammation and synovial hyperplasia. Collagen-Induced Arthritis resulted in a severe phenotype compared with non-induced mice, manifested as severe inflammation and synovial cell infiltration, as well as significant bone resorption. Mice that had been treated with Compound 8 had significantly reduced histologic damage, correlating with the paw thickness and clinical score data.

It should be understood that the foregoing disclosure emphasizes certain specific embodiments of the invention and that all modifications or alternatives equivalent thereto are within the spirit and scope of the invention as set forth in the appended claims. The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A compound of the formula I 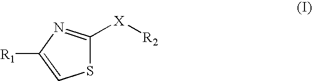
wherein:
X is —C(R₃,R₄)N(R₅)—, —C(O)NR₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;
R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above R₁ and R₂ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂; and
R₃ is H, alkyl, preferably lower alkyl, or oxo, provided that when R₃ and R₄ are on the same carbon, and R₃ is oxo, then R₄ is absent;
R₄ and R₅ are independently H or (C₁-C₆)alkyl,
or a pharmaceutically acceptable salt, hydrate or solvate thereof.

2. A compound of the formula II: 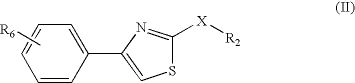
and pharmaceutically acceptable salts thereof, wherein:
X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CON₃R₄, —OC(O)NR₃R₄, —NR₃C(O)R₄, —CF₃, —OCF₃, —OH, C₁-C₆ alkoxy, hydroxyalkyl, —CN, —CO₂H, —SH, —S-alkyl, —SOR₃R₄, —SO₂R₃R₄, —NO₂, or NR₃R₄;
R₃ is H, alkyl, preferably lower alkyl, or oxo, provided that when R₃ and R₄ are on the same carbon, and R₃ is oxo, then R₄ is absent;
R₄ and R₅ are independently H or alkyl, preferably lower alkyl; and
R₆ is halogen, haloalkyl, alkoxy, haloalkoxy, hydroxyalkyl, alkanoyl, —COOH, —OH, —SH, —S-alkyl, —CN, —NO₂, or —NH₂.

3. A compound according to claim 2, that is:
1-[4-(4-Chloro-phenyl)-thiazol-2-yl]3-(4-chloro3-trifluoromethyl-phenyl)-urea,
Tetradecanoic acid [4-(4-chloro-phenyl)-thiazol-2-yl]-amide,
Hexadecanoic acid [4-(4-chloro-phenyl)-thiazol-2-yl]-amide,
Undec-10-enoic acid [4-(4-chloro-phenyl)-thiazol-2-yl]-amide,
N-[4-(4-Chloro-phenyl)-thiazol-2-yl]-3-(4-nitro-phenyl)-acrylamide,
Octadec-9-enoic acid [4-(4-chloro-phenyl)-thiazol-2-yl]-amide,
N-[4-(4-Chloro-phenyl)-thiazol-2-yl]-3-phenyl-acrylamide,
Butyric acid 4-{2-[4-(4-chloro-phenyl)-thiazol-2-ylcarbamoyl]-vinyl) -2-methoxy-phenyl ester,
N-[4-(3-Chloro-phenyl)-thiazol-2-yl]3-(4-hydroxy3-methoxy-phenyl)-acrylamide,
Acetic acid 4-{2-[4-(4-chloro-phenyl)-thiazol-2-ylcarbamoyl]-vinyl)-2-methoxy-phenyl ester,
Butyric acid 2-butyryloxy-5-{2-[4-(4-chloro-phenyl)-thiazol-2-ylcarbamoyl]-vinyl}-phenyl ester,
Acetic acid 4-{2-[4-(4-chloro-phenyl)-thiazol-2-ylcarbamoyl]-vinyl}-phenyl ester,
Butyric acid 2-{2-[4-(4-chloro-phenyl)-thiazol-]-2-ylcarbamoyl]-vinyl}-phenyl ester,
Butyric acid 3-{2-[4-(4-chloro-phenyl)-thiazol-2-ylcarbamoyl]-vinyl}-phenyl ester,
Butyric acid 4-{2-[4-(4-chloro-phenyl)-thiazol-2-ylcarbamoyl]-vinyl}-phenyl ester,
Butyric acid 4-{[4-(4-chloro-phenyl)-thiazol-2-ylcarbamoyl]-methyl}-2-methoxy-phenyl ester,
Butyric acid 2-butyryloxy-5-{[4-(4-chloro-phenyl)-thiazol-2-ylcarbamoyl}-methyl)-phenyl ester,
Butyric acid 5-{2-[4-(4-chloro-phenyl)-thiazol-2-ylcarbamoyl]-vinyl}-2-methoxy-phenyl ester,
Butyric acid 2-methoxy-4-[2-(4-p-tolyl-thiazol-2-ylcarbamoyl)-vinyl]-phenyl ester,
Butyric acid 4-{2-[4-(4-bromo-phenyl)-thiazol-2-ylcarbamoyl]-vinyl}-2-methoxy-phenyl ester,
3-Benzo[1,3]dioxol-5-yl-N-[4-(4-chloro-phenyl)-thiazol-2-yl]-acrylamide,
2-Benzo[1,3]dioxol-5-yl-N-[4-(4-chloro-phenyl)-thiazol-2-yl]-acetamide,
N-[4-(4-Chloro-phenyl)-thiazol-2-yl]3-(3,4-dimethoxy-phenyl)-propionamide,
Butyric acid 4-[4-(4-chloro-phenyl)-thiazol-2-ylcarbamoyl]-2-methoxy-phenyl ester,
Butyric acid 2-butyryloxy-4-[4-(4-chloro-phenyl)-thiazol-2-ylcarbamoyl]-phenyl ester,
Butyric acid 2-butyryloxy-4-{2-[4-(4-chloro-phenyl)-thiazol-2-ylcarbamoyl]-ethyl}-phenyl ester,
Butyric acid 2,6-bis-butyryloxy-4-[4-(4-chloro-phenyl)thiazol-2-ylcarbamoyl]-phenyl ester,
Butyric acid 4-{2-[4-(4-fluoro-phenyl)-thiazol-2-ylcarbamoyl]-vinyl}-2-methoxy-phenyl ester,
Butyric acid 4-{2-[4-(4-chloro-phenyl)-thiazol-2-ylcarbamoyl]-ethyl}-2-methoxy-phenyl ester,
Butyric acid 4-{[4-(4-chloro-phenyl)-thiazol-2-ylcarbamoyl]-methyl }-2-nitro-phenyl ester,
Butyric acid 2-amino-4-{[4-(4-chloro-phenyl)-thiazol-2-ylcarbamoyl]-methyl }-phenyl ester,
4-(4-Chloro-phenyl)-thiazole-2-carboxylic acid ethyl ester,
4-(4-Chloro-phenyl)-thiazole-2-carboxylic acid,
4-(4-Chloro-phenyl)thiazole-2-carboxylic acid (pyridin-4-ylmethyl)amide,
4-(4-Chloro-phenyl)-thiazole-2-carboxylic acid 4-dimethylamino-benzylamide,
4-(4-Chloro-phenyl)-thiazole-2-carboxylic acid 3,5-difluoro-benzylamide,
4-(4-Chloro-phenyl)-thiazole-2-carboxylic acid 4-chloro3-trifluoromethyl-benzylamide,
4-(4-Chloro-phenyl)-thiazole-2-carboxylic acid 2-chloro-4-fluoro-benzylamide,
4-(4-Chloro-phenyl)-thiazole-2-carboxylic acid 3-chloro-4-fluoro-benzylamide,
4-(4-Chloro-phenyl)-thiazole-2-carboxylic acid 3,4-difluoro-benzylamide,
4-(4-Chloro-phenyl)-thiazole-2-carboxylic acid [2-(3-bromo-4-methoxy-phenyl)-ethyl]-amide,
4-(4-Chloro-phenyl)-thiazole-2-carboxylic acid 3,4,5-trifluoro-benzylamide,
4-(4-Chloro-phenyl)-thiazole-2-carboxylic acid 3-trifluoromethoxy-benzylamide,
4-(4-Chloro-phenyl)-thiazole-2-carboxylic acid [2-(3-phenoxy-phenyl)-ethyl]-amide,
4-(4-Chloro-phenyl)-thiazole-2-carboxylic acid [2-(1-methyl-pyrrolidin-2-yl)-ethyl]-amide,
4-(4-Chloro-phenyl)-thiazole-2-carboxylic acid (4-methyl-piperazin-1-yl)-amide,
N-[4-(4-Chloro-phenyl)-thiazol-2-yl]-3-(2,4-difluoro-phenyl)-propionamide,
4-(4-Chloro-phenyl)-thiazole-2-carboxylic acid (2-ethylsulfanyl-ethyl)-amide,
4-(4-Chloro-phenyl)-thiazole-2-carboxylic acid 2-fluoro-4-trifluoromethyl-benzylamide,
4-(4-Chloro-phenyl)-thiazole-2-carboxylic acid (3,5-difluoro-phenyl)-amide,
4-(4-Chloro-phenyl)-thiazole-2-carboxylic acid 4-methylsulfanyl-benzylamide,
4-(4-Chloro-phenyl)-thiazole-2-carboxylic acid 4-trifluoromethoxy-benzylamide,
4-(4-Chloro-phenyl)-thiazole-2-carboxylic acid 4-fluoro3-trifluoromethyl-benzylamide,
4-(4-Chloro-phenyl)-thiazole-2-carboxylic acid 4-phenoxy-benzylamide,
4-(4-Chloro-phenyl)-thiazole-2-carboxylic acid (biphenyl-4-ylmethyl)-amide,
4-(4-Chloro-phenyl)-thiazole-2-carboxylic acid [1-(4-chloro-phenyl)-ethyl]-amide,
4-(4-Chloro-phenyl)-thiazole-2-carboxylic acid (3-tert-butylamino-propyl)-amide,
4-(4-Chloro-phenyl)-thiazole-2-carboxylic acid 4-trifluoromethyl-benzylamide,
4-(4-Chloro-phenyl)-thiazole-2-carboxylic acid (3-pyrrolidin-1-yl-propyl)-amide,
4-(4-Chloro-phenyl)-thiazole-2-carboxylic acid 3,5-bis-trifluoromethyl-benzylamide,
4-(4-Chloro-phenyl)-thiazole-2-carboxylic acid (2-pyridin-4-yl-ethyl)-amide,
4-(4-Chloro-phenyl)-thiazole-2-carboxylic acid (1H-tetrazol-5-yl)-amide,
4-(4-Chloro-phenyl)-thiazole-2-carboxylic acid 4-methanesulfonyl-benzylamide,
4-(4-Chloro-phenyl)-thiazole-2-carboxylic acid (2-benzo[1,3]dioxol-5-yl-ethyl)-amide,
N-[4-(4-Chloro-phenyl)-thiazol-2-yl]-3-fluoro-benzamide,
N-[4-(4-Chloro-phenyl)-thiazol-2-yl]-2-fluoro-4-trifluoromethyl-benzamide,
N-[4-(4-Chloro-phenyl)-thiazol-2-yl]-4-fluoro-benzamide,
2,4-Dichloro-N-[4-(4-chloro-phenyl)-thiazol-2-yl]-benzamide,
2-Chloro-N-[4-(4-chloro-phenyl)-thiazol-2-yl]-2-phenyl-acetamide,
N-[4-(4-Chloro-phenyl)-thiazol-2-yl]-2-(4-fluoro-phenyl)-acetamide,
[4-(4-Chloro-phenyl)thiazol-2-yl]-bis-(3-phenyl-propyl)-amine,
Dibenzyl-[4-(4-chloro-phenyl)-thiazol-2-yl]-amine,
Benzyl-[4-(4-chloro-phenyl)-thiazol-2-yl]-amine,
4-(4-Chloro-phenyl)-thiazole-2-carboxylic acid (2-pyridin-4-yl)-amide,
4-(4-Chloro-phenyl)-thiazole-2-carboxylic acid 3-fluoro-5-trifluoromethyl-benzylamide,
4-(4-Chloro-phenyl)-thiazole-2-carboxylic acid (2-morpholin-4-yl-ethyl)-amide,
[4-(4-Chloro-phenyl)-thiazol-2-yl]-(3,5-difluoro-phenoxymethyl)-amine,
[4-(4-Chloro-phenyl)-thiazol-2-yl]-(2,5-difluor o-phenoxymethyl)-amine,
[4-(4-Chloro-phenyl)-thiazol-2-yl]-(3,5-difluoro-benzyloxymethyl)-amine,
or pharmaceutically acceptable salts, hydrates or solvates thereof.

4. A compound of the formula III 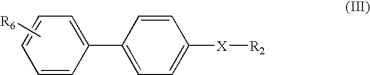
wherein:
x is —C(R₃,R₄)N(R₅)—, —C(O)N(R₄)—, —N(R₄)C(O)—, —C(R₄R₅)—, —N(R₄)—, —O—, —S—, —C(O)—, —S(O)₂—, —S(O)₂N(R₄)— or —N(R₄)S(O)₂—;
R₁ is halogen, haloalkyl, alkoxy, haloalkoxy, hydroxyalkyl, alkanoyl, —COOH, —OH, —SH, —S-alkyl, —CN, —NO₂, or —NH₂.
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CON₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and
R₃ is H, alkyl, preferably lower alkyl, or oxo, provided that when R₃ and R₄ are on the same carbon, and R₃ is oxo, then R₄ is absent;
R₄ and R₅ are independently H or alkyl, preferably lower alkyl,
or a pharmaceutically acceptable salt, hydrate or solvate thereof.

5. A compound according to claim 4, that is:
4′-Chloro-biphenyl-3-carboxylic acid [2-(1-methyl-pyrrolidin-2-yl)-ethyl]-amide,
4′-Chloro-biphenyl-3-carboxylic acid (pyridin-4-ylmethyl)-amide,
4′-Chloro-biphenyl-3-carboxylic acid (1-methyl-piperidin-4-yl)-amide,
4′-Chloro-biphenyl-3-carboxylic acid (4-hydroxy-phenyl)-amide,
4′-Chloro-biphenyl-3-carboxylic acid (2-pyridin-4-yl-ethyl)-amide,
(4′-Chloro-biphenyl-3-ylmethyl)-pyridin-4-ylmethyl-amine,
(4′-Chloro-biphenyl-3-ylmethyl)-[2-(1-methyl-pyrrolidin-2-yl)-ethyl]-amine,
or pharmaceutically acceptable salts, hydrates or solvates thereof

6. A compound of the formula IV 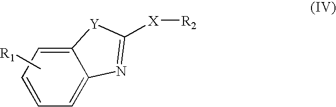
wherein:
X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;
Y is O or S;
R₁ is halogen, haloalkyl, alkoxy, haloalkoxy, hydroxyalkyl, alkanoyl, —COOH, —OH, —SH, —S-alkyl, —CN, —NO₂, or —NH₂;
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C-₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, -CO₂H, —SH, —S-alkyl, —SOR₄R₅, —SO₂R₄R₅, —NO₂, or NR₄R₅; and
R₃ is H, alkyl, preferably lower alkyl, or oxo, provided that when R₃ and R₄ are on the same carbon, and R₃ is oxo, then R₄ is absent;
R₄ and R₅ are independently H or alkyl, preferably lower alkyl,
or a pharmaceutically acceptable salt, hydrate or solvate thereof.

7. A compound according to claim 6, that is:
N-(5-Chloro-benzooxazol-2-yl)-2-nitro-benzamide,
N-(5-Chloro-benzooxazol-2-yl)3-phenyl-acrylamide,
N-(5-Chloro-benzooxazol-2-yl)3-(4-nitro-phenyl)-acrylamide,
Undec-10-enoic acid (5-chloro-benzooxazol-2-yl)-amide,
Tetradecanoic acid (5-chloro-benzooxazol-2-yl)-amide,
Hexadecanoic acid (5-chloro-benzooxazol-2-yl)-amide,
1-(5-Chloro-benzooxazol-2-yl)3-(4-chloro3-trifluoromethyl-phenyl)-urea,
1-Benzothiazol-2-yl-3-(4-chloro3-trifluoromethyl-phenyl)-urea,
Butyric acid 4-[(6-chloro-benzothiazol-2-ylcarbamoyl)-methyl]-2-methoxy-phenyl ester,
N-(5-Chloro-benzothiazol-2-yl)-2-hydroxy-benzamide,
N-(5-Chloro-benzooxazol-2-yl)3-fluoro-benzamide,
or pharmaceutically acceptable salts, hydrates or solvates thereof.

8. A pharmaceutical composition comprising a compound according to claims 1, 2, 3, 4, 5, 6, or 7, or a pharmaceutically acceptable salt, hydrate or solvate thereof, in combination with a pharmaceutically acceptable carrier, medium, or auxiliary agent

9. A method of inhibiting of sphingosine kinase in a patient in need of such inhibition, the method comprising administering to a the patient in need of such inhibition a compound or salt according to claims 1, 2, 3, 4, 5, 6, or 7, with or without a combination of a pharmaceutically acceptable carrier, medium, or auxiliary agent,

10. A method of treating a disorder in a patient, said disorder having abnormal activation of sphingosine kinase, the method comprising administration to the patient of a therapeutically effective amount of a compound or salt according to claim 1, or a composition comprising a compound or salt according to claims 1, 2, 3, 4, 5, 6, or 7, with or without a combination with a pharmaceutically acceptable carrier, medium, or auxiliary agent.

11. A method for treating a disease that is a hyperproliferative disease, an inflammatory disease, or an angiogenic disease comprising administration to a patient in need of such treatment of a therapeutically effective amount of a sphingosine kinase inhibiton compound or salt according to claims 1, 2, 3, 4, 5, 6, or 7, with or without a combination comprising a compound or salt according to claim 1 in combination with a pharmaceutically acceptable carrier, medium, or auxiliary agent.

12. The method of claim 11 wherein said hyperproliferative disease is cancer, atherosclerosis, restenosis, mesangial cell proliferative disorders, or psoriasis

13. The method of claim 12 wherein said cancer is head and neck cancers, lung cancers, gastrointestinal tract cancers, breast cancers, gynecologic cancers, testicular cancers, urinary tract cancers, neurological cancers, endocrine cancers, skin cancers, sarcomas, mediastinal cancers, retroperitoneal cancers, cardiovascular cancers, mastocytosis, carcinosarcomas, cylindroma, dental cancers, esthesioneuroblastoma, urachal cancer, Merkel cell carcinoma, paragangliomas, Hodgkin lymphoma, non-Hodgkin lymphoma, chronic leukemias, acute leukemias, myeloproliferative cancers, plasma cell dyscrasias, or myelodysplastic syndromes

14. The method of claim 12 wherein said mesangial cell proliferative disorders is glomerulonephritis, diabetic nephropathy, malignant nephrosclerosis, thrombotic microangiopathy syndromes, transplant rejection, or glomerulopathies

15. The method of claim 11 wherein said inflammatory disease is inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis or T cell-mediated diseases of immunity.

16. The method of claim 15 wherein said inflammatory bowel disease is ulcerative colitis, Crohn's Disease or indeterminate colitis.

17. The method of claim 15 wherein said T cell-mediated diseases of immunity is allergic encephalomyelitis, allergic neuritis, transplant allograft rejection, graft versus host disease, myocarditis, thyroiditis, nephritis, systemic lupus erythematosus, or insulin-dependent diabetes mellitus.

18. The method of claim 15 wherein said arthritis is rheumatoid arthritis, osteoarthritis, Caplan's Syndrome, Felty's Syndrome, Sjogren's Syndrome, ankylosing spondylitis, Still's Disease, Chondrocalcinosis, gout, rheumatic fever, Reiter's Disease or Wissler's Syndrome.

19. The method of claim 15 wherein said inflammatory kidney disease is glomerulonephritis, glomerular injury, nephrotic syndrome, interstitial nephritis, lupus nephritis, Goodpasture's disease, Wegener'S granulomatosis, renal vasculitis, IgA nephropathy or idiopathic glomerular disease.

20. The method of claim 15 wherein said skin inflammation is psoriasis, atopic dermatitis, contact sensitivity or acne.

21. The method of claim 19 wherein said angiogenic disease is diabetic retinopathy, arthritis, cancer, psoriasis, Kaposi's sarcoma, hemangiomas, myocardial angiogenesis, atherosclerotic plaque neovascularization, and ocular angiogenic diseases such as choroidal neovascularization, retinopathy of prematurity (retrolental fibroplasias), macular degeneration, corneal graft rejection, rubeosis, neuroscular glacoma or Oster Webber syndrome.
